(19) United States
(12) Patent Application Publication
(10) Pub. No.: US 2025/0388866 A1
(43) Pub. Date: Dec. 25, 2025

(54) SYSTEMS AND METHODS FOR IN VITRO AND IN VIVO LIVER ORGANOID GROWTH AND DIFFERENTIATION

(71) Applicant: Khufu Therapeutics, Inc., San Francisco, CA (US)

(72) Inventor: Natesh Parashurama, Getzville, NY (US)

(73) Assignee: Khufu Therapeutics, Inc., San Francisco, CA (US)

(21) Appl. No.: 18/995,271

(22) PCT Filed: Jul. 14, 2023
(86) PCT No.: PCT/US2023/070292

§ 371 Date: Jan. 16, 2025

Related U.S. Application Data
(60) Provisional application No. 63/390,625, filed on Jul. 19, 2022.

(51) Int. Cl. C12N 5/071

(52) U.S. Cl. CPC C12N 5/0671; C12N 2500/02; C12N 2500/38; C12N 2500/99; C12N 2501/105; C12N 2501/11; C12N 2501/115; C12N 2501/12; C12N 2501/155; C12N 2501/165; C12N 2501/91; C12N 2502/1323; C12N 2506/45; C12N 2513/00; C12N 2533/54; C12N 2533/90

(57) ABSTRACT

The present disclosure generally relates to systems and methods for growing liver cells, e.g., in vitro or in vivo. For instance, some aspects are generally directed to systems and methods of growing stem cells, such as pluripotent stem cells, to form liver cells, liver tissues, liver organoids, or the like. In some cases, the cells may be grown under hypoxic conditions. Without wishing to be bound by any theory, it is believed that such conditions may allow the stem cells to grow without necessarily differentiating, thereby producing large volumes of tissues that can subsequently mature to form liver structures. Other aspects are generally directed to cells, tissues, organoids, or other architectures formed from such methods, treatments of subjects involving such methods, kits using such methods, and the like.

### RELATED APPLICATIONS

This application claims the benefit of U.S. Provisional Patent Application Ser. No. 63/390,625, filed Jul. 19, 2022, entitled “Systems and Methods for in Vitro and in Vivo Liver Organoid Growth and Differentiation,” incorporated herein by reference in its entirety.

### FIELD

The present disclosure generally relates to systems and methods for growing liver cells, e.g., in vitro or in vivo.

### BACKGROUND

Chronic liver disease is escalating globally and currently affects more than 800 million people worldwide. The current accepted treatment is orthotopic liver transplantation, which bears numerous limitations, and liver regenerative medicine offers a wide array of promising several alternate solutions, of which liver organogenesis (LO) has great potential. The aim of LO is to recreate liver-like, functional tissues from adult stem cells or human pluripotent stem cells (hPSC), which would supersede many limitations of existing solutions. These functional tissues can then be used to isolate patient-specific hepatocytes (HEPs), or be used en bloc, for various in vitro applications as well as therapeutic transplantation. How to fully unravel the potential of LO remains an unanswered question in the field.

### SUMMARY

The present disclosure generally relates to systems and methods for growing liver cells, e.g., in vitro or in vivo. The subject matter of the present disclosure involves, in some cases, interrelated products, alternative solutions to a particular problem, and/or a plurality of different uses of one or more systems and/or articles.

One set of embodiments is generally directed to a method comprising growing endodermic cells in an environment comprising a hepatic medium having less than 60 mmHg O₂ partial pressure to produce heptaoblasts, wherein the endodermic cells are exposed to the environment for at least 8 days.

Another set of embodiments is generally directed to a method comprising growing pluripotent stem cells in an environment comprising a hepatic medium having less than 60 mmHg O₂ partial pressure, wherein the cells are exposed to the environment for at least 4 days; exposing the pluripotent stem cells to fibroblasts; and exposing the pluripotent stem cells and fibroblasts to a basement membrane matrix.

Yet another set of embodiments is generally directed to a method comprising growing pluripotent stem cells and fibroblasts in an environment comprising a hepatic medium to form a structure; and implanting the structure in the skin of a subject.

In another aspect, the present disclosure encompasses methods of making one or more of the embodiments described herein, for example, liver organoids. In still another aspect, the present disclosure encompasses methods of using one or more of the embodiments described herein, for example, liver organoids.

Certain embodiments are directed to unique liver protocols with relatively high maturation (liver score) without any maturation factors. In some cases, no transcription factors are added. In some embodiments, by incorporating migration, this is the equivalent of forced reprogramming of the cells.

Some embodiments are generally directed to imposing a growth to differentiation switch can increase transcriptional maturation.

Some embodiments are generally directed to a migration/growth to differentiation switch. This can be determined, for example, by changes in transcription factor expression, signaling, and transcriptional maturity. This may be performed in vitro or in vivo.

In some embodiments, the growth to differentiation switch can be used for certain in vitro and in vivo applications, for example, restoring function upon transplantation.

In some embodiments, migration may be present in lung, pancreas, thyroid, intestine, prostate, bladder, and mammary gland tissues. These may include similar mechanisms.

In some embodiments, transcriptional maturation may be used for determining differentiation, e.g., prior to studying functional maturation. Certain embodiments are directed to cells having a growth to differentiation switch. Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure.

### DETAILED DESCRIPTION

The present disclosure generally relates to systems and methods for growing liver cells, e.g., in vitro or in vivo. For instance, some aspects are generally directed to systems and methods of growing stem cells, such as pluripotent stem cells, to form liver cells, liver tissues, liver organoids, or the like. In some cases, the cells may be grown under hypoxic conditions. Without wishing to be bound by any theory, it is believed that such conditions may allow the stem cells to grow without necessarily differentiating, thereby producing larger volumes of tissues that can subsequently mature to form liver structures. Other aspects are generally directed to cells, tissues, organoids, or other architectures formed from such methods, treatments of subjects involving such methods, kits using such methods, and the like.

In one aspect, certain originating cells are grown in media that may induce the stem cells to grow and form liver-producing cells such hepatoblasts. Examples include stem cells such as pluripotent stem cells, and/or endodermic cells. The endodermic cells may be grown from stem cells in certain embodiments.

The originating cells may be grown under low-oxygen or hypoxic conditions in certain embodiments. In some cases, the cells may be grown without exposing the cells to growth factors, steroids, or other components which may cause the pluripotent stem cells to differentiate too rapidly. In some cases, the cells may be grown under such condition without changing the media type, e.g., by exposing the cells to different growth factors, steroids, or other components, etc. For instance, the media may be unchanged during such exposure, or there may be one or more changes in media, where it is relaced with media of the same type or same starting composition.

In one set of embodiments, the cells may be grown for at least 4 days, at least 6 days, at least 8 days, at least 10 days, at least 12 days, or at least 14 days without exposing the cells to such conditions. In some cases, the cells may be grown under such conditions from day 4 to at least day 14. Without wishing to be bound by any theory, it is believed that by growing under such conditions, larger amounts of cells or tissues may be produced, e.g., forming an organoid, without allowing the cells to differentiate too rapidly. In contrast, in many prior art techniques, a variety of growth or differentiating factors are added to the stem cells to induce them to differentiate quickly, e.g., to form liver cells.

The cells may be grown under such hypoxic conditions under in vitro conditions, in vivo conditions, or a combination of in vitro and in vivo conditions in certain cases. As one non-limiting example, originating cells may be grown in vitro under a hypoxic environment in a reactor for 2, 4, 6, 8, 10, 12, 14, or more days, and in some cases, without changing the media type. In some embodiments, the originating cells may be caused to form an organoid (e.g., a liver organoid), or other structure. As another non-limiting example, originating cells (for example, stem cells such as pluripotent stem cells) may be grown in vitro under a hypoxic environment for 4 or more days, exposed to fibroblasts and/or a basement membrane matrix (for instance, Matrigel), and implanted into the skin or other location within the body that exhibits relatively low oxygen partial pressures and grown in vivo within the skin, e.g., to form a structure, such as an organoid. In some cases, such structures may exhibit liver architecture, and in certain embodiments, such structures may be implanted into a subject, e.g., into the hepatic region of a subject.

The above discussion is a non-limiting example of certain embodiments generally directed to systems and methods for growing liver cells under hypoxic conditions, e.g., in vitro or in vivo. However, other embodiments are also possible. Accordingly, more generally, various aspects of the invention are directed to various systems and methods for systems and methods for growing liver cells.

For instance, certain aspects of the present disclosure are generally directed to systems and methods for inducing certain originating cells to form hepatoblasts, and in some cases, liver tissues, liver organoids, or other structures. Examples of originating cells include, but are not limited to, stem cells such as pluripotent stem cells, or endodermic cells. In some cases, some structures may be produced that exhibit liver architectures, e.g., exhibiting a plurality of multi-sided units known as the hepatic lobules drained by various veins.

In some cases, the mass of the resulting organoid or other structure, e.g., produced as discussed herein, may be substantially greater than the mass of the initial stem cells. For instance, the organoid or other structure may exhibit a mass of at least 100×, at least 300×, at least 500×, at least 1000×, at least 3000×, at least 5000×, at least 10,000×, at least 30,000×, etc. the mass of the initial stem cells. Without wishing to be bound by any theory, it is believed that in many prior art techniques involving stem cells, the differentiation of the stem cells occurs too quickly, e.g., due to the growth conditions, resulting in tissues or structures that are less massive.

The originating cells may be human cells, or non-human stem cells, e.g., arising from a non-human mammal, such as a monkey, cow, sheep, goat, horse, rabbit, pig, mouse, rat, dog, or cat, etc. The cells may also be naturally-occurring and/or genetically engineered in certain cases.

The originating cells may include, in certain embodiments, stem cells such as pluripotent stem cells. The pluripotent stem cells may be induced in some cases (i.e., induced pluripotent stem cells or iPSCs). In some embodiments, some or all of the cells may be partially differentiated. In addition, in some cases, the stem cells may comprise embryonic stem cells, or other types of stem cells.

In one set of embodiments, the stem cells may be grown to produce endodermic cells. For instance, in some cases, the stem cells may be exposed to an initial media that is able to induce the pluripotent stem cells to produce the endodermic cells. In some cases, the stem cells may be grown in a hypoxic environment, e.g., as discussed herein. Such media may be commercially obtained in certain instances. In certain cases, such stem cells may be grown for at least 1, 2, 3, 4, 5, or more days, e.g., to produce endodermic cells.

In one aspect, originating cells such as stem cells and/or endodermic cells (e.g., produced from the stem cells) may be grown in a hypoxic environment. Typically, a hypoxic environment has an oxygen concentration or partial pressure that is below phycological (resting) conditions. For instance, in an hypoxic environment, the partial pressure of oxygen may be less 160 mmHg, less than 140 mmHg, less than 120 mmHg, less than 100 mmHg, less than 80 mmHg, less than 70 mmHg, less than 60 mmHg, less than 50 mmHg, less than 40 mmHg, etc.

A variety of methods may be used to control the environment to render it hypoxic. For instance, in vivo, cells such as originating cells may be grown in an environment that is physiologically low in oxygen, for example, in a subcutaneous portion of the skin, or in a venous region. Thus, in one set of embodiments, such cells (or structures produced by such cells) may be implanted and grown in vivo within such regions within a subject.

As another non-limiting example, cells may be grow in vitro in an environment with lower or hypoxic concentrations of oxygen, e.g., for extended periods of time such as discussed herein. For instance, the cells may be grown or cultured in environments having gaseous concentrations of less than 10%, less than 9%, less than 8%, less than 7%, less than 6%, less than 5%, less than 4%, less than 3%, etc. of oxygen (percentages by volume).

In some aspects, originating cells may be grown in a medium, such as a hepatic medium. The hepatic medium may allow originating cells such as stem cells or endodermic cells to differentiate into hepatic and/or mesenchymal cells. For instance, the hepatic medium may comprise certain growth factors such as insulin growth factor (IGF), epidermal growth factor (EGF), fibroblast growth factor 2 (FGF2), vascular endothelial growth factor (VEGF), or the like. In some cases, the hepatic medium may also comprise heparin. In addition, in some cases, the hepatic medium may be free of serum, and/or comprise a serum replacement, such as KOSR (knockout serum replacement). In certain embodiments, the hepatic medium is also free of steroids.

In one aspect, the cells may be grown under such conditions (e.g., in an hypoxic environment, and/or with a hepatic medium, etc.) for relatively long periods of time. For example, in one set of embodiments, the cells may be grown under such conditions for 1, 2, 3, 4, 5, 6, 7, 8, 9, 10, 11, 12, 13, 14, or more days.

In some embodiments, the cells may be grown in vitro under such conditions without changing the media type. For instance, the media may be left unchanged, or the media may be changed (e.g., replaced with fresh media), but the media is of the same type or has substantially the same starting components and/or composition as before. In certain embodiments, such cells may grow and at least partially differentiate to form liver tissues, organoids, or other structures e.g., in an in vitro environment. Such organoids or other structures are discussed are discussed in more detail herein.

In some embodiments, the originating cells may be grown in a cell culture system, for example, using bioreactors, flasks, petri dishes, microwell plates (for example, 96- or 384-well plates), or other cell culture systems. Many cell culture systems will be known to those of ordinary skill in the art.

In addition, it should be understood that in some aspects, originating cells may be grown in a hypoxic environment in an in vivo setting (e.g., in a subject), rather than an in vitro setting. For instance, in one set of embodiments, originating cells such as stem cells or endodermic cells may be grown in the skin or other location within the body that exhibits relatively low oxygen partial pressures. For instance, the cells (or structures containing such cells) may be implanted and grown in a subcutaneous portion of the skin, or in a venous region.

The cells may be grown in such environments for any suitable number of days, e.g., for 1, 2, 3, 4, 5, 6, 7, 8, 9, 10, 11, 12, 13, 14, or more days. In addition, in some embodiments, the cells may be grown in vitro (e.g., such as is described herein), prior to implantation into a subject. For instance, the cells may be grown in vitro for 1, 2, 3, 4, 5, 6, 7, 8, or more days prior to implantation and growth in vivo.

In certain aspects, originating cells such as stem cells or endodermic cells may be exposed to fibroblasts, e.g., for growth in vivo and/or in vitro. The cells may be exposed to fibroblasts at the start of culture (e.g., day 0), or the fibroblasts may be introduced afterwards (e.g., after 1, 2, 3, 4, 5, 6, 7, 8, or more days). In certain instances, the originating cells and the fibroblasts may come from the same or different species. In some cases, the originating cells and the fibroblasts may come from the same subject.

Any of a variety of different fibroblast types may be used in various embodiments. In some cases, one or more than one type of fibroblast may be used, e.g., from the same or different species. One example of a fibroblast is a foreskin fibroblast (e.g., human foreskin fibroblasts). Additional non-limiting examples include skin (dermal) fibroblasts, pericytes, cardiac fibroblasts, muscular fibroblasts, etc.

In some embodiments, the fibroblasts may be added to the originating cells (e.g., stem cells and/or endodermic cells) at a ratio of at least 2:1, at least 3:1, or at least 4:1 of originating cells: fibroblasts. In addition, in some embodiments, the fibroblasts may be added to the stem cells at a ratio of no more than 6:1, no more than 5:1, or no more than 4:1 of originating cells: fibroblasts. Combinations of any of these ranges are also possible in certain cases, e.g., the fibroblasts may be present at a ratio of between 2:1 and 6:1. In one embodiment, the ratio of originating cells: fibroblasts is about 4:1.

In addition, in various aspects, the originating cells (and fibroblasts, if present) may be exposed to a basement membrane matrix. More than such basement membrane matrix material may be present in certain embodiments. Non-limiting examples include Matrigel, collagen, laminin, fibronectin, etc. In some cases, the Matrigel may be growth-factor free Matrigel. Matrigel is generally a solubilized basement membrane matrix secreted by Engelbreth-Holm-Swarm (EHS) mouse sarcoma cells, and resembles the laminin/collagen IV-rich basement membrane extracellular environment found in many tissues.

In some cases, the basement membrane matrix may be present at a concentration of at least 1 microliters of basement membrane matrix material per 10⁶ cells, and in some embodiments, at least 2 microliters, at least 3, microliters, at least 5 microliters, at least 10 microliters, at least 20 microliters, at least 30 microliters, at least 50 microliters, at least 100 microliters, at least 200 microliters, at least 300 microliters, at least 500 microliters, etc. of basement membrane matrix material per 10⁶ cells. In some embodiments, the basement membrane matrix may be present at a concentration of no more than 500 microliters of basement membrane matrix material per 10⁶ cells, and in certain instances, no more than 300 microliters, no more than 200 microliters, no more than 100 microliters, no more than 50 microliters, no more than 30 microliters, no more than 20 microliters, no more than 10 microliters, no more than 5 microliters, no more than 3 microliters, no more than 2 microliters, etc. of basement membrane matrix material per 106 cells. In certain cases, combinations of any of these ranges are possible, e.g., the concentration may be between 30 and 100 microliters of basement membrane matrix material per 10⁶ cells, between 50 and 200 microliters/10⁶ cells, between 10 and 100 microliters/10⁶ cells, etc.

The liver organoids or other structures, in certain aspects, may exhibit a three-dimensional structure or architecture that resembles liver. For instance, after formation and/or differentiation the organoid or other structure may exhibit a plurality of hepatic lobules drained by various vein-like structures.

In certain aspects, organoids or other structures may be caused to mature by exposing the originating cells to growth or other factors that induce differentiation, e.g., to cause the cells to from mature liver cells or hepatoblasts. For instance, cells, organoids, or other structures may be exposed to one or more of BMP4, higher FGF2, HGF, dexamethasone, oncostatin, or vitamin D. In some cases, the cells may be caused to mature after the originating cells have been grown for a period of time, e.g., for 1, 2, 3, 4, 5, 6, 7, 8, 9, 10, 11, 12, 13, 14, or more days.

In some aspects, the organoids or other structures may be induced to form liver architecture and/or vascularization. For instance, the organoid or other structure may exhibit venous or blood vessels after vascularization. In some cases, this may occur during and/or after growing the originating cells, e.g., for 1, 2, 3, 4, 5, 6, 7, 8, 9, 10, 11, 12, 13, 14, or more days. For instance, in some cases, an organoid or other structure may be caused to vascularize to allow hepatic lobules within the organoid or other structure to drain, e.g., into blood vessels. This process may occur in vitro and/or in vivo in some cases. A variety of techniques may be used to cause vascularization to occur. For instance, in one embodiment, originating cells and/or organoids or other structures may be exposed to one or more stimulators such as VEGF, FGF, VEGFR, NRP-1, Ang1, Ang2, PDGF, PDGFR, TGF-beta, endoglin, CCL2 histamine, integrins (e.g., alpha-v-beta-3, alpha-v-beta-5, alpha-5-beta-1), VE-cadherin, CD31, ephrin, plasminogen activators, eNOS, COX-2, AC133, ID1, ID3, class 3 semaphorins, Nogo-A, etc.

In addition, in one aspect, liver organoids (or other structures) grown in vitro and/or in vivo, including any of those described herein, may be implanted into a subject. An entire organoid or structure, or only a part of an organoid or other structure, may be transplanted.

The subject may be a human subject, or a non-human subject such as a monkey, cow, sheep, goat, horse, rabbit, pig, mouse, rat, dog, or cat, etc. The organoid or other structure may be implanted into the hepatic region of the subject (e.g., within or near the liver), or in some cases, the organoid or other structure may be implanted into other locations within the subject. If the organoid or other structure is grown in vivo, it may be implanted into the same subject or a different subject than the one in which it was grown in vivo.

The subject may be one that has a liver disease, in some embodiments. Non-limiting examples include non-alcoholic fatty liver disease (NAFLD), cirrhosis (e.g., NASH, alcoholic, viral, cholestatic, etc.). liver cancer, pediatric liver disease (acute, chronic, cancer), or the like. In some cases, an organoid or other structure may be implanted to a subject having a need for a liver transplant, e.g., due to a failed or failing liver. The organoid or other structure may be grown (e.g., as discussed herein) from the subject's own cells (e.g., stem cells and/or other cells taken from the subject), or from a different subject (e.g., one of the same or different species as the subject). In some cases, the organoid or other structure is grown from embryonic stem cells.

In some embodiments, liver organoids or other structures transplanted into the liver of a subject (e.g., a diseased liver) may expand and replace some or all of the diseased liver, which may help to at least partially restore liver function within the subject. In some cases, the liver organoid or other structure may be delivered using minimally invasive techniques, e.g., along the portal vein.

U.S. Provisional Patent Application Ser. No. 63/390,625, filed Jul. 19, 2022, entitled “Systems and Methods for in Vitro and in Vivo Liver Organoid Growth and Differentiation,” is incorporated herein by reference in its entirety.

The following examples are intended to illustrate certain embodiments of the present disclosure, but do not exemplify the full scope of the disclosure.

### EXAMPLE 1

This example demonstrates that E9.0 LD-MESC functions as a signaling center which initiates and executes these complex steps of liver organogenesis. This is based on the observation that at E9.0, the liver diverticulum (LD) forms and interacts with surrounding mesoderm (MES)-bearing tissue complex (MESC). There is a need to elucidate the factors that drive the E9.0 LD to give rise to 9.5 migrating hepatoblasts (MH).

To more deeply understand liver organogenesis, in this example, in-depth bioinformatic analysis of the scRNA-seq data for E8.5-E10.5 mouse hepatic cells was performed. As discussed below, it was found that:

To elicit transcriptional changes, 3 approaches including REACTOME, DAVID pathway analysis, and ENRICHR (gene list) were used. These include a new ranking technique to rank pathways that change with time, and a new method for averaging genes across a pathway.

Over 30 soluble factors and soluble factor mediators were found. These were used to define a culture medium for human LD experiments. Soluble signaling pathways were globally upregulated, including IGF-1, EGF, VEGF, FGF, WNT, TGF-β (TGF-beta), Hedgehog, Notch pathways, Neurotrophin, and Thyroid. Further, immune signaling (NF-kB and NFAT) was upregulated, as was overall gene transcription. Many of these overlap with regeneration.

A broad cell stress response within the E9.5 MH population was identified, including included PI3-Akt (mechanical stress), Hippo (mechanical stress), HIF1α (H1F1 alpha) (oxygen stress), mTOR (nutrient stress), AMPK (energy stress), DNA damage response (replicative stress) and FoxO (oxidative stress).

Migrating hepatoblasts were shown to indeed be migrating, based on upregulation of pathways associated with collective cell migration. It was found that migration is associated with pathways connected to chromatin modification.

Increased immune signaling, including series of pro and inflammatory cytokines, was observed.

Pie chart data demonstrated the extreme changes in transition from E9.5 to E10.5 hepatoblast population. Compared to E9.5, at E10.5 there was massive downregulation of both signal transduction and transcription, and extensive upregulation of both metabolism and protein metabolism, indicating the E9.5 population is indeed unique.

The method of averaging across all genes, for all cells, in each cluster, for a single pathway, was used to demonstrate certain findings. Coordinated upregulation of the following were observed: Hippo, TGF-B, WNT, Pluripotency, Branching morphogenesis, Migration, Hypoxia, and Liver genes. In addition, coordinated downregulation of the following were observed: Oxidative phosphorylation, and TCA cycle. Interestingly, it was found that coordinated FOXA2 expression was present in the migrating population. A relative downregulation of the master transcription factor, FOXA2, was also found.

Hepatomesenchyme and hepatoblast analysis. Both the transcriptome and niche of the E10.5 hepatoblast and the E10.5 hepatomesenchyme were defined.

These data provide a transcriptomic signature and the niche of the E9.5 migrating hepatoblast population, which may be central to organogenesis, but has been ignored until this time, in addition the analysis of the E10.5 hepatoblasts and hepatomesenchyme.

Next, a human hLD-MESC in vivo transplant model was demonstrated. After 4 weeks of subcutaneous transplantation, the hLD-MESC demonstrates extensive growth (2 orders of magnitude more than expected), liver differentiation (highest level of Albumin transcription measured in our lab), morphogenesis, and a lack of blood vessels. Findings were unique compared to other liver organogenesis studies.

These principles and the knowledge gained by both the bioinformatics analysis and the in vivo model were applied to develop an in vitro model of the LD-MESC. Several novel findings in the in vitro LD-MESC model are summarized below.

Continuous hypoxia, and 14 day completely under hypoxic conditions employing a single hybrid medium formulation (containing VEGF, EGF, IGF, FGF and 2% KO serum to activate signaling) without formal instructive factors were employed, resulting hPSC-hepatoblast (HB) induction from endoderm. This 14 day culture applies principles of differentiation, and is unique in the field.

An organoid compaction step was incorporated to show organoid compaction and mesoderm modeling, after which organoids were transferred to a matrigel droplet to model the LD-MSC. The organoid modeled the LD, and the MG and endogenous hPSC-derived mesoderm (MES) modeled the MES, from day 15-18 of culture.

Induction of DE followed by exposure to the medium formulation (Hepatic+Mesenchymal medium), enabled induction of FOXA2(+), ALB(+), AFP(+), PROX1(+), CDX2(−) hepatoblasts compared to a conventional protocol, with evidence of a transient CD31(+) (endothelial) population by day 14. Day 15-18, continuous treatment with H+M medium resulted in extensive collective migration, with evidence of branching and bifurcation, thin, radially directed, finger-like 3D outgrowths that expressed liver markers, and enhanced liver and hepato-mesenchymal differentiation based on gene (FOXA2(+), HNF4A(+), PROX1(+), TBX3(+), PDX1(+), FOXF1(+), RUNX2(+)), protein (AFP(+), ALB(+), TBX3(+), CD31(+), SMA(+)) expression, and low (immature) functional analysis (urea and ALB secretion). Low functioning, immature cells were desired, since early liver organogenesis was being investigated.

Based on over 30 signaling pathways we observed, a chemical screen was designed over 8 pathways over 24 conditions. It was identified that Hippo signaling and VEGFR2 signaling were positive hits which inhibited organoid growth and migration. This was consistent with signaling pathways identified in our bioinformatics analysis. Analysis of transcription was performed which validated that Hippo pathway (YAP-TAZ/TEAD) signaling likely plays a role.

To test whether the day 15 cells have a hepatomesenchymal phenotype, culture was performed on a bioengineered microdevice which demonstrated tissue formation and tension suggesting a hepatomesenchymal phenotype.

Thus, the following more clearly defines events of early liver organogenesis, leading to knowledge about the transcriptome and the niche for E8.5, 9.5, 10.5. Next, extensive bioinformatic analysis of scRNA-seq data of murine liver organogenesis was performed, for which certain techniques were developed, resulting in several novel findings of the process. This knowledge was used to hypothesize that the LD-MESC functions as a signaling center in early liver organogenesis. After performing an in vivo model that demonstrated hypoxic tissue growth, principles of early liver organogenesis culture and for designing the hLD-MESC with hPSC were formulated. The protocol has 3 stages (hPSC-hepatoblast induction), monolayer, organoid formation, and migration. Induction of hPSC-hepatoblasts incorporated several principles of culture. After forming organoids, extensive collective migration and quantifiable evidence of growth, evidence of rapid Albumin activation were observed. Based on the scRNA-seq findings, signaling pathways were screened that effect growth and migration in this model. It was found that hippo pathway (YAP-TAZ) mediates growth, which agreed with the bioinformatics data. Finally, to demonstrate the emergence of hepatomesenchyme, a novel functional bioengineered device was employed which established functional evidence of a mesenchymal phenotype.

### EXAMPLE 2

Organoids bearing human stem cell-derived progenitors enable basic and applied investigation of organogenesis in a wide range of epithelial tissues. During liver organogenesis (LO), E9.5 collectively migrating hepatoblasts (MHs) arise from the E9.0 liver diverticulum (LD) and directly penetrate the surrounding mesoderm (MES) tissue, forming cell strands that link migration, differentiation, and growth. Currently, human pluripotent stem cell (hPSC) organoid protocols model the E10.5 liver bud and forward differentiation, but not the LD or the LD-derived MHs, in spite of their significance. In fact, the transcriptome underlying MHs, the niche that drives their migration, and methods to induce them from hPSC remain key questions.

In this example, bioinformatics analysis of single cell RNA-seq data, in vivo transplantation, and in vitro hPSC differentiation with organoid formation, microscopy, gene and protein expression, small molecule inhibitor screening of growth, and organoid culture were performed in bioengineered devices to assess tissue tension.

The in depth bioinformatic analysis of early murine LO demonstrates pathway up-regulation of an unexpected wide array of soluble signaling factors, as well as cell cycle, chromatin modification, and metabolic reprogramming, in addition to a widespread cell stress-response. These findings led to the hypothesis that the LD and MES tissue form a tissue complex (LD-MESC) that drives MH induction. Using this LD-MESC concept, an in vivo transplant system was designed, as well as a three-step in vitro protocol for inducing hPSC-derived MHs, both of which recapitulate liver growth, morphogenesis, differentiation. It was shown that Hippo signaling pathway, in agreement with murine MH data, mediates migration and growth of hPSC-MH in vitro. These data substantiate the LD-MESC model developed here, and directly address key challenges facing liver regenerative medicine.

Bioinformatics, in vitro, and in vivo data all support the concept that the LD-MESC initiates LO. This concept can be used to change protocols to emphasize linking of migration, growth, with differentiation. Modeling epithelial collective migration for LO bolsters not only organogenesis studies of alternate endodermal organs, but also in vivo transplantation efforts, and facilitates employing migrating organoids to therapeutically target human tumor migration/metastasis.

LO establishes liver mass, microarchitecture, and numerous liver-specific functions. Based on these principles, current methods for modeling LO with hPSC include: 1) Directed differentiation protocols, 2) Organoids with exogenously added mesoderm-derived tissues (MES), 3) Organoids with endogenous MES, 4) Assembloids of interacting organoids and 5) Synthetic biology-based organoids. Early LO (eLO) is a stage during which early growth, differentiation, and migration are coordinated. In eLO, the E9.0 liver diverticulum (LD), an out pocketing of tissue with only ˜1500 cells, amazingly, expands by ˜10²-fold by E10 and by 10³-fold by E11.5. The LD transitions to the E10.5 liver bud (LB) bearing the microarchitecture for forming primitive sinusoids. Next, the LD initiates outward (ventral) three-dimensional (3D) collective cell migration (CCM) (E9.5). Next, hepatoblasts (HBs) that migrate (MHs) self-organize into hepatic cords, branching into adherent migrating cell stands within surrounding MES tissue. This extensive morphogenesis is accompanied by highly coordinated, critical transitions in gene expression to form HBs. Here, developmental gene regulatory networks (GRN) composed of master transcription factors (TFs), including FOXA2, HNF4A, PROX1, and TBX3, help initiate and maintain hepatic fate, boosting albumin (ALB) gene transcription and triggering CCM. In summary, eLO occurs starts with the LD followed by highly impactful events.

This example establishes the significance of the eLO using bioinformatic analysis, in vivo, and in vitro studies. It was hypothesized that the LD and surrounding MES form a tissue complex (LD-MESC) that initiates and coordinates eLO. This bioinformatics analysis employed data from recent scRNA-seq studies that have provided several valuable insights into early LO and support the concept of the LD-MESC. Based on the bioinformatics analysis and on in vivo transplantation data, a novel hPSC protocol is developed that mimics the MH population. Extensive in vitro imaging and characterization of the MH population is provided, as well as mechanistic analysis which demonstrates pathways that control hLD growth and CCM.

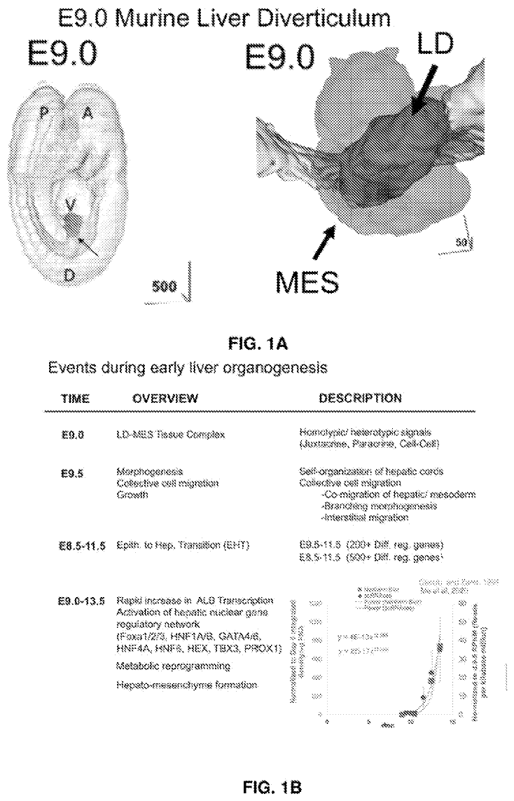

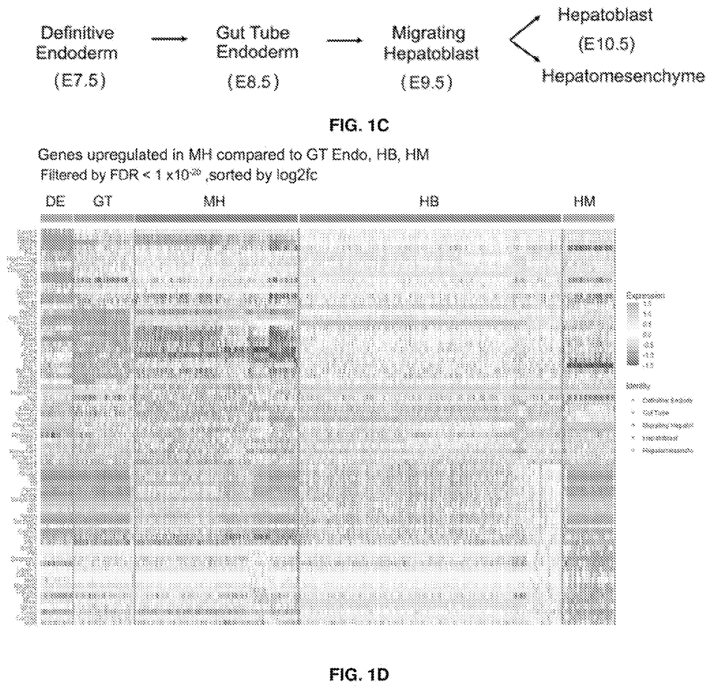

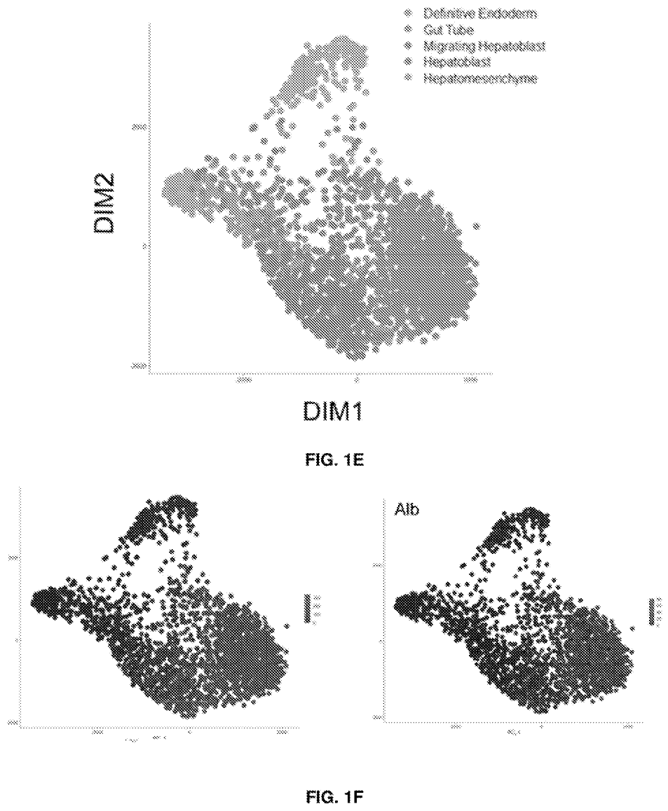

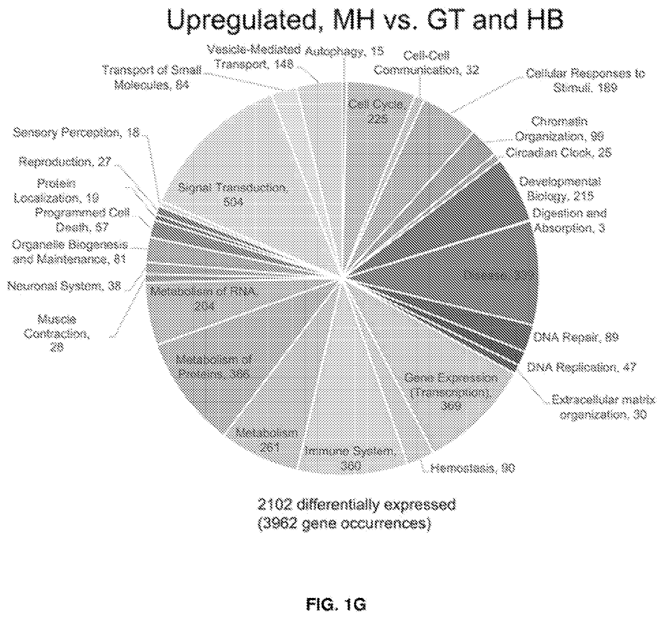

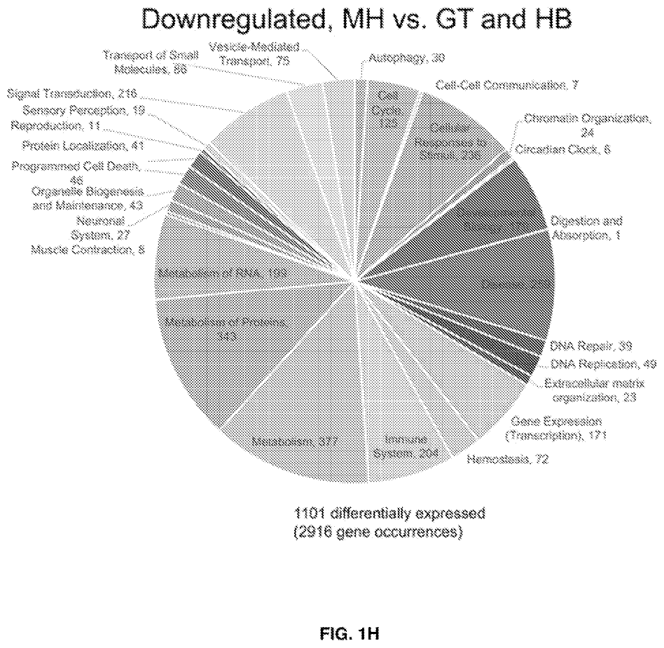

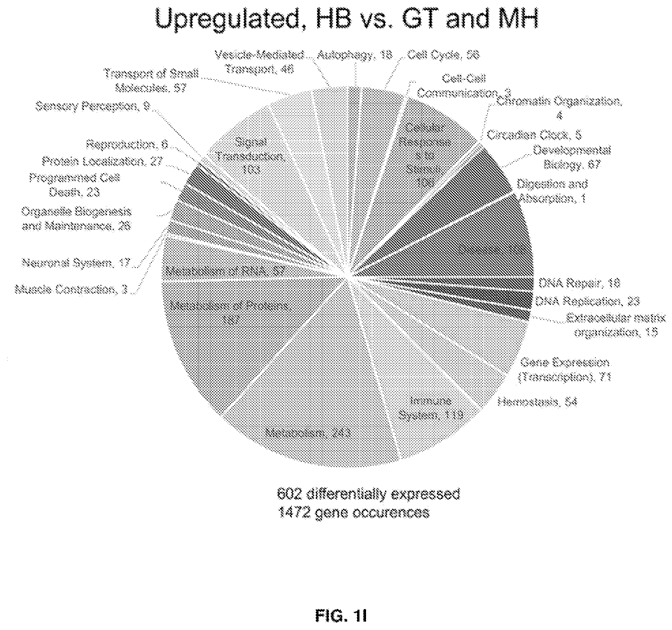

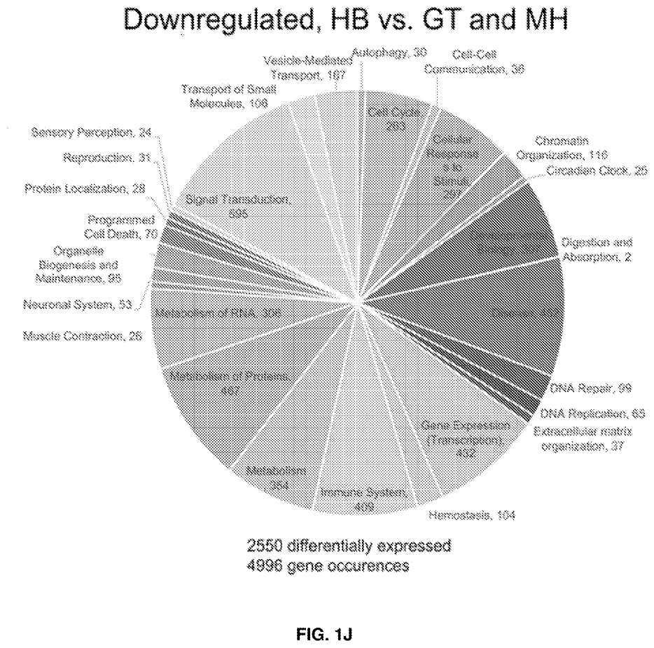

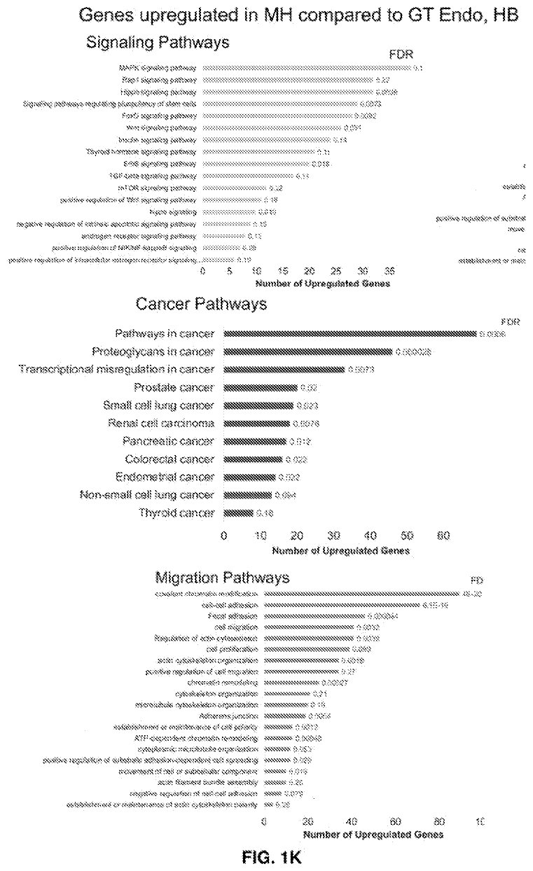

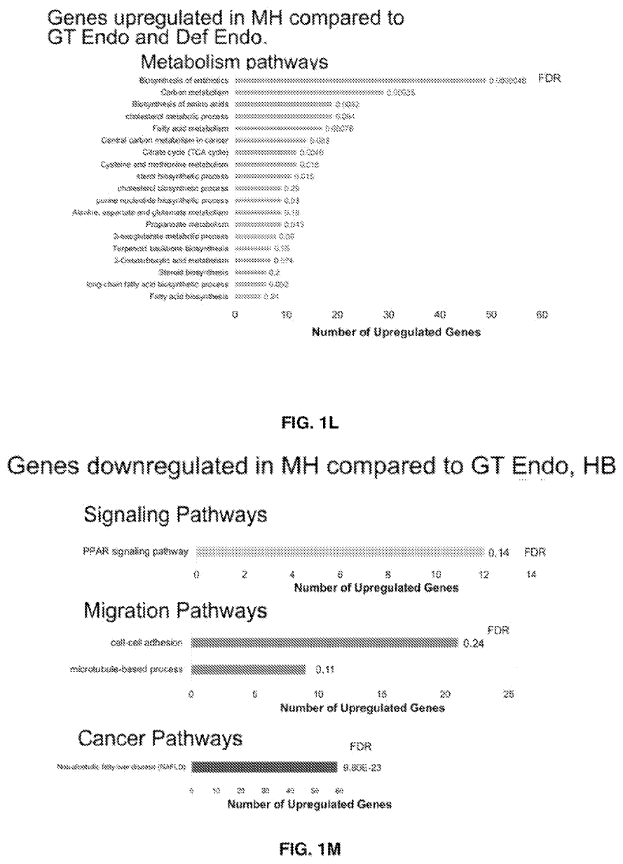

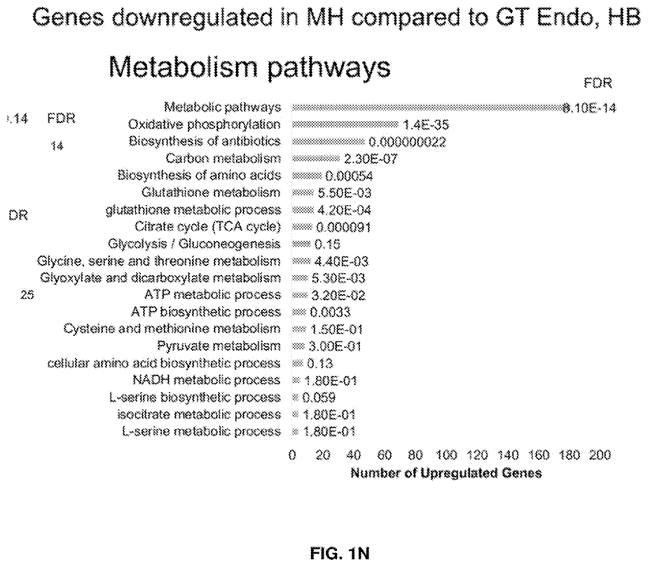

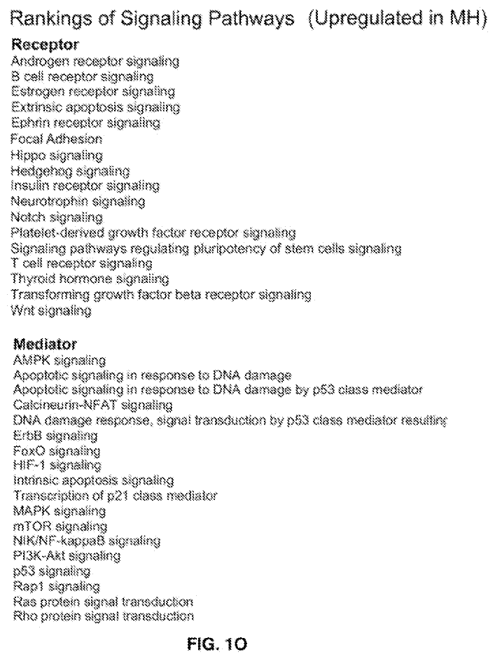

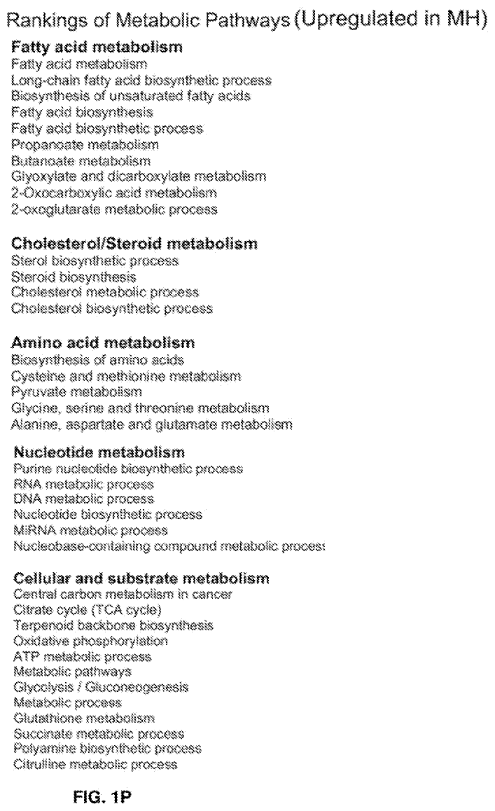

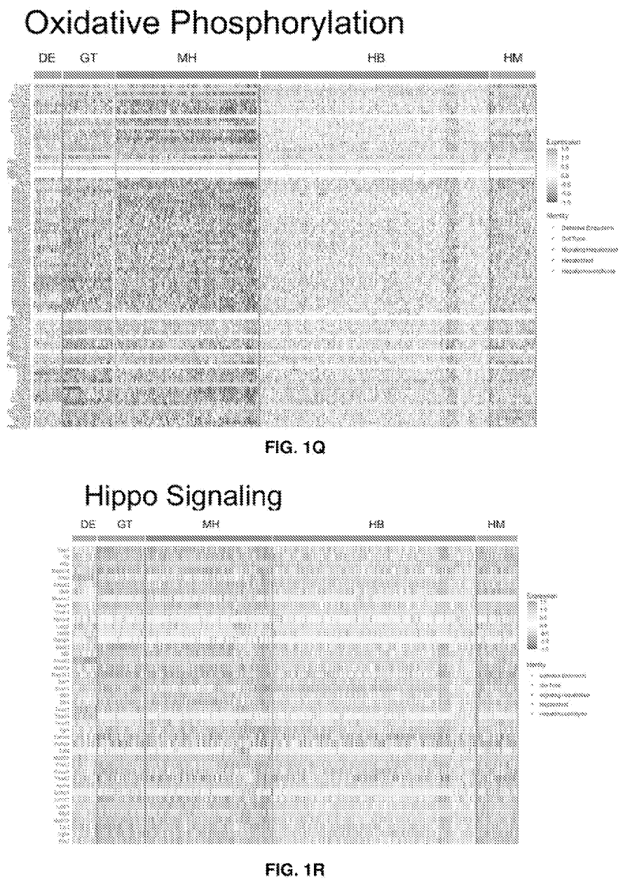

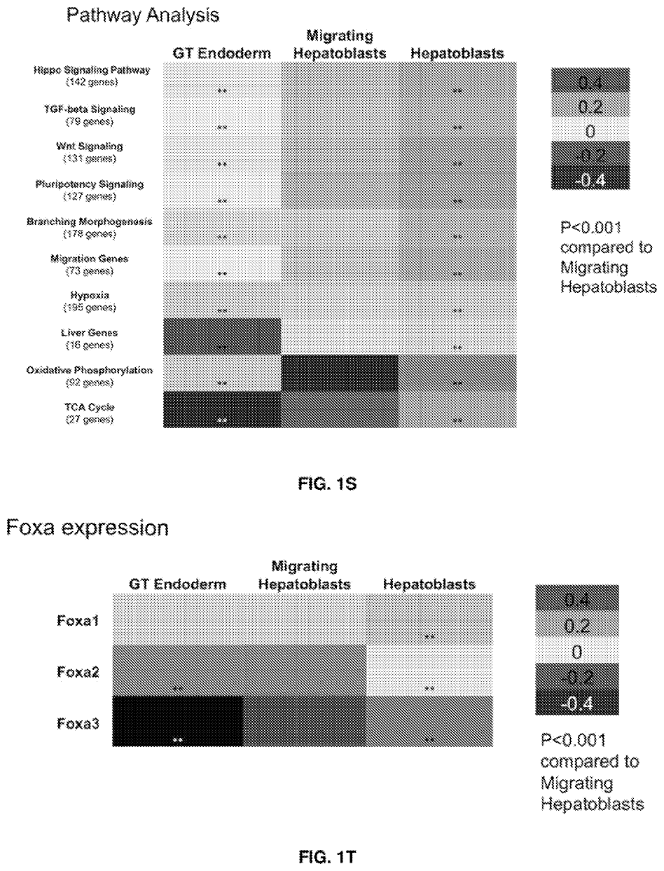

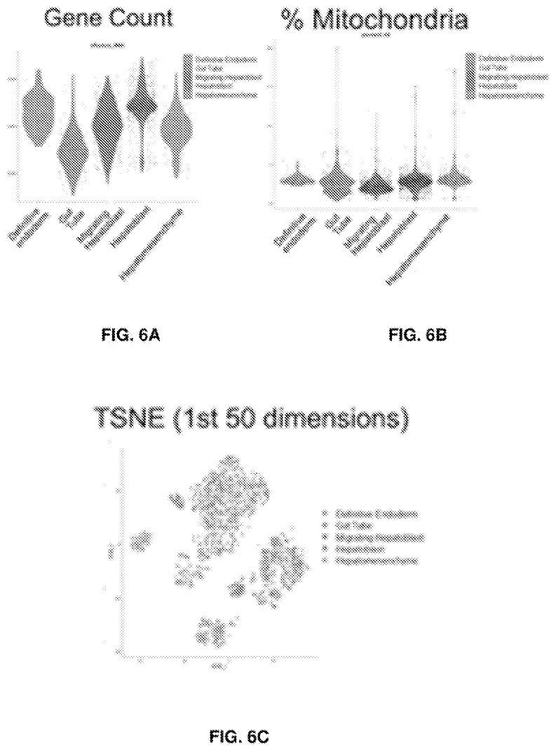

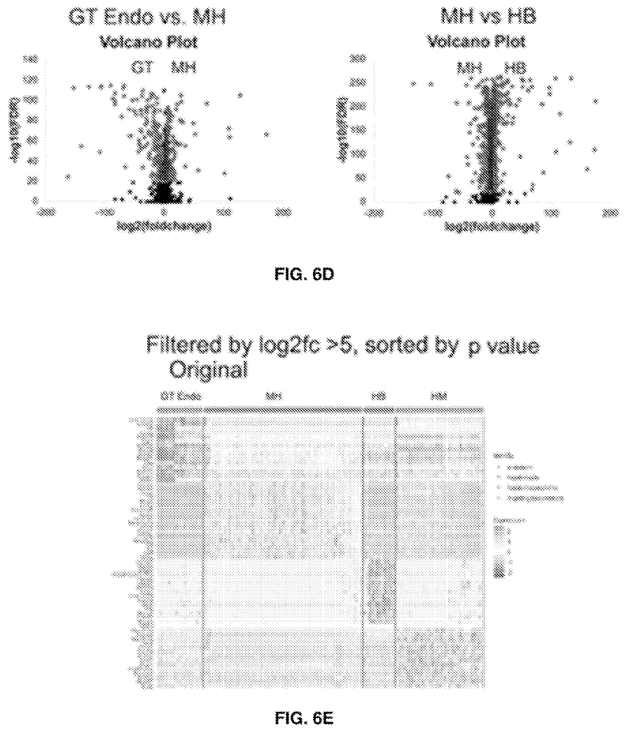

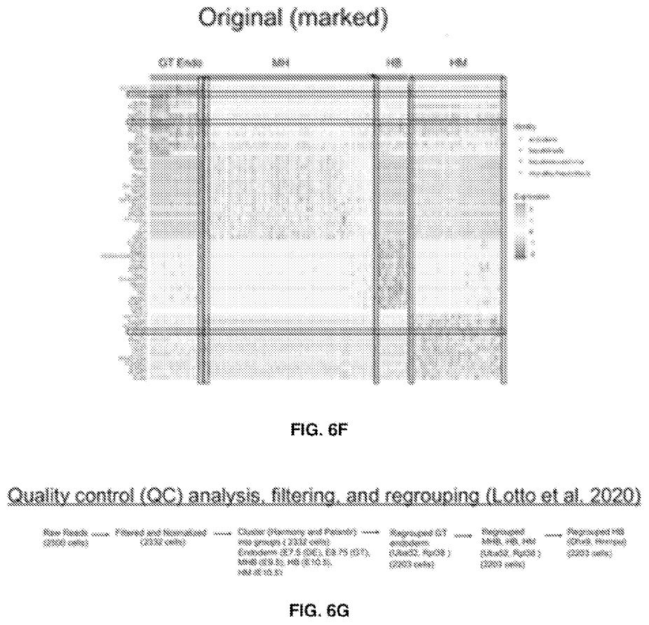

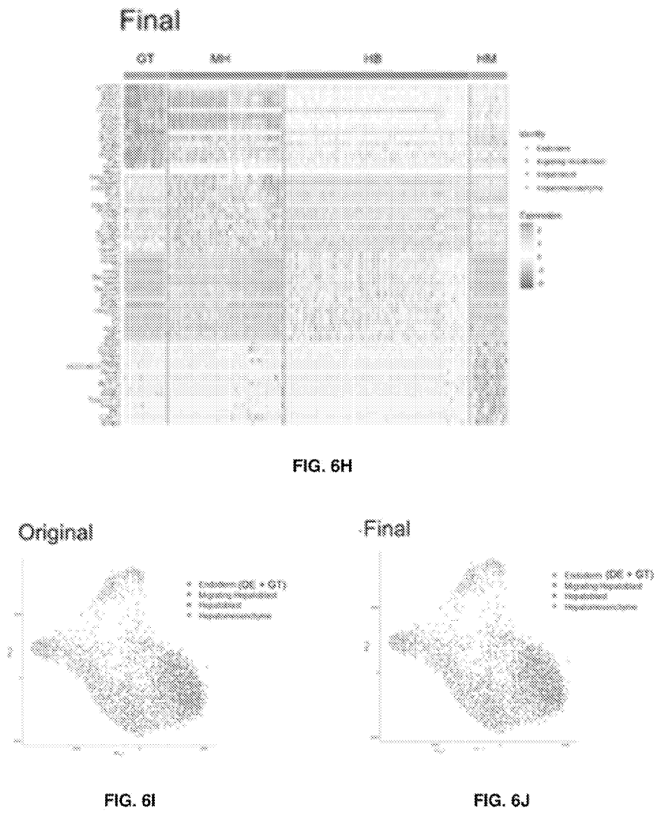

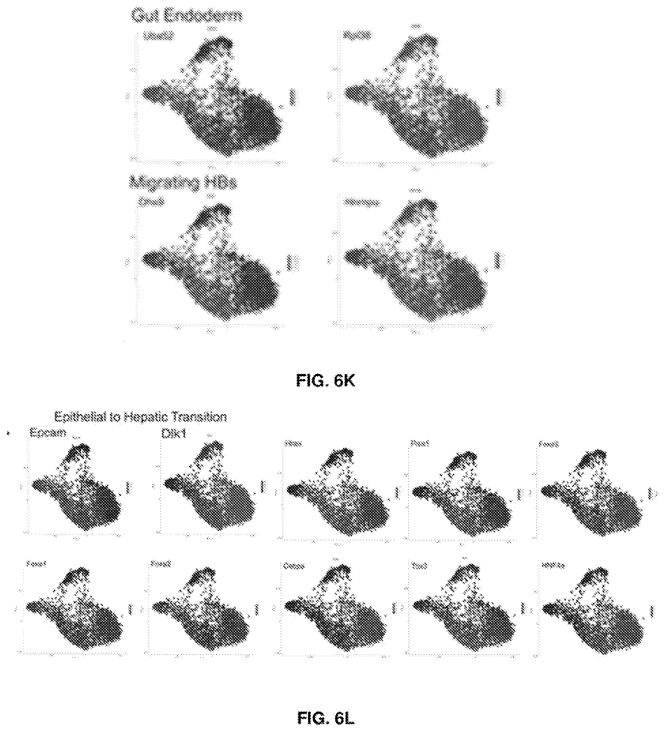

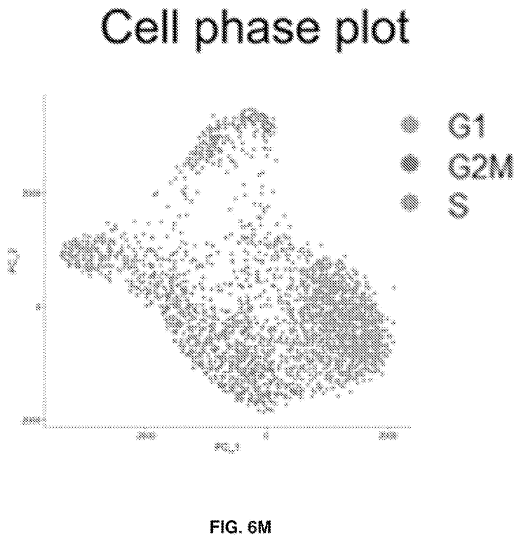

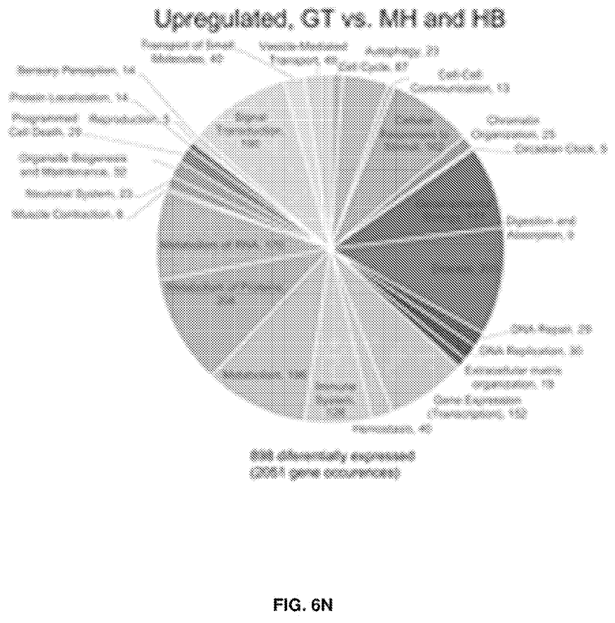

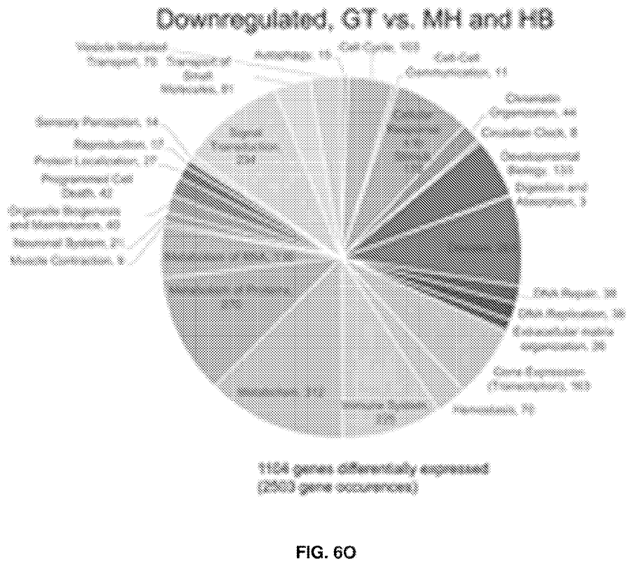

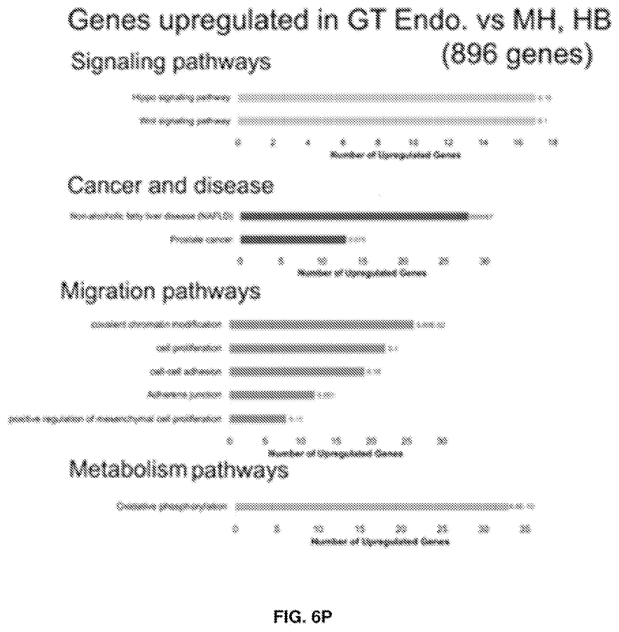

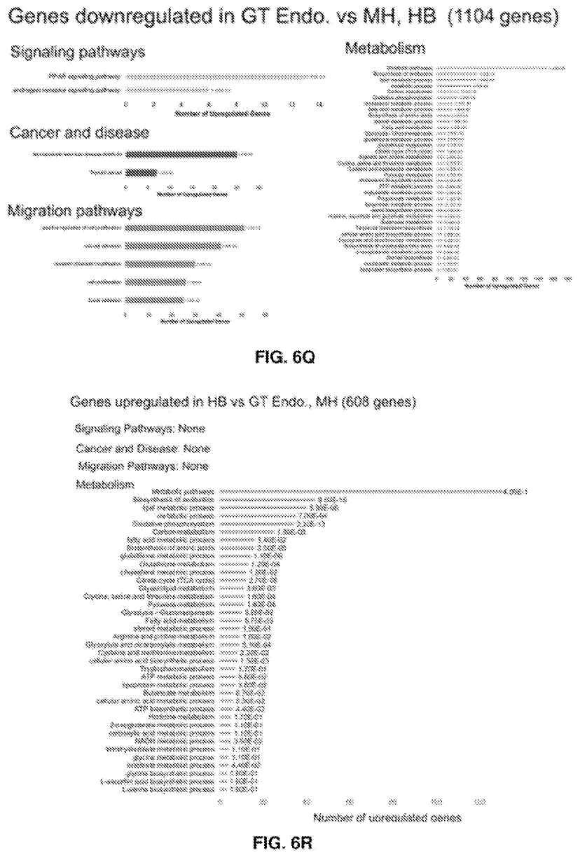

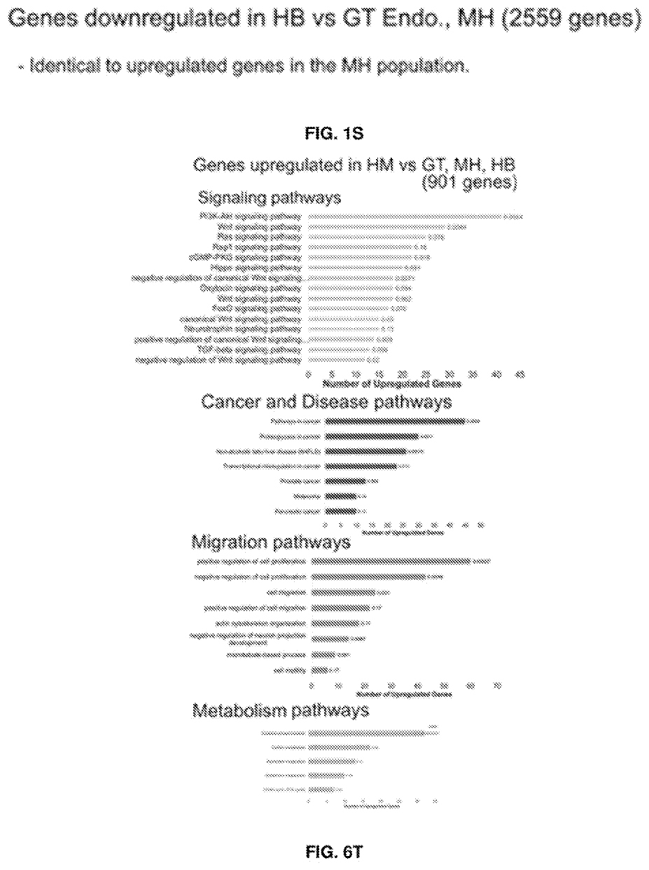

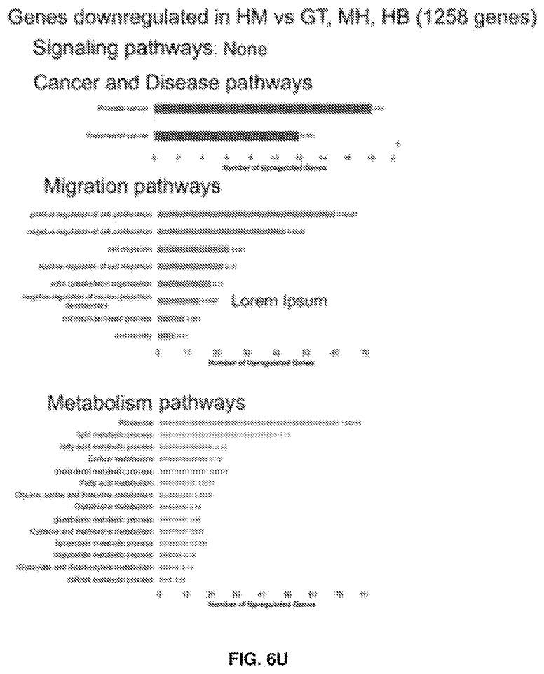

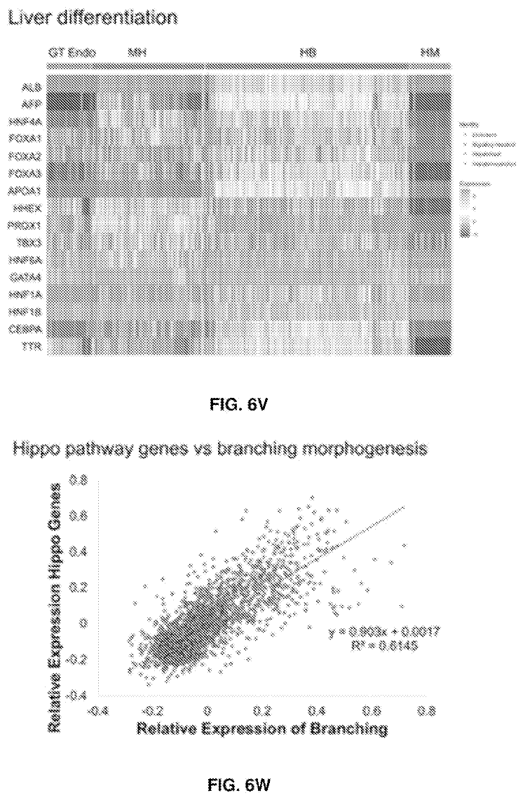

Transcriptome analysis highlights coordinated up-regulation of signaling, CCM, and metabolic pathways in E9.5 migrating hepatoblasts. To elucidate the factors that drive the E9.0 LD-MESC to trigger eLO (FIG. 1A-C), extensive bioinformatic analysis of E9.5 MHs was performed. First analyzed was the increase in ALB transcription which showed an exponential increase (FIG. 1B). It was assumed that definitive endoderm (DE, E7.5) gives rise to gut tube (GT, E8.5), then to MH (E9.5), which then gives rise to either HB (E10.5), or hepato-mesenchyme (HM) (E10.5) (FIG. 1C). Initial quality control (FIG. 6A-D) demonstrated clear differences between the GT, MH, and HB conditions. However, re-clustering (FIG. 6E-F) was performed. See Methods-Re-clustering). To understand key regulated pathways in the MH population, up- and down-regulated genes were analyzed in GT, MH, and HB cells (initially the HM was removed), and we three software analysis-based approaches (REACTOME, DAVID, and ENRICHR) were employed which were validated (See Methods-Pathway validation, FIG. 1). With the set of differentially expressed genes (DEG), first performed was REACTOME analysis with pie charts for the GT, MH, and HB clusters (log2fc>0.5, FDR<0.05) (FIGS. 1G-J, FIGS. 6N-6O). Each cell cluster was compared to the other two. Pie chart analysis for the GT population showed pathways for 898 DEG (FIG. 6N-O). The MH pie chart depicting upregulated gene groups (2102 DEG), demonstrated that Signal Transduction. Transcription, Immune system, Cell Cycle, and Chromatin Organization gene groups were highest (FIG. 1G). For the downregulated MH gene groups (2916 DEG), it was observed that Metabolism, Metabolism of proteins, Cellular Response to Stimulus, Metabolism of RNA, were highest (FIG. 1H). The HB upregulated pic chart (602 DEG), demonstrated Metabolism, and Metabolism of Proteins were higher in the HB compared to the GT and MH lineages (FIG. 1I). For the downregulated HB gene groups (2550 DEG), it was observed that Signal Transduction, Disease, Developmental Biology. Transcription, Cell Cycle, and Chromatin Organization, were highest (FIG. 1J). The data establishes that signal transduction, transcription, cell cycle, and chromatin organization are critical within the E9.5 MH population, and are downregulated in the E10.5 HB population. This suggests the existence of a transient niche and a dynamic up-regulated and down-regulated MH-specific transcriptome, suggesting a robust switch in differentiation and phenotype.

Next, it was hypothesized that up-regulation of signaling pathways is associated with changes in transcription, chromatin organization, growth (cell cycle), metabolic changes, and CCM in the MH population. Using DAVID biological process, several signaling pathways were identified, significantly upregulated in MH, including MAPK, Rap 1, Hippo, Pluripotency, Insulin, FoxO, ErbB, TGF-β, mTOR, Wnt, and NF-κB (FIG. 1K). Also identified were cancer/disease pathways which were up-regulated (prostate, lung, renal cell, pancreatic, colorectal, endometrial, thyroid) (FIG. 1K). Interestingly, most of these cell types are derived from DE. CCM pathways were analyzed in the MH, and it was found that up-regulation of Covalent Chromatin Modification, Cell-Cell adhesion, Focal Adhesion, Cell Migration, Regulation of Actin Cytoskeleton, and Cell Proliferation. This data regarding Cell-Cell adhesion supports that MHs are undergoing CCM (FIG. 1K). Metabolic pathways were analyzed, and it was found that up-regulation of lipid metabolism, DNA metabolic processes, and RNA metabolic processes (FIG. 1L), and biosynthesis of amino acids, nucleotides, cholesterol, steroid, and fatty acid metabolism (FIG. 1O). Also analyzed were down-regulated pathways (FIG. 1M, 1N, 1P), which included a down-regulation of CCM pathways and of both TCA cycle and oxidative phosphorylation genes. Also examined were up-regulated and down-regulated pathways in the GT, MH, HB, and HM populations (FIGS. 6P-U). Compared to the MH population, the HM populations also exhibit up-regulated CCM genes, and signaling pathways, but down-regulated liver differentiation genes.

To further substantiate the findings with DAVID, a ranking approach was developed for analyzing individual pathways based upon both the adjusted FDR level (0.3), and frequency of up-regulation (or down-regulation) in the sixteen possible comparisons between MH and the other (DE, GT, HB, and HM) cell populations. A list of 35 signaling/cognate receptor pathways was found, divided to obtain a list of 17 upregulated signaling pathways and 18 upregulated intracellular mediators (FIG. 1O). Similar analysis were performed for metabolic signaling pathways (FIG. 1P). One-third of metabolic pathways identified were lipid metabolism (combining Fatty acid and Cholesterol metabolism), with increased glyoxylate and dicarboxylate, 2-oxyglutarate, and 2-oxocarboxylic acid process, metabolites linked with fatty acid metabolism, hypoxia signaling and chromatin modification, and cell proliferation, respectively. This analysis was validated by performing analysis with ENRICHR (Table 7). Taken together, this ranking approach identified numerous up-regulated signaling and metabolic pathways.

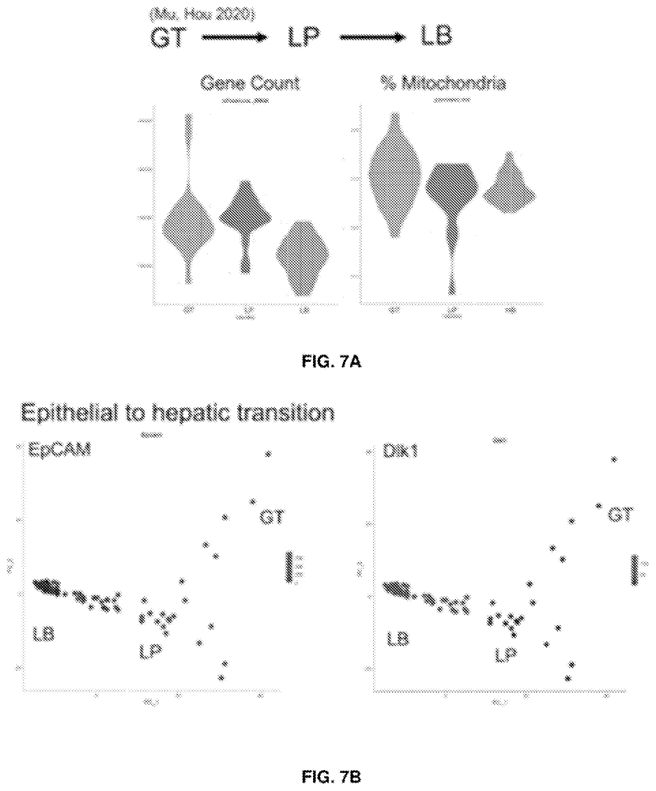

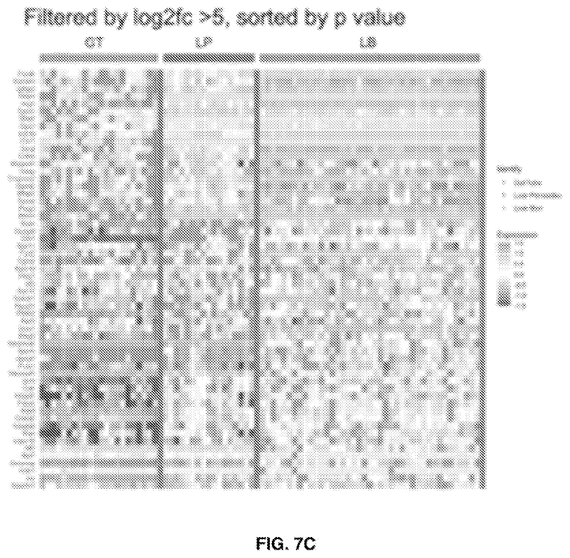

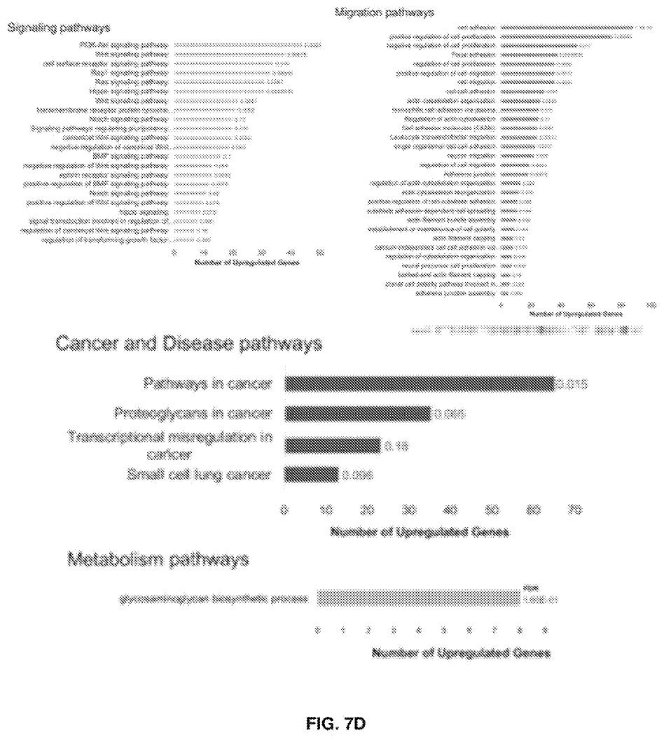

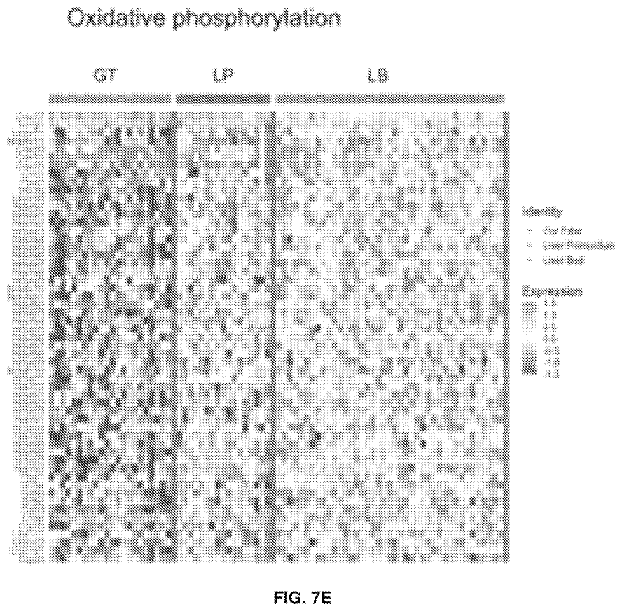

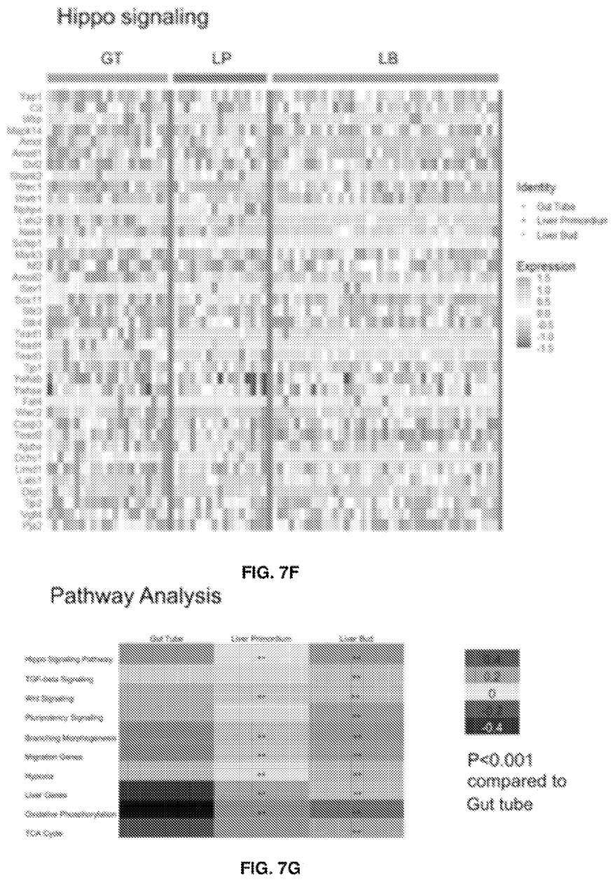

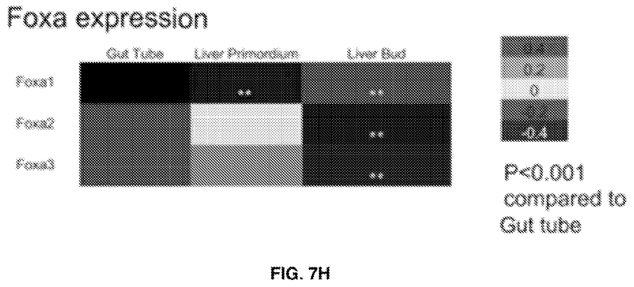

To further delineate the MH phenotype. focused heat map analysis was performed on signaling (Hippo signaling, TGF-β, Wnt, Pluripotency), Branching Morphogenesis, Migration, Hypoxia, Metabolism (Oxidative Phosphorylation, TCA cycle), and Master Liver Transcription Factors, with examples shown (FIG. 1Q-R, FIG. 6V). A significant coordination of gene expression in the MH population was observed, with all pathways upregulated except Liver differentiation, Oxidative phosphorylation. and TCA cycle genes (FIG. 1S, Table 7). with mixed expression of Master Liver Transcription factors. It was hypothesized that FOXA1/2/3 may correlate with the coordinated changes in pathways. due to their role in liver differentiation and metabolism. Interestingly. down-regulation of FOXA2 correlated with coordinated pathway changes (FIG. 1T), suggesting FOXA2 expression may play a role. The main findings of the MH population were analyzed by analyzing a second scRNA-seq study of liver development (FIG. 7A-H).

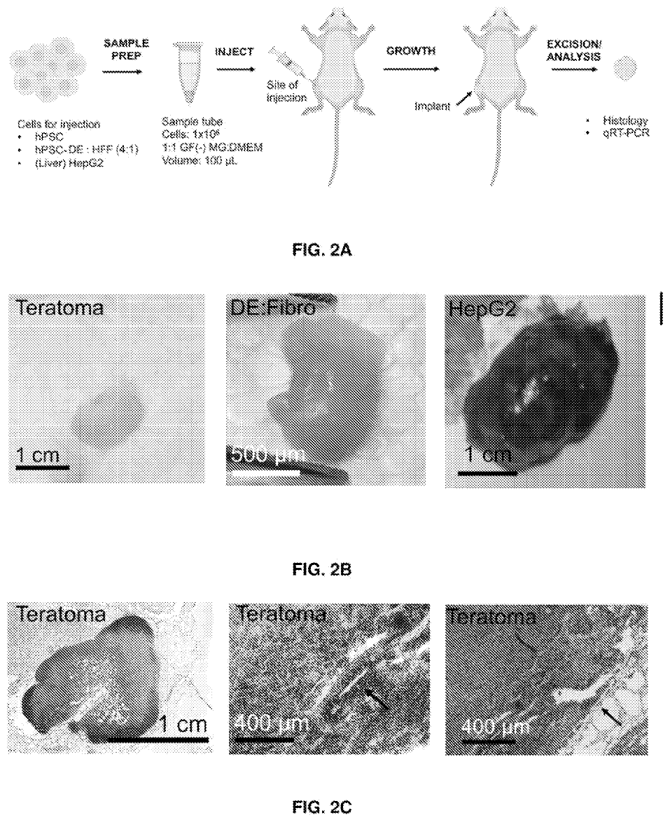

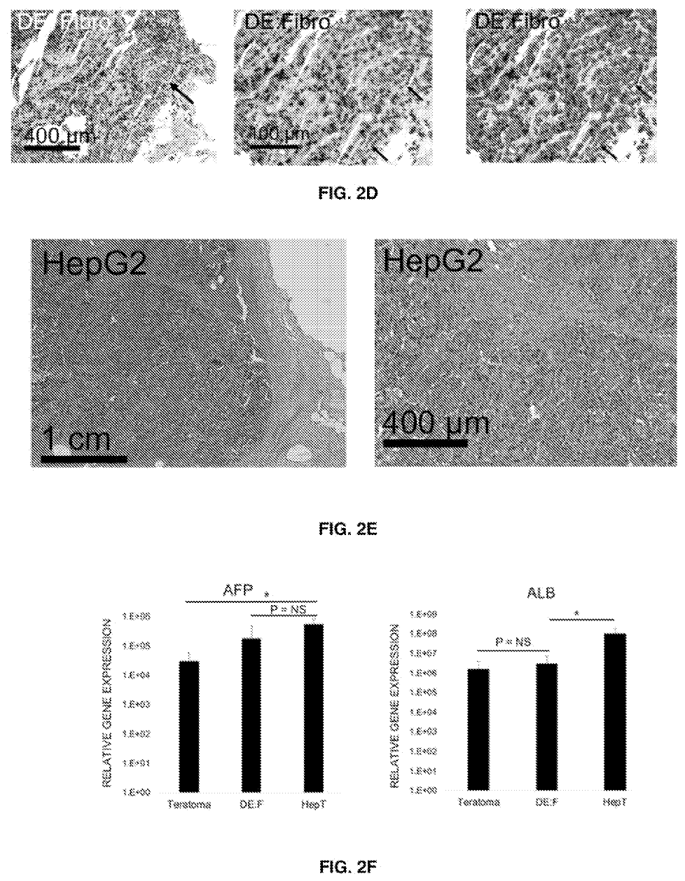

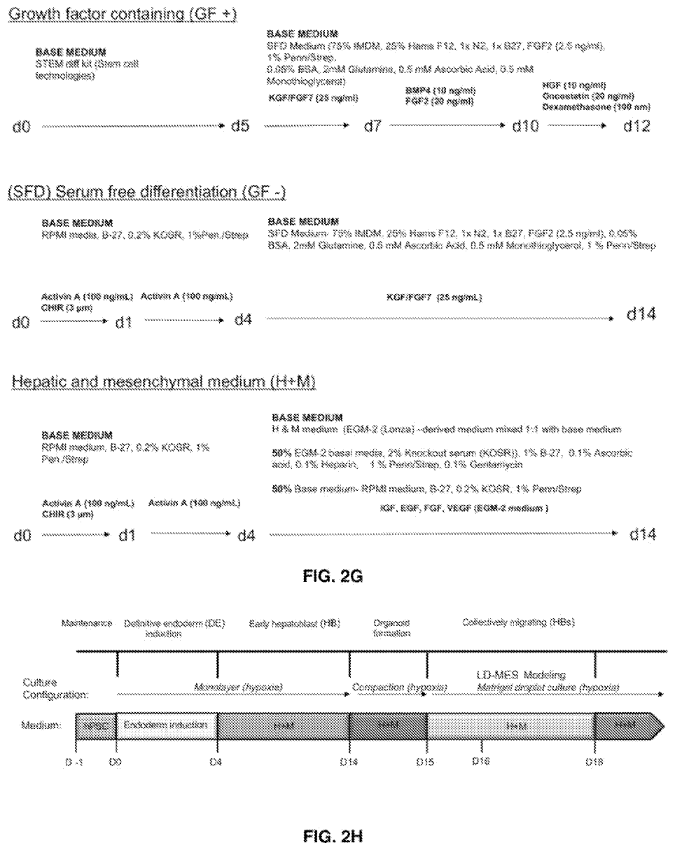

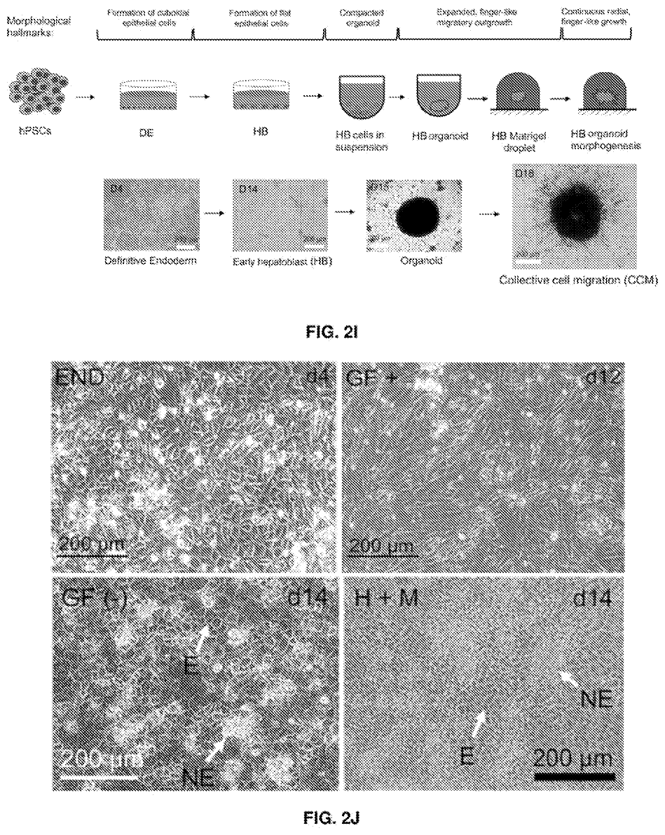

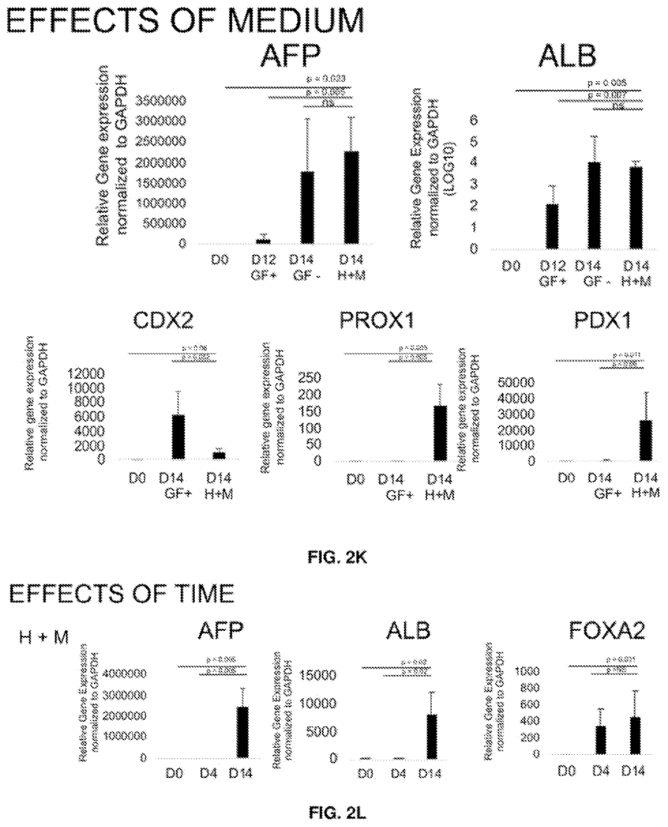

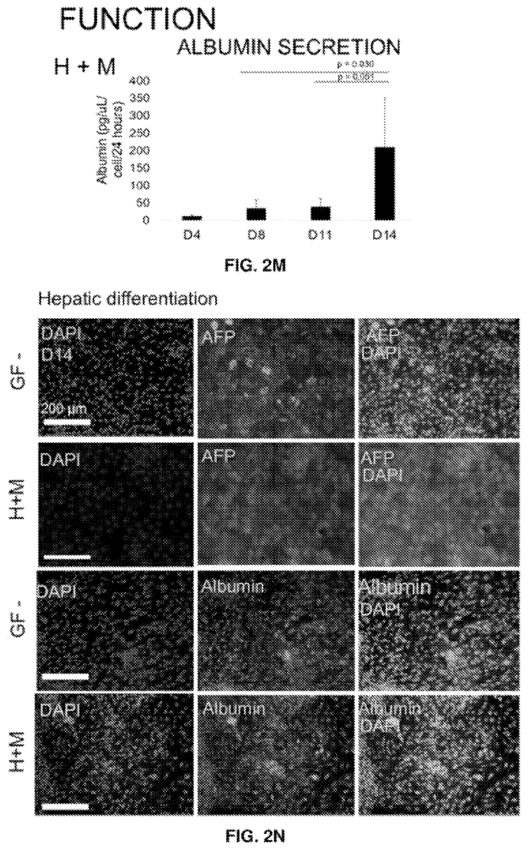

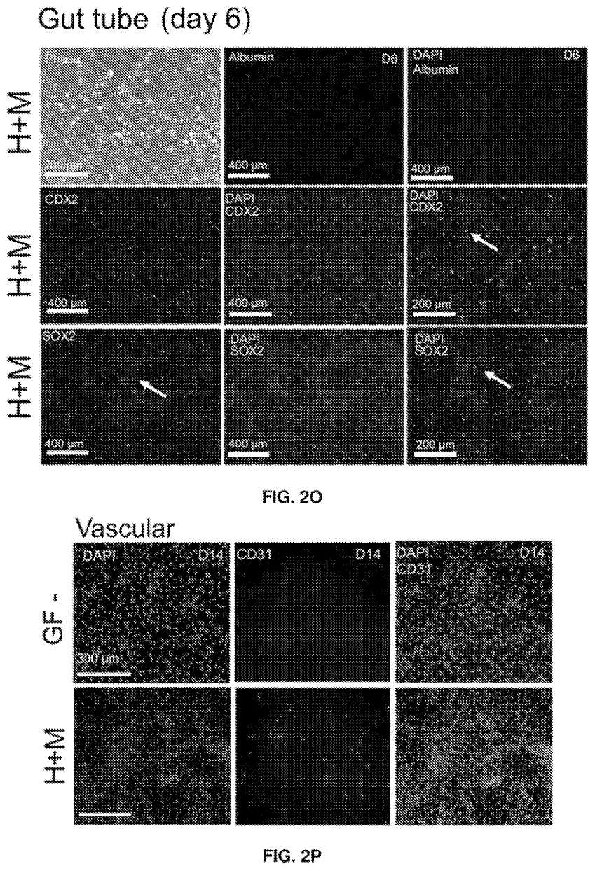

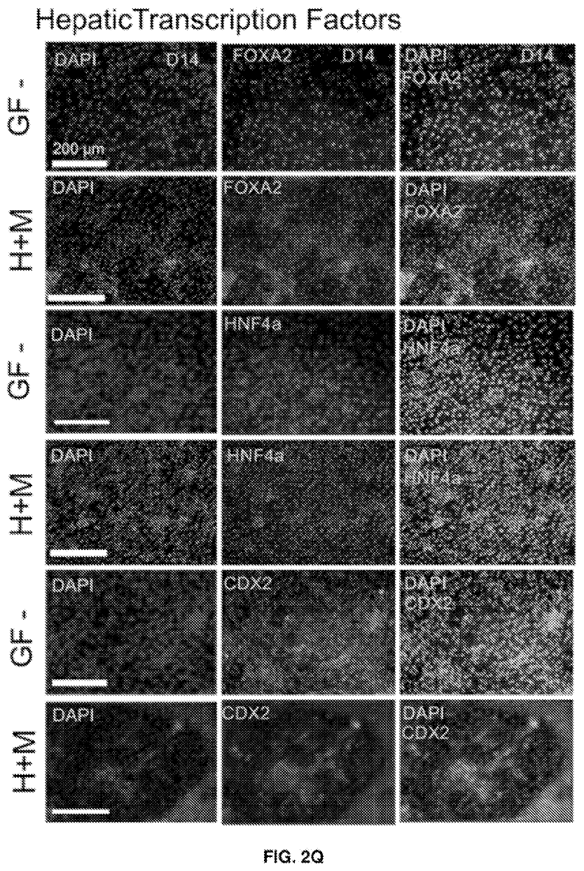

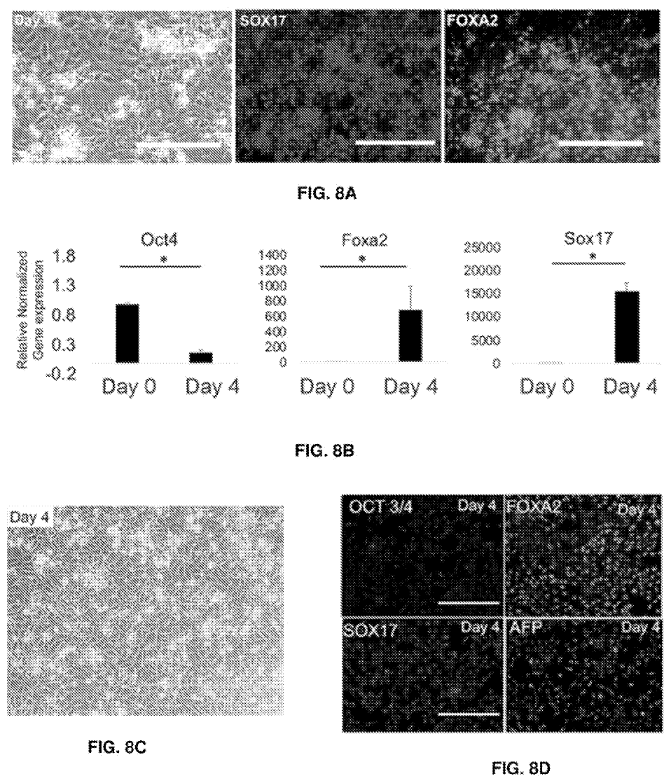

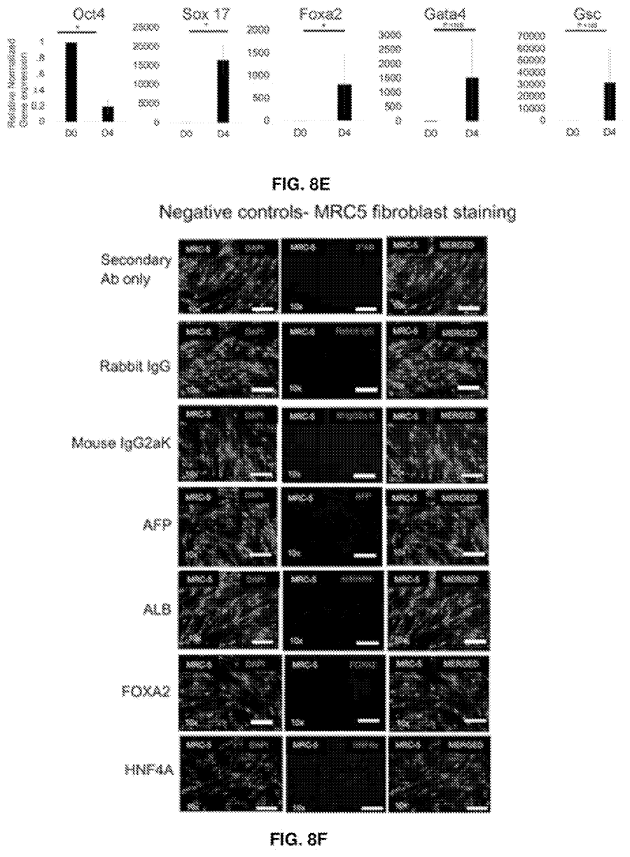

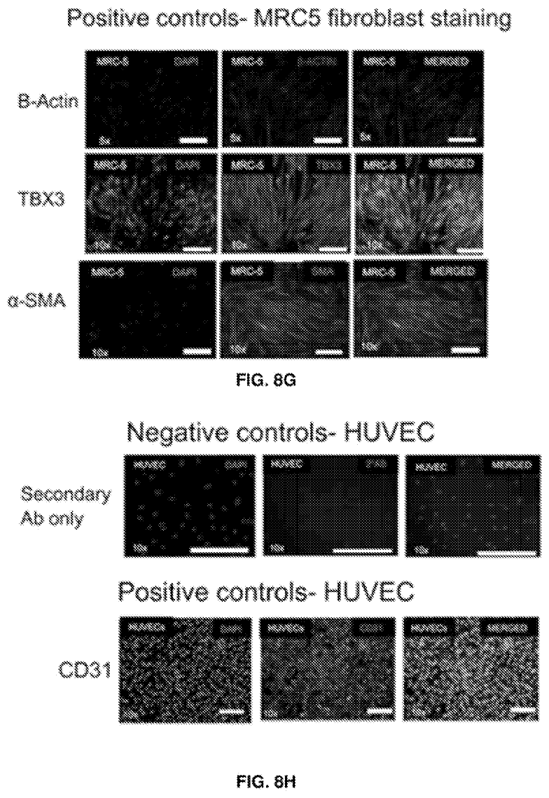

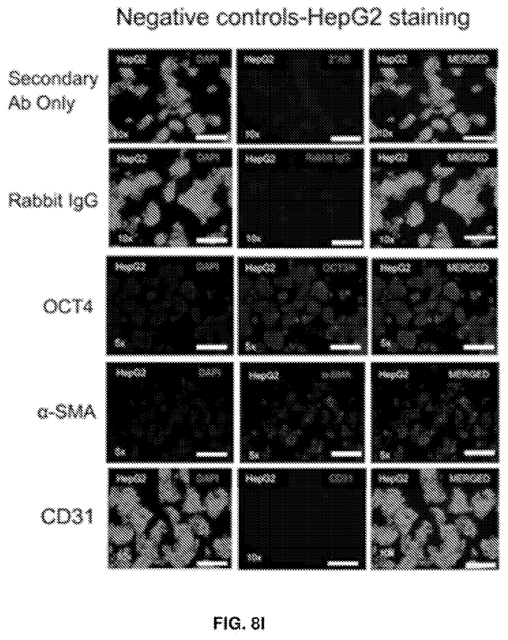

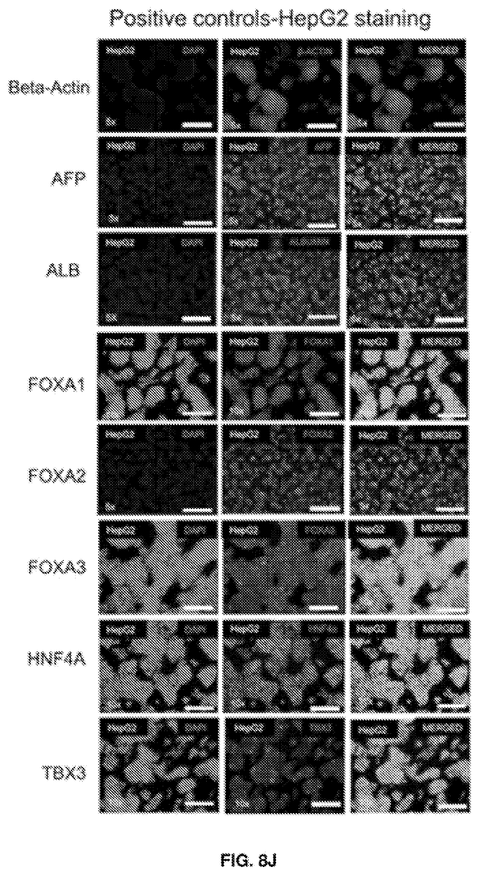

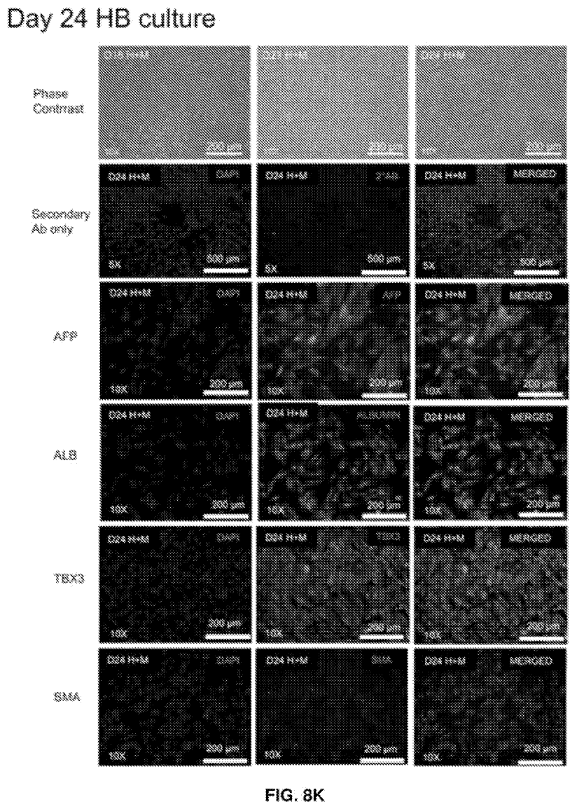

Design of a novel transplant model that supports the role for LD-MESC in early LO Since changes in the E9.5 MH population were triggered in the E9.0 LD-MESC. the hLD-MESC with an in vivo system was first modeled. Murine LD-MESC growth is characterized by hypoxic growth. morphogenesis/CCM within MES tissues, and rapid ALB transcription. To model this. hPSC-derived DE was employed. which presumably would form HE in vivo and human foreskin fibroblasts (HFF) to model the MESC. Two controls were empolyed, hPSC alone, and a hepatoblastoma-derived (HepG2) cell line. in addition to the hPSC-DE (FIG. 8C-E) mixed with HFF. Tissues were transplanted subcutaneously to preserve hypoxic conditions (FIG. 2A). All three conditions generated tissues in vivo after 4 weeks (FIG. 2B). hPSC alone formed teratoma-like tissues, as hematoxylin and cosin (H+E) staining highlighted the germ layers (FIG. 2C, left, middle, right panels). The hPSC-DE mixed with HFF and transplanted demonstrated that DE-derived cells formed cords of cells within the fibroblast mass. indicative of hepatic cord morphogenesis, but with no apparent blood vessels (FIG. 2D, left and middle panel, right panel (highlighted by image segmentation)). Transplanted human HepG2 liver cells resulted in a homogeneous liver tissue architecture (FIG. 2E, left and right panels). qRT-PCR showed that the DE:HFF condition had comparable AFP expression to the hepatoma control (FIG. 2F, left panel), and the same levels of ALB as the teratoma control (FIG. 2F, right panel). Therefore, the DE:HFF condition expressed high levels of ALB, and these values (˜10⁶) defined the upper limit of ALB transcription, demonstrating maturity. Importantly, both the teratoma and the DE:HFF condition were mixed samples that contained RNA of other contaminating cell types, and that likely the hepatic-specific RNA values were indeed higher. It was determined that the in vivo growth rate by estimating cell size initially and after 4 weeks, and compared to in vivo growth for 4 weeks, a 40-fold increase in volume was observed compared to in vivo eLO. Nonetheless, the data suggests that the hLD-MESC model exhibits hypoxic exponential growth, morphogenesis and CCM within MES tissue, and rapid ALB transcription, events that occur in the murine LD-MESC (FIG. 2F, right panel).

Design of a novel and reproducible protocol for hPSC-HB induction with continuous mesenchymal signaling. Based upon the analysis of eLO and existing hepatic protocols, a novel hepatic induction protocol was developed that involves continuous hypoxia, an absence of maturation factors, a reliance on spontaneous HB formation, and a single medium formulation. After hPSC-DE induction (FIG. 8C-E), a control, the GF(+) condition, was compared with the hepatic and mesenchymal (H+M) condition (1.1% KO serum), and a serum-free formulation (SFD), the GF(−) condition (FIG. 2G). The overall protocol (FIG. 2H-I) was applied to multiple embryonic stem cells (hESC) and hiPSC cell lines were tested with similar results. Day 4 hPSC-DE demonstrated cuboidal cells with bright cell borders (FIG. 2J, top, left). In the control, GF(+), condition, a more elongated morphology was observed by day 12 (FIG. 2J, top, right), whereas more cuboidal epithelial morphology was observed in the GF(−) and H+M conditions (FIG. 2J, bottom left, bottom right). In the H+M condition, both epithelial (E) and non-epithelial (NE) elements were observed.

The effects of medium on gene expression were first examined. Although elevated levels of AFP and ALB (FIG. 2K) were observed, PROX1 was significantly upregulated in the H+M compared to GF(−) condition, which is normally expressed in both hepatic and extrahepatic biliary duct progenitors. Further, CDX2 was significantly downregulated in the H+M compared to GF(−) condition, and is expressed in hindgut and intestine (FIG. 2K). Based on this data, as well as time course data (FIG. 2L), H+M was employed in further experiments. ELISA analysis demonstrated a steady increase in ALB secretion from day 4 to 14, although ALB secretion was low compared to human functional HEPs in a stable culture system (FIG. 2M). Next, immunoanalysis of the GF(−) and H+M conditions (FIG. 2N-Q, FIG. 8F-J) was performed. It was observed that high AFP expression and low/intermediate levels of ALB expression for both conditions (FIG. 2N). In the day 6 H+M condition, it was observed that no ALB expression, low levels of CDX2 expression (mid and hindgut), and SOX2 (foregut), consistent with ventral foregut endoderm (FIG. 20). In the day 14 H+M condition. it was observed that heterogeneous CD31 expression, but not in the GF(−) condition (FIG. 2P), consistent with transient endothelial expression in hepatogenesis. Consistent with the early HB phenotype, it was observed that nuclear expression of FOXA2 and HNF4A in both the GF(−) and H+M conditions (FIG. 2Q). CDX2 expression was lower in the H+M condition compared to the GF(−) condition. Based on this, the H+M treated cells were an early hPSC-HB population. Long term culture in H+M medium was performed, which showed an AFP, ALB, and TBX3+cell population. This indicates that day 14 HBs were stable in hypoxic culture up to day 24 and presumably beyond (FIG. 8K).

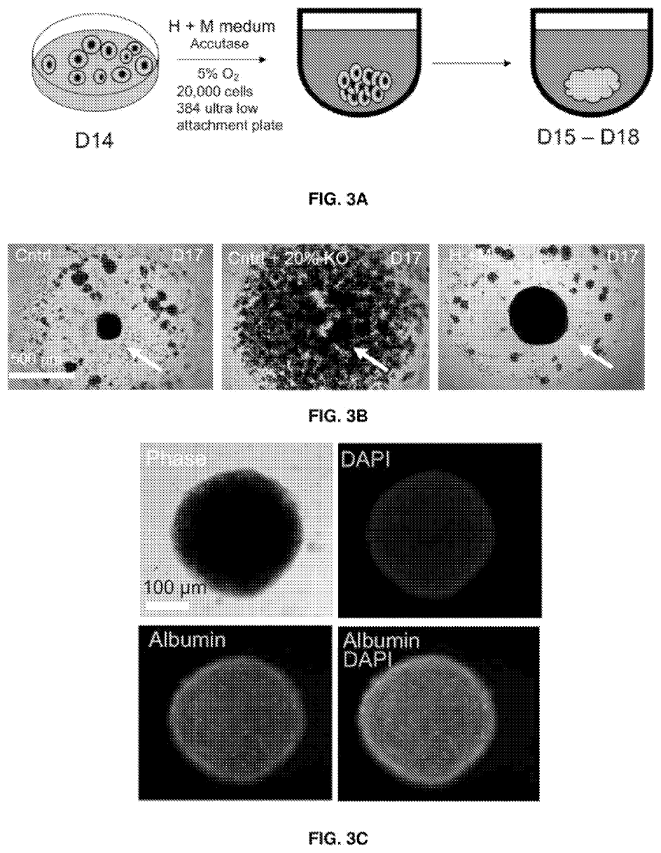

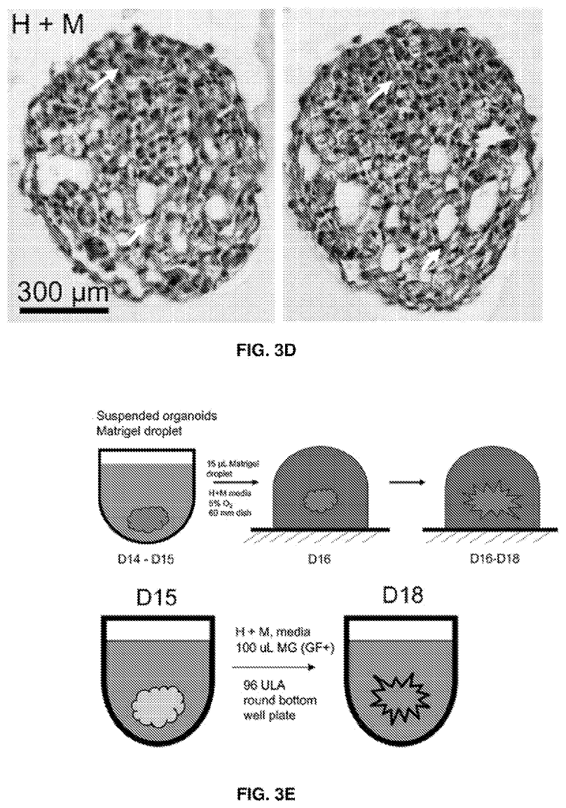

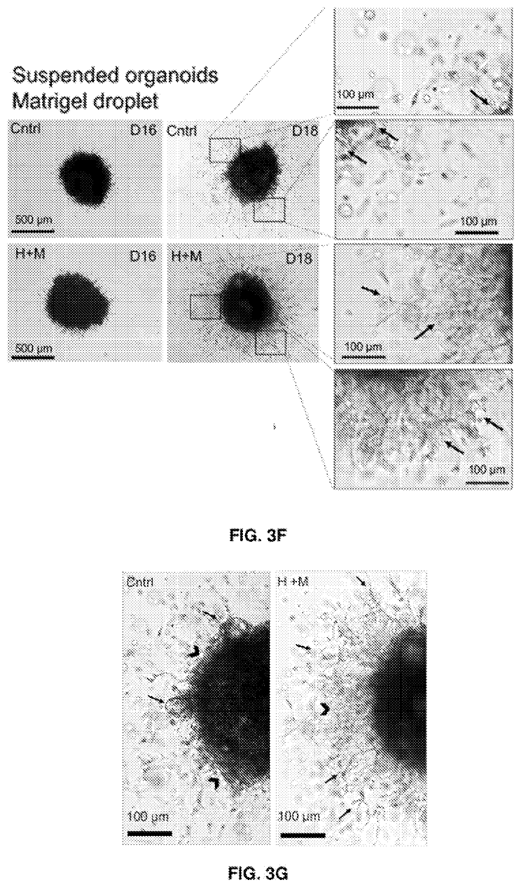

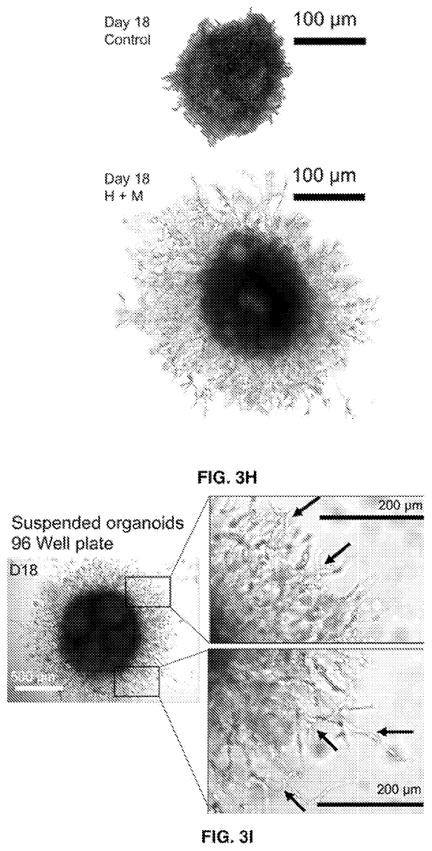

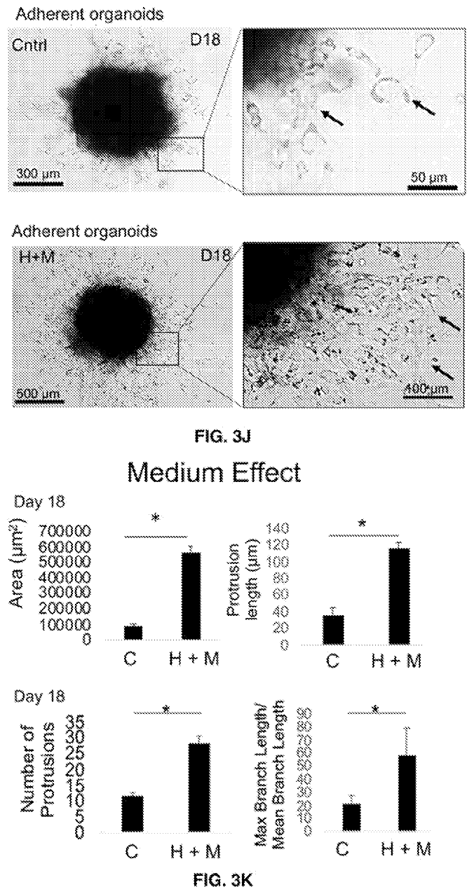

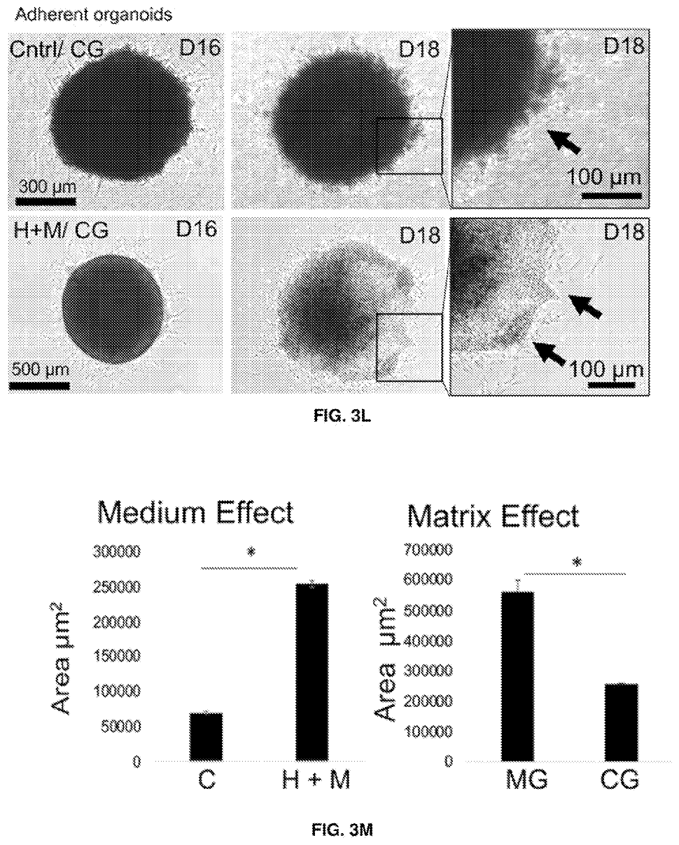

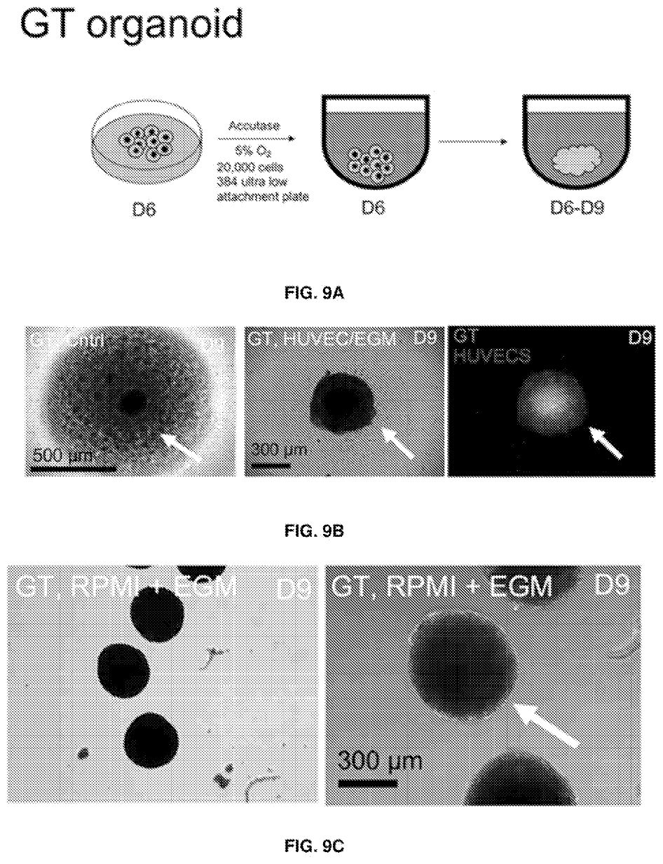

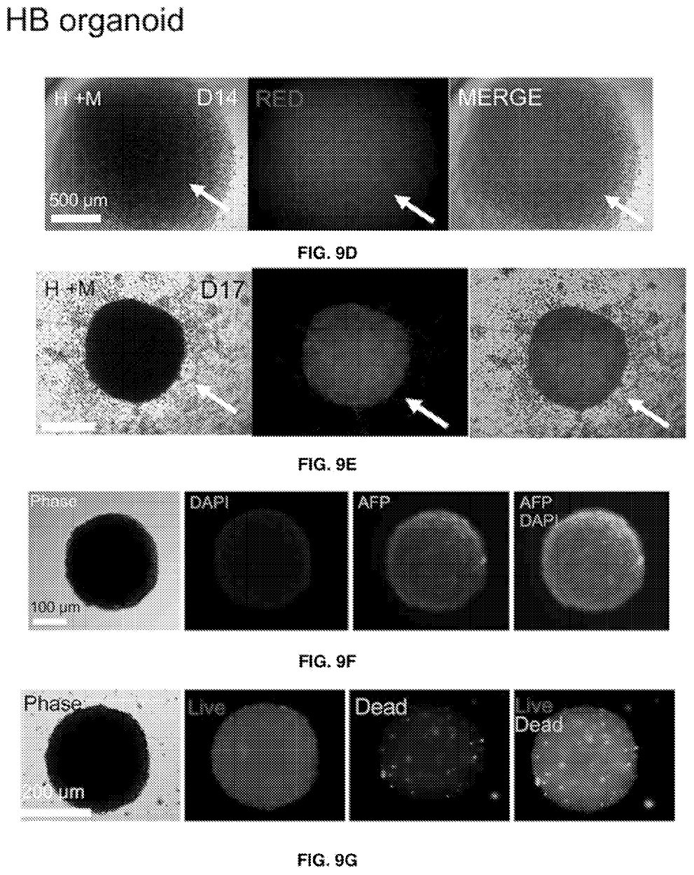

Effects of medium on growth and CCM from hPSC-HB organoids in extracellular matrix droplets. The next goal was to form compact organoids with the early hPSC-HBs. to recreate the hLD-MESC and trigger CCM. growth. and further early HB maturation. Experiments were performed to elucidate factors which cause organoid compaction, or condensation. It was found that both H+M medium, and MES-derived cells. promote compaction (FIG. 9A-G). Similarly. for hPSC-HBs in H+M medium (modified EGM formulation) compaction resulted (FIG. 3A-B), establishing that H+M favors organoid compaction. Immunostaining of organoids demonstrated evidence of both ALB expression (FIG. 3C), as well as other liver proteins (FIG. 9F) and hPSC-HB organoids were viable (FIG. 9G). Tissue sectioning demonstrated well-organized compact tissue with clusters of epithelial cells and several cystic regions (FIG. 3D). Early hPSC-HB organoids were then employed to model the LD-MESC by transferring them to MG droplets under hypoxic conditions (FIG. 3E, top). 96-well systems submerged with MG showed similar results (FIG. 3E, bottom). When day 15. H+M treated hPSC-HB organoids were transferred to MG droplets, control medium demonstrated minimal or no CCM by day 18 (FIG. 3F, top two panels, inset). whereas H+M medium resulted in radial finger-like migrating protrusions with evidence of branching and webbing (FIG. 3F, bottom two panels, inset). shown at higher magnification (FIG. 3G). It was observed that peripheral cystic structures in control medium-treated organoids (FIG. 3G, left), and in the H+M condition. migrating, branching cell strands that extend well over 100 μm (FIG. 3G, right). Filtering improved visualization of this phenotype (FIG. 3H). Similar migrating strands were also observed in a 96-well plate model (FIG. 3I). Since it was challenging to observe and analyze 3D CCM. adherent organoids were analyzed. which were plated slightly lower in the MG droplet. In adherent organoids. it was again observed that minimal CCM in the control, but extensive CCM in the H+M treated condition (FIG. 3J). The data indicates that CCM and growth were linked. as significantly more growth area were observed (outgrowths), as well as measures of CCM, in H+M treated adherent organoids compared to control organoids (FIG. 3K). Also evaluated were collagen gels droplets instead of MG, which known to be stiffer (2 mg/ml) (FIG. 3L). Sheet-like growth in the H+M condition (FIG. 3L, bottom panels and inset) were observed compared to control, which exhibited minimal growth (FIG. 3L, top panels and inset), with significant differences (FIG. 3M, left). Overall growth was significantly higher in H+M treated. MG organoids, compared to H+M treated, CG organoids (FIG. 3M, right). In summary, these data demonstrates that H+M medium stimulates growth and morphogenesis compared to control, and that MG induces collective branching, whereas CG induces sheet-like CCM. Moreover, these extracellular matrix droplet model models certain aspects of the LD-MESC.

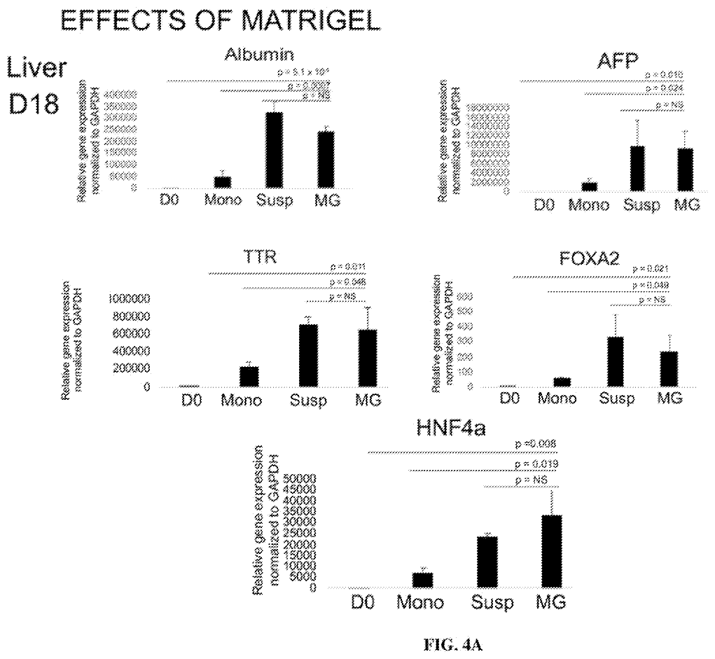

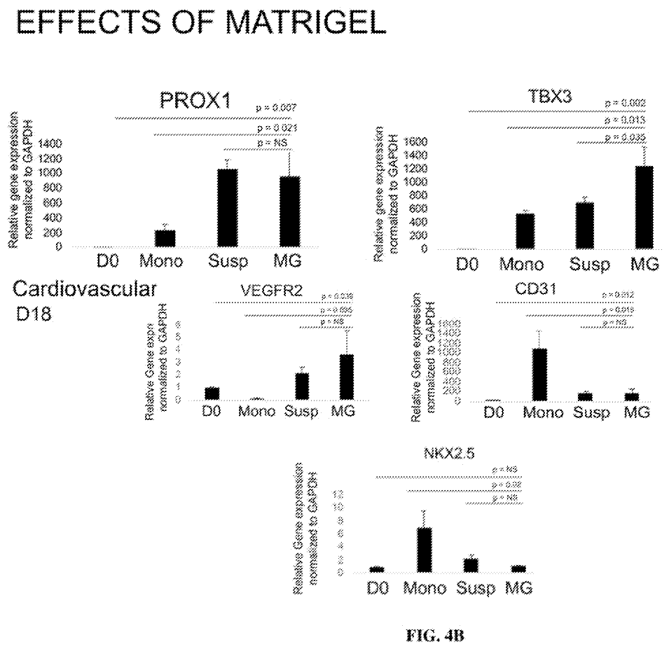

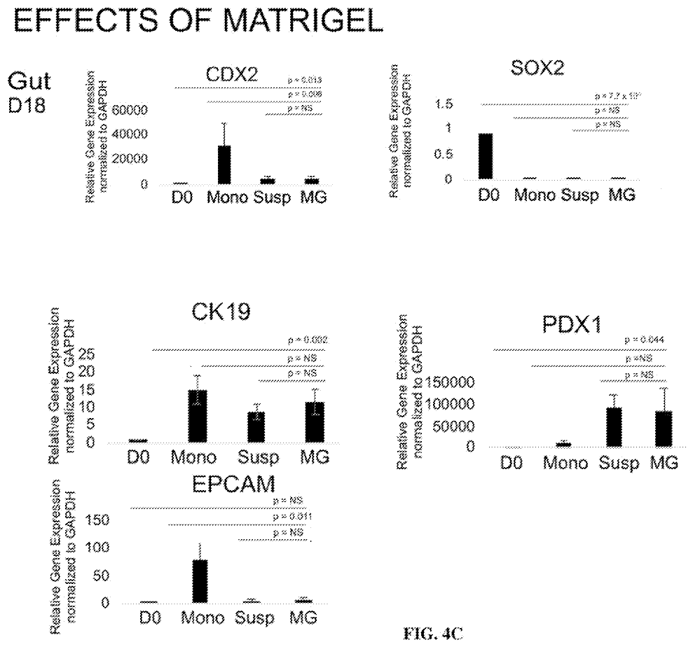

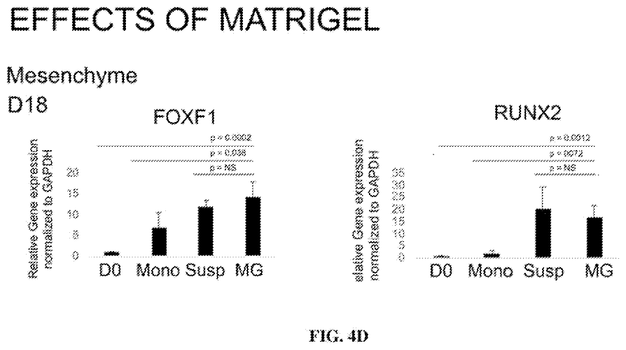

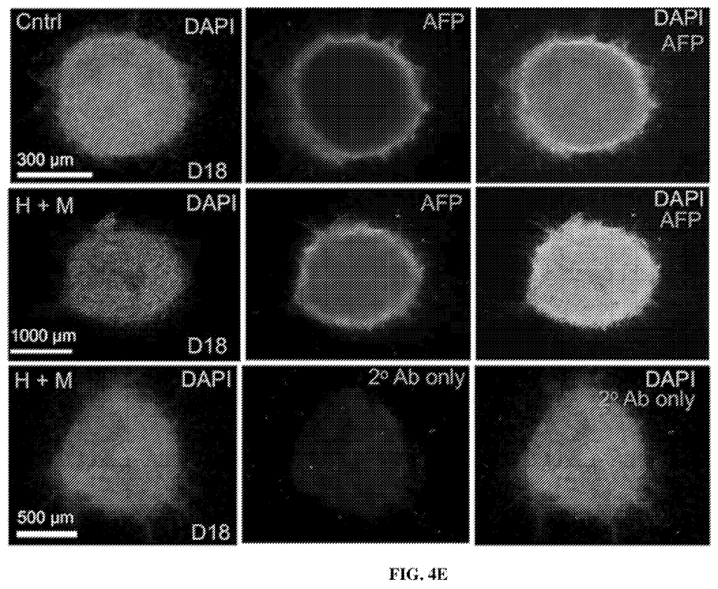

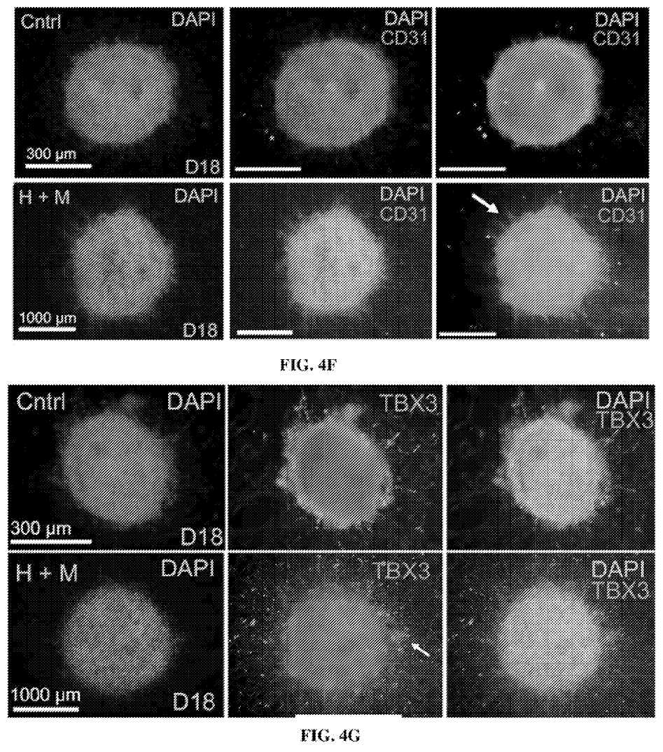

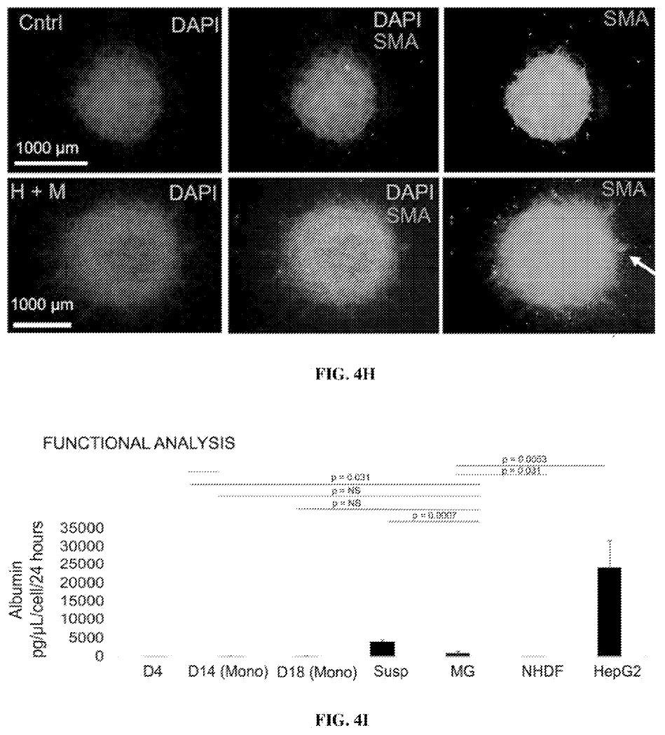

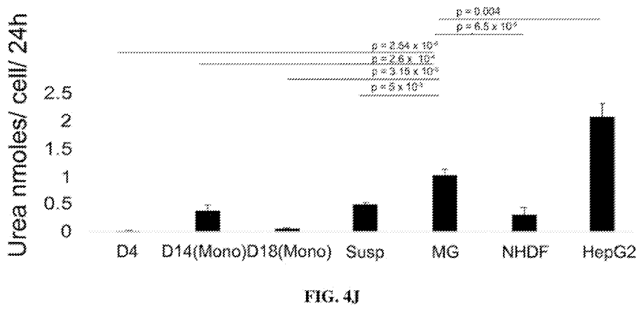

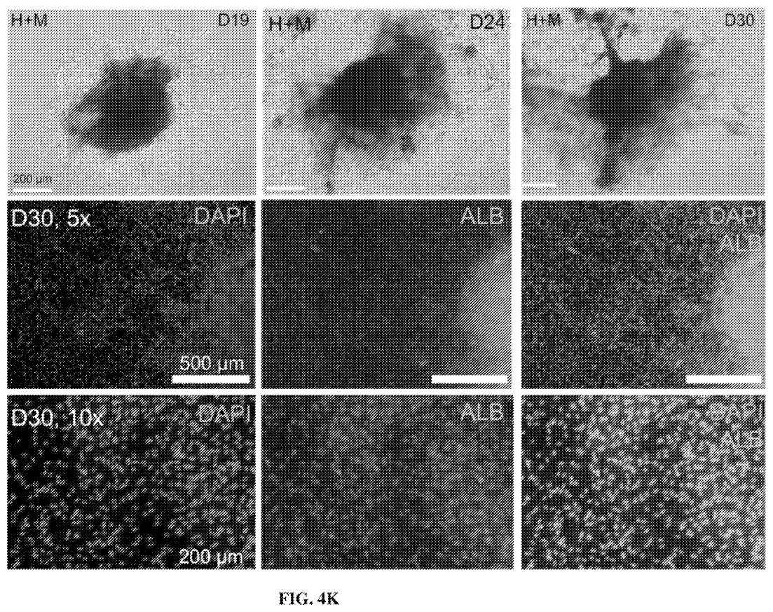

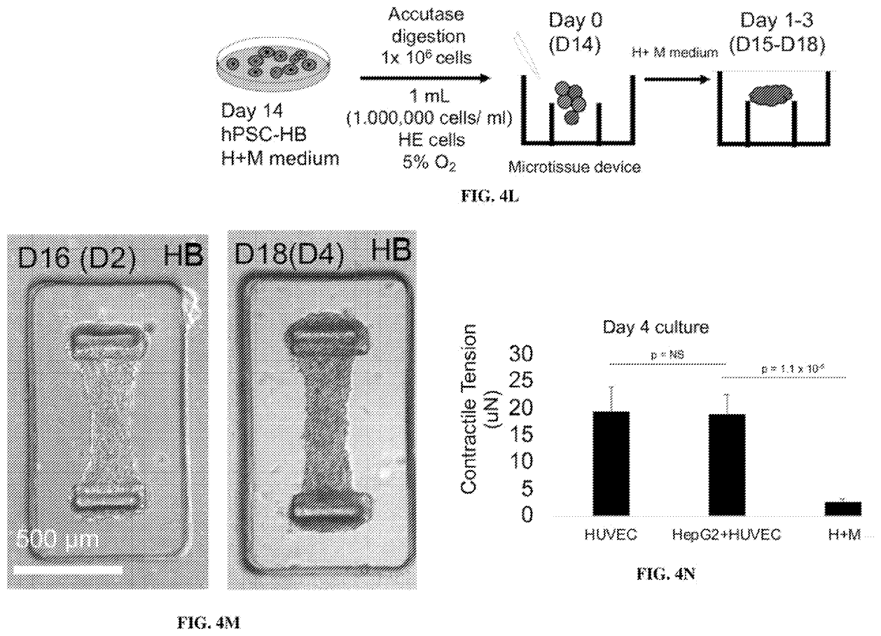

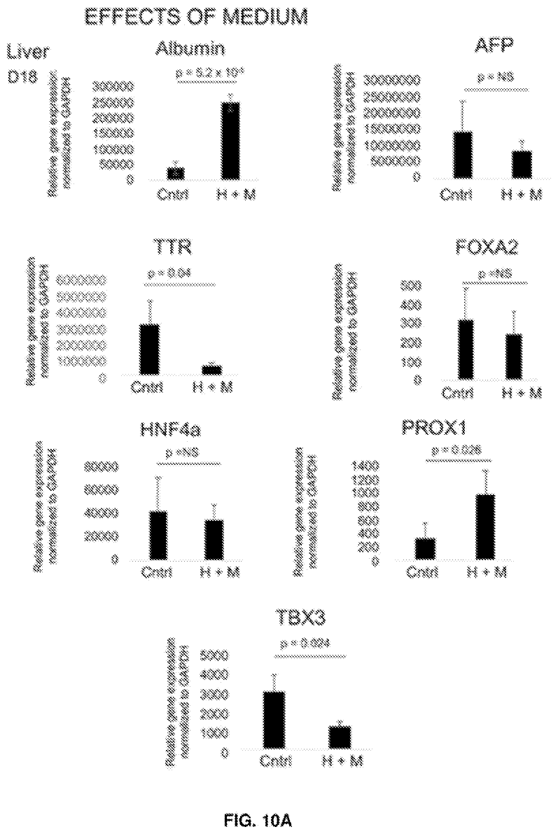

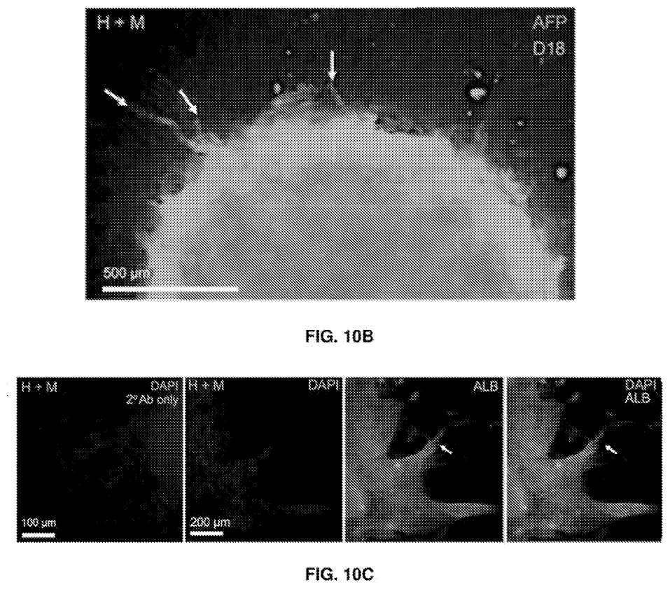

Day 18 LD-MESC organoids from extracellular matrix droplets express an immature hepatic signature in the absence of maturating factors. It was hypothesized that the hLD-MESC model links migration with further maturation, in the absence of additional instructive/maturating factors. Gene expression analysis demonstrated that H+M medium resulted in significantly higher ALB, PROX1, and significantly lower TTR and TBX3 expression (FIG. 10A). In the H+M condition, it was determined how culture configuration (monolayer (MONO), compared to suspension (SUSP), and MG (MG and CCM) effects gene expression (FIG. 4A). Hepatic gene expression, in all cases, was significantly higher in both SUSP and MG compared to MONO. Cardiovascular gene expression was significantly higher for CD31 and NK2.5 in MONO compared to both SUSP and MG, but significantly lower for VEGFR2 for MONO compared to both SUSP and MG. Mesenchyme markers FOXF1 and RUNX2 were significantly lower in MONO compared to both SUSP and MG conditions. The gut markers SOX2, CK19, and PDX1 were unchanged between MONO and both SUSP and MG conditions, while CDX2 and EPCAM were both significantly upregulated in MONO compared to SUSP and MG conditions. Thus, in MONO culture, there is an increase in cardiovascular and gut gene expression, and decreased hepatic expression, as compared to SUSP and MG. When comparing SUSP and MG conditions, hepatic, cardiovascular and mesenchyme, and gut were equivalent except for TBX3, which was significantly higher in MG condition. Overall, the data suggests that compaction in H+M medium enhances differentiation, and that the MG condition, which exhibits CCM, maintains hepatic differentiation markers and upregulates TBX3 expression. Further, there are extensive increases in hepatic gene expression in SUSP and MG conditions compared to the MONO condition.

hLD-MESC model demonstrates liver protein expression and function. It was hypothesized that the hLD-MESC model, which exhibits CCM. also co-expresses liver and mesodermal protein expression. This was based upon extensive mesoderm emergence from the LD. as well as the potential presence of HM cells. Organoid immunostaining showed AFP was expressed in both the control and the H+M condition (FIG. 4B top and middle panel). It was noted that the bright center will saturate the image with traditional thresholding and the intensity of the image had to be increased to visualize migrating strands at the edge. Using this approach. it was found that migrating strands were indeed AFP positive (FIG. 4B, middle panel), CD31 low (FIG. 4C top, bottom panel), TBX3 low (FIG. 4D top, bottom panel). and SMA high (FIG. 4E, top, bottom panel). ELISA was performed for ALB secretion (FIG. 4F). MONO condition showed low ALB secretion, ALB secretion was higher in SUSP vs. MG condition and the MG and SUSP condition were significantly lower than HepG2 cell secretion (FIG. 4F). Urea secretion was then analyzed. Urea secretion in MONO culture showed an increase but then a significant decrease from ay 14-day 18. (FIG. 4G). The MG condition secreted significantly more urea than the SUSP culture and NHDF condition and was significantly lower than HepG2 (FIG. 4G). Thus, the MG condition demonstrated lower ALB secretion. but higher urea secretion when compared to the SUSP condition. Long term culture was performed of day 18 hPSC-HBs in MG droplets until day 30 (FIG. 4H). Morphological analysis shows progressive rapid and irregular growth accompanied by CCM (FIG. 4H). Immunoanalysis demonstrates stable ALB expression (FIG. 4H. middle and bottom rows). This suggests the cells are stable and robust in long culture. Overall, these data suggest that the migrating hPSC-HBs display an AFP+, ALB+, and SMA+ population. suggesting a partial mesenchymal phenotype, and secreted higher urea than SUSP organoids. and demonstrating robust culture through at least Day 30.

Early hPSC-HBs exhibit a functional mesenchymal phenotype in a functional assay with bioengineered tissue culture platform. Numerous studies demonstrate a hepato-mesenchymal (HM) hybrid phenotype arises during early development. and mouse fetal liver and these cells could provide leader cells for CCM and potentially provide a niche for hematopoietic stem cells in the fetal liver. The hypothesis was tested of whether day 18 H+ M treated cells exhibited a functional, mesenchymal, HM phenotype. An established bioengineered device was used that evaluates tissue tension in mesenchymal-derived microtissues. The device is a microfabricated pillar culture system predicated upon supporting formation of a microtissue with mesenchymal properties. A series of preliminary experiments with cell lines were performed to establish the requirement of mesenchyme for forming a hepatic microtissue and measuring tissue tension (Sup FIGS. 6A-F). Day 18 hPSC-HBs robustly formed microtissue in the microfabricated pillar culture system, indicating a mesenchymal phenotype (FIG. 4I-J). Contractile tension analysis was performed, and it was demonstrated that the hPSC-HB microtissue generated tension, but at significantly lower levels than the HUVEC and HUVEC-HepG2 systems (FIG. 4K). Thus, these data support the premise that day 18 HBs bear a HM phenotype.

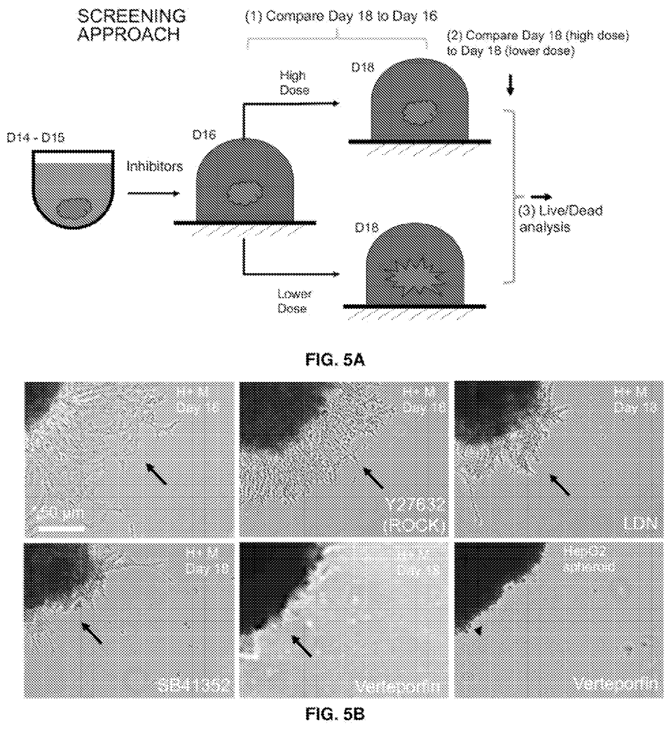

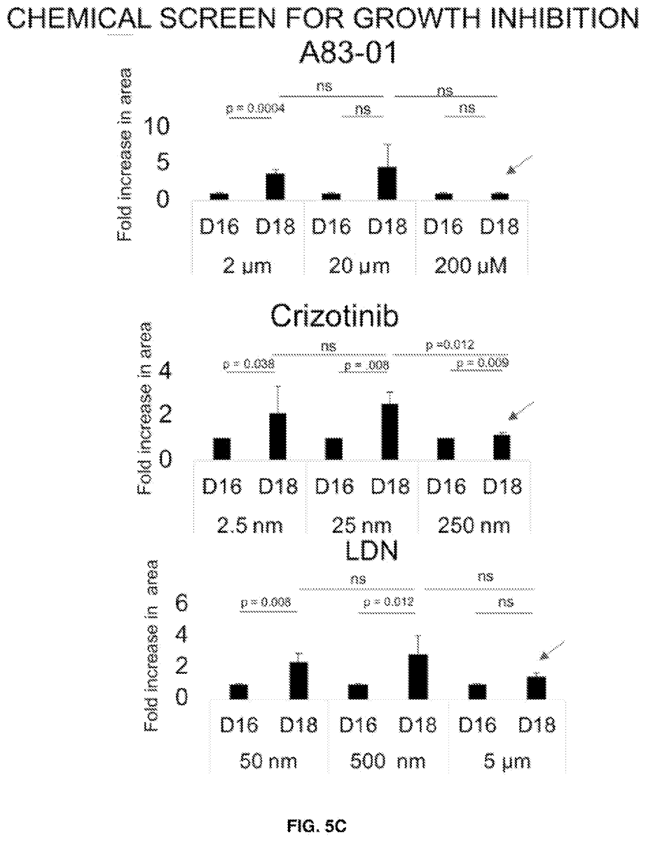

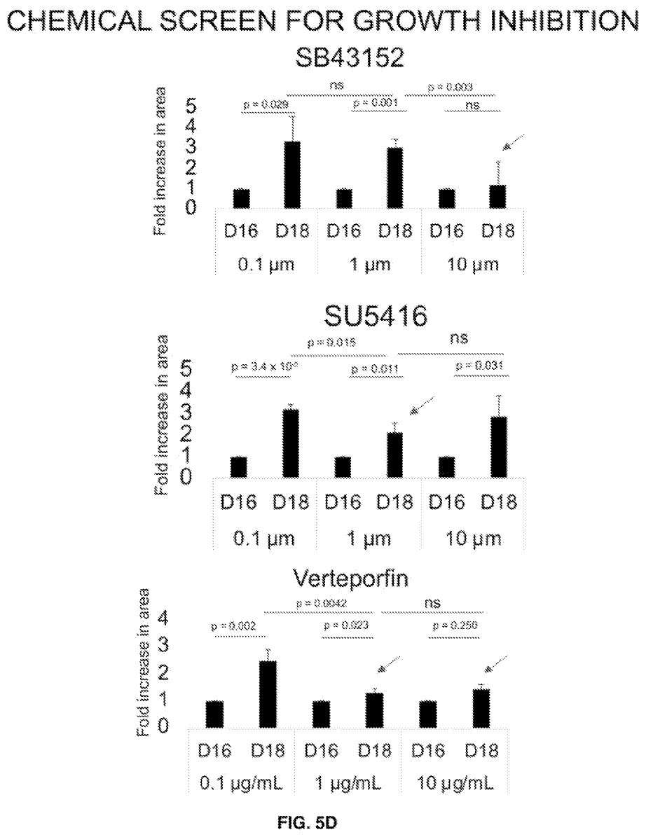

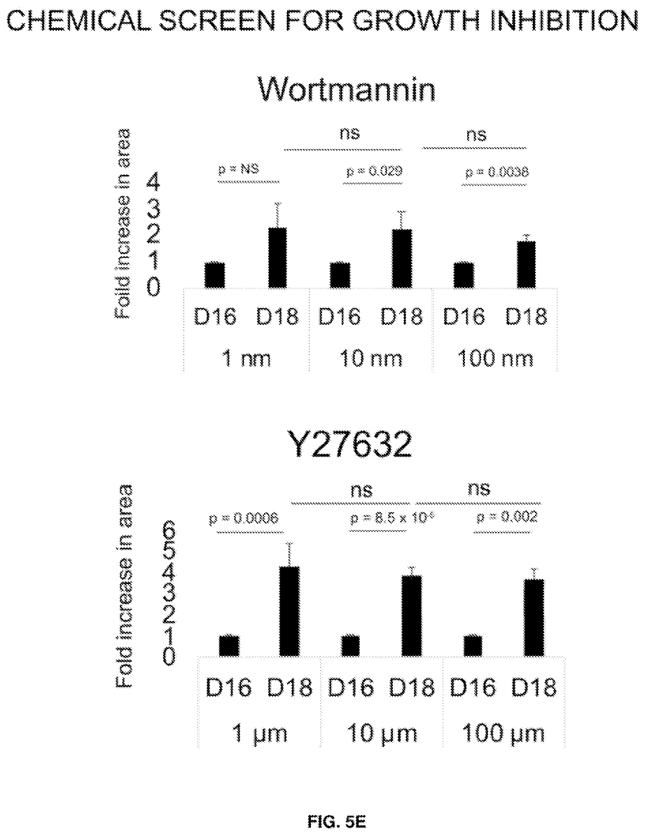

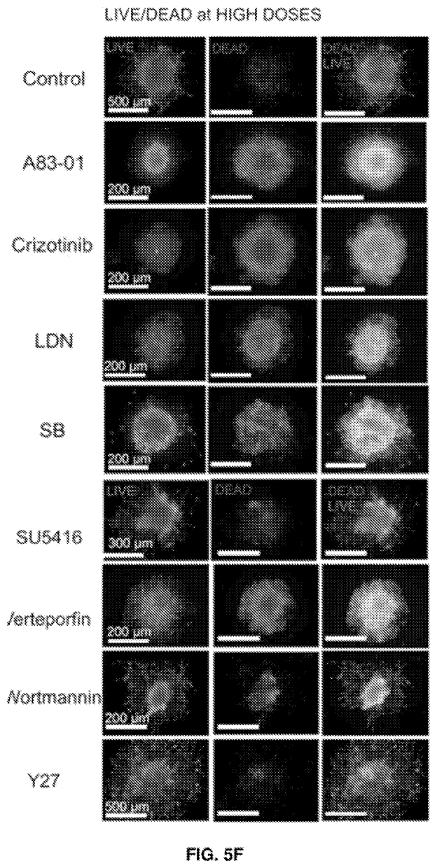

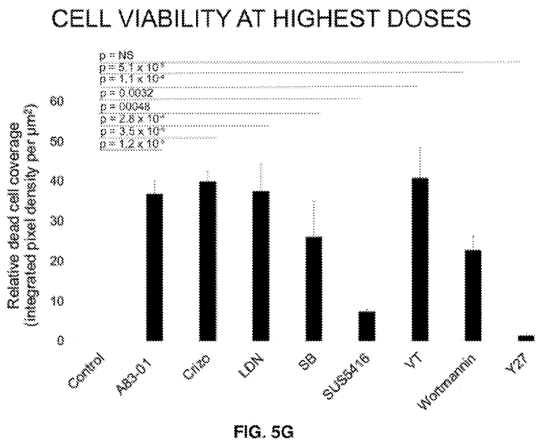

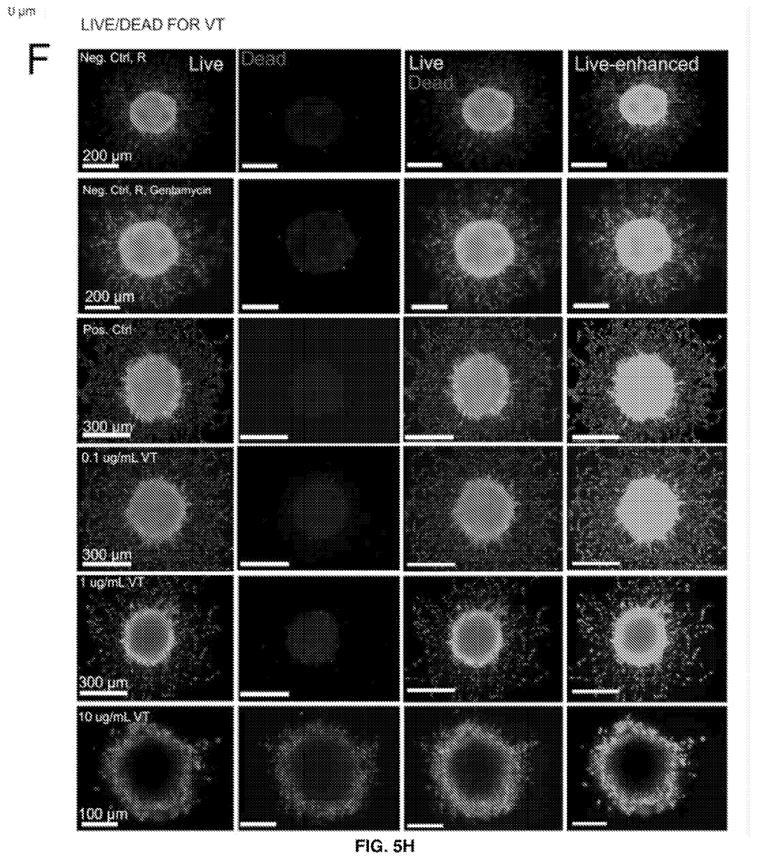

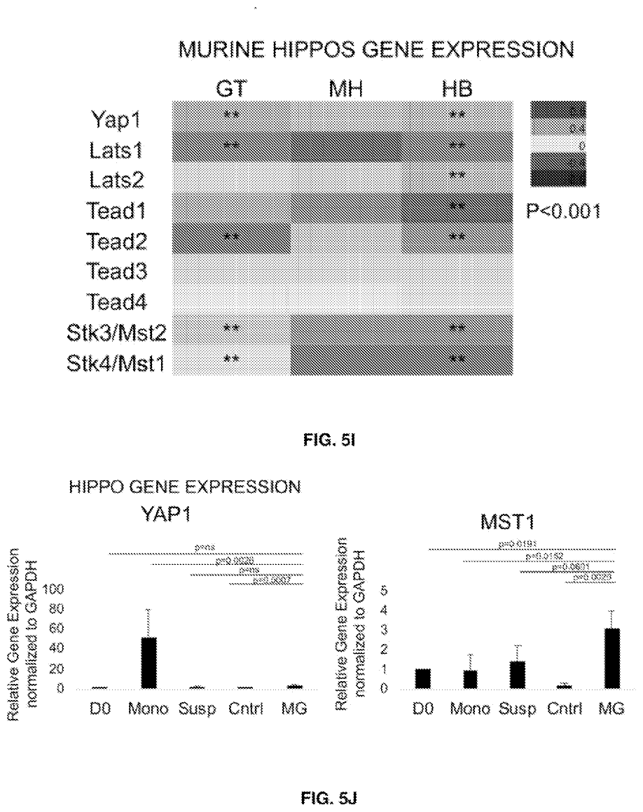

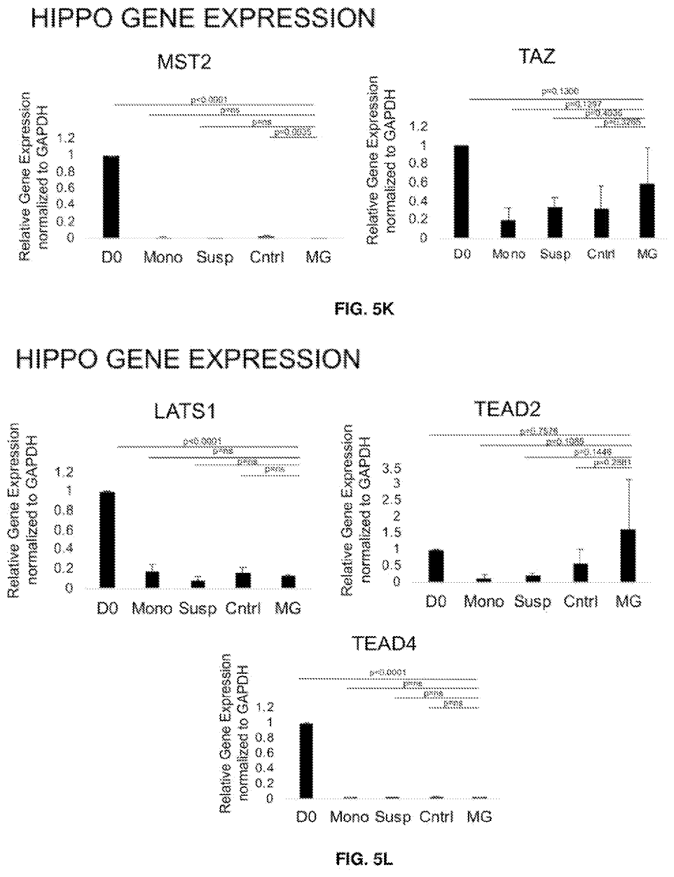

Screening of pathway inhibitors demonstrates that Hippo pathway controls triggering of LO. The bioinformatic analysis demonstrated that E9.5 MH is associated with an up-regulation of numerous signaling pathways, which are immediately downregulated (E10.5) (FIG. 1K, 1O, 1U). 35 potential candidate signaling pathways were identified in eLO, and pathway activity was tested in the day 18 hLD-MESC adherent organoid model by performing an in vitro chemical screen with 3 criteria (Methods and FIG. 5A). After the addition of chemical inhibitors (control, Y27632, LDN, SB41352, VT) at the highest doses, it was observed that significant reduction in growth/CCM in response to the VT treatment (FIG. 5B). The screen was then expanded to twenty-four inhibitor conditions (based on FIG. 1U), with eight candidates (three concentrations per candidate). Dose responses up to three orders of magnitude were performed. Inhibition of CCM/growth for A83-01 (high), Cristozinib (high), LDN (high), SB43152 (high), SU5416 (intermediate dose), Verteporfin (VT) (high, intermediate), were identified, but not for Wortmannin, or Y27632 (FIG. 5C, arrows). Interestingly, at intermediate doses, the only inhibitors that significantly reduced CCM were VT and SU5416. It was hypothesized that the lack of CCM/growth at the highest concentrations could be due loss of cell viability, rather than just blocking CCM/growth. Indeed, it was found that all inhibitors except SU5416 and Y27632, caused cell death at highest doses, and were removed (FIG. 5D-E). The two remaining inhibition conditions at intermediate doses were SU5416 and VT, which we found decreased CCM/growth but did not increase cell death (FIG. 5F), and thus were positive hits of the screen. Given the success of VT in the screen, and the global analysis of scRNA-seq data demonstrating Hippo pathway activation (FIG. 1R), it was concluded that the Hippo pathway (YAP-TEAD signaling) plays a key role in linking growth and CCM. To support this, murine scRNA-seq data for mediators of Hippo were re-analyzed, and it was found that they were all upregulated in the MH population (FIG. 5G). Next, analysis of gene expression of Hippo mediators in the day 18 hLD-MESC system showed that in the MG condition, YAP1 and MST1 was significantly upregulated in the MG condition compared to control, MST2 was significantly downregulated to control, with no differences in TAZ, LATS1, TEAD2, TEAD4 (FIG. 5H). In summary, the chemical screen recovered VT as a positive hit. Analysis of Hippo mediators in the MH population and in the hLD-MSC model suggest growth/CCM is linked to changes in Hippo pathway.

LO is a central, cross disciplinary topic in regenerative medicine, and hPSC are a valuable tool for interrogating LO, bringing together basic and practical applications. Progress in the field of LO has been comprised of significant genetic studies, and both single cell RNA-seq analysis and hPSC-HEP studies, but

eLO has yet to be scrutinized. In fact, current hPSC approaches do not account for eLO. Based on the premise that the E8.5-10.5 stage LD-MESC is significant for linking signaling with CCM, growth, and differentiation, bioinformatics analysis was employed, and both in vivo hPSC and in vitro hPSC investigation, to not only elucidate the transcriptome of the E9.5 MH cells, but also to develop more accurate models of these developmental stages. Several novel findings push forward the field of LO, including novel 3-step protocol and medium formulation, a new cell population that has not previously has been induced or isolated, a simple but effective in vivo model, extensive knowledge and analysis of the E9.5 niche and transcriptome, and a molecular mechanism involved in hPSC liver organoid growth/CCM. In terms of accuracy of modeling the LO, this in vivo model transplant model demonstrates exponential growth at approximately the rate of in vivo liver growth and our in vitro model demonstrates rapid ALB activation, collective cell migration, and growth, function of immature HBs, and establishment of the hepatic nuclear GRN (FOXA1, FOXA2, FOXA3, HNF1α, HNF1β, HNF4α, HNF6, HEX, TBX3, and PROX1). The 3-step culture platform in this example could be used as an initial step in hPSC-HEP differentiation, and this study may serve as a resource for the LD-MESC. The clinical implications of this example may be for understanding and treating migrating or metastasizing hepatocellular carcinoma and for evaluating the therapeutic role for both LD-MESC concept and MHs in human liver repopulation.

The bioinformatics analysis of the eLO process provides a full resource of the LD-MESC and how it triggers eLO via MH cells, which opens up many potential areas of investigation. Based upon the data here, the description of the events of eLO can be extended to comprise not only increased signaling, CCM, exponential growth, and rapid differentiation, but also metabolic programming, emergence of MES-derivatives, and the role for nascent liver immune system in growth. These integrated transformations that arise provide numerous research directions for future investigation in this crucial area. While functional analysis is still required, these data indicates that the MH cells, arising from the LD-MESC, have a unique transcriptomic signature, with elevated signaling, immune pathways, and stress responses. Interestingly, it was found that upregulated signaling pathways in MH are also predominantly upregulated during murine liver regeneration. Overlapping pathways between MHs and regenerating hepatocytes include C-met (HGF receptor), EGFR, FGFR, Wnt, TGF-β, VEGFR, Hippo, Notch, IGF-1, NIK/NF-kB, p21, p53, TNF, IL-6, and endocannabinoids. Moreover, the upregulated list of signaling pathways in MH is the surprising balance in number between traditional soluble signaling pathways and immune signaling pathways. The REACTOME (pie chart) analysis demonstrated an increase in up-regulation in immune system signaling, and the ENRICHR pathways confirmed the list of potential immune signaling pathways, including TNF, IFN-γ, Oncostatin, Interleukins (IL-1, IL-2, IL-4, IL-5, IL-6, IL-11, IL-12, IL-18) as well as B and T cell-receptor signaling, NIK/NF-kB, and Calcineurin-NFAT signaling. Finally, the upregulated pathways include an extreme pattern of the global cell stress response, a pattern that included HIF1α (oxygen), AMPK (energy), mTOR (nutrient), FoxO (oxidative), DNA damage-related stress, with evidence of endoplasmic reticulum (ER) stress. Additionally, it was observed the activation of PI3K-Akt, suggesting PI3-AKT-mTOR axis is active in the MH cells, which is active also in cancers. This unique transcriptome suggests that the eLO deserves further attention in hPSC protocols and can be used to model cancer.

Notably, in the E9.5 MH population, not only was Hippo signaling upregulated in the E9.5 MH population, it was identified independently in our novel hPSC protocol and chemical screen for mechanisms of CCM and growth. Hippo pathway integrates mechanical forces (integrins and Rho signaling), intercellular adhesion, WNT signaling, and stresses (osmotic, oxygen, energy) to alter YAP/TAZ signaling, leading to CCM and growth. Consistent with Hippo signaling and increased CCM, lung branching morphogenesis genes were also upregulated, which has not previously been reported (FIG. 1S). Further, the in-depth analysis of Hippo mediators with heatmaps in the 9.5 MH population, and in the hLD-MESC, as well as the results of our chemical screen for signaling pathways in hLD-MSC, all pointed towards a major role for Hippo (YAP-Tead) in not only mediating migration, but also linking migration with growth and differentiation. These data collectively implicate Hippo in integrating not only signaling pathways with migration/growth/differentiation, but also metabolic programming, stress pathways, biomechanical niche, and chromatin modification/epigenetics. Further studies with this model can be employed to discover potential mechanisms by which this integration may occur.

In summary, the hLD-MESC model in this example, is a culture system which links migration, growth, and differentiation, can be used for in vivo tissue growth, and exhibits signaling pathways not seen in monolayer culture. This novel LD-MESC model can serve as a platform further investigation into early LO. Further, enhanced imaging analysis of organoids, with techniques like spatial transcriptomics and knockout and functional studies of VEGFR and EGFR, will help determine mechanisms of CCM and the phenotype of the leader cells.

FIG. 1 shows bioinformatics analysis of murine scRNA-seq data during early LO demonstrates coordinated transcriptomic changes during CCM. FIG. 1A 3D images of the E9.0 Liver Diverticulum (LD) (right). LD is shown to be surrounded by mesodermal-derivatives (MES). FIG. 1B Description of early murine LO between E8.5-13.5. Double plot shows correlation from this analysis scRNA-seq and Northern blot. FIG. 1C Hepatic lineage map used in this study. FIG. 1D Heatmap filtered for FDR<1×10⁻²⁰ and sorted by log2-fold-change. FIG. 1E A force-directed layout plot (n=2332 cells), from clustered based on re-grouping. FIG. 1F Force-directed layout plot analysis of liver differentiation markers (Alpha-fetoprotein (AFP), Albumin (ALB)). FIGS. 1G-1J Pie chart created with REACTOME containing gene categories for DEG lists (log2fc>0.5,FDR<0.05). Total DEG and the number of gene occurrences shown. FIGS. 1K-1L Upregulated pathways (GO BP, Kegg, DAVID) for MH vs. GT and HB. For metabolic pathways, for MH vs. DE and GT was used. FIGS. 1M-1N Same as above except downregulated (log2fc<−0.5, FDR<0.05) gene list. FIGS. 1O-1P Ranked, alphabetically sorted, upregulated pathways (GO BP, Kegg) for both signaling (FIG. 1O) and metabolism (FIG. 1P). FIGS. 1Q-1R KEGG oxidative phosphorylation pathway (FIG. 1Q) and GO BP hippo signaling pathway (FIG. 1R) Heatmaps. FIGS. 1S Pathway heatmap analysis, with averaged value, for select pathways for GT, MH, and HB cells ** is p<0.001. FIG. 1T Heatmap for FOXA factors. Analysis same as in FIG. 1S.

FIG. 2 shows in vivo transplantation protocol and in vitro protocol for modeling early LO. FIG. 2A Schematic of 4-week, hLD-MESC transplant (NOD-SCID) model, with hPSC-DE:HFF (4:1 ratio) plus GF-free MG subcutaneously transplanted. FIG. 2B Gross images of teratoma, hPSC-DE:HFF, HEPG2 tumor, 4 weeks post-transplantation. FIGS. 2C-E Histological analysis (Hematoxylin and Eosin) of Teratoma (neuro-tubular structures (arrow), connective tissue (arrow)); hPSC-DE:HFF (DE-derived (blue) (arow), HFF (orange)), last image segmented; HEPG2 tumor. FIG. 2F Bar graph of qRT-PCR gene expression in vivo human liver differentiation. AFP/ALB (n=3), DE:F (n=5), HEPG2 (n=4), mean±SD. FIG. 2G Three protocols for hPSC-HB induction (5% O₂); Growth factor (GF(+)) protocol; GF(−) protocol with serum-free SFD medium, H+M protocol EGM-2 modified medium. FIG. 2H Overall schematic summarizing 3 stages for the H+M differentiation protocol. Early hepatoblast (HB) stage-day 4-14 in monolayer; Compaction stage—day 14-15; LD-MESC stage-organoid in MG droplet. FIG. 2I Same as FIG. 2H, except morphological hallmarks are shown. FIG. 2J Morphological analysis during HB differentiation. Endoderm (END)—cuboidal; GF(+): elongated; GF(−): cuboidal; epithelial (E) and non-epithelial (NE) elements (arrows); H+M condition: cuboidal, and NE elements. FIG. 2K Gene expression analysis (qRT-PCR) of the effects of medium on hepatic differentiation; GF(+) (n=3), GF(−) (n=3), and H+M (n=4); mean±SD. FIG. 2L Same as FIG. 2K except effects of time: day 0 (n=3), day 4 (n=3), and day 14 (n=3); mean +SD. FIG. 2M Enzyme-linked immunoabsorbance assay (ELISA) analysis for ALB secretion; day 8 (n=4), day 11 (n=5), day 14 (n=3). mean±SD. FIG. 2N Immunocytochemistry of day 14 hPSC-derived HBs in H+M and GF(−) conditions. DAPI (UV filter), FITC, and merged (UV and FITC) are shown. HBs are stained for AFP (above) and ALB (below). FIG. 2O Same methods as in FIG. 2H except hPSC-derived GT endoderm (day 6 cells) were stained by immunocytochemistry, and ALB (liver), CDX2 (hindgut), and SOX2 (foregut) were targeted. FIG. 2P Same methods as in FIG. 2H except CD31 (vascular differentiation) was targeted. FIG. 2Q Same methods as in FIG. 2H except hepatic TFs FOXA2, HN4A, and gut TFs were assessed targeted, as was the intestinal marker CDX2.

FIG. 3 shows induction of CCM from hPSC-derived HB organoids FIG. 3A Schematic of day 14 hPSC-HB organoid formation FIG. 3B Day 17 images hPSC-HB organoid compaction. Left: control RPMI basal; Middle: RPMI basal medium+20% KOSR; Right: H+M; Right: Bar graph quantitation: Arca (n=4), perimeter (n=4); mean±SD. FIG. 3C Immunofluorescence staining of ALB (middle row), on day 17 hPSC-HB whole organoids. FIG. 3D H+E images of day 17 hPSC-HB organoids; above arrows-uniform epithelium; below arrows-non-uniform-cystic like structure. FIG. 3E Schematic of day hPSC-HB organoid suspended in Matrigel (MG) droplet culture (60 mm dish) or 96-well. FIG. 3F Phase contrast images of day 18 migrating hPSC-HBs treated in control and H+M medium. FIG. 3G Same as FIG. 3F except larger; Control: cyst like structures (arrow), minimal CCM (arrowhead). H+M organoids (right) demonstrate CCM. FIG. 3H Same as FIG. 3F except filtered images to remove out cells that were out of focus. FIG. 3I Phase contrast images of H+M treated hPSC-HB organoids, on day 18 in 96-well plate format. Extensive radial CCM is demonstrated. FIG. 3J Phase contrast images of control (arrow: minimal CCM) and H+M treated (arrow: CCM) hPSC-HB an adherent organoid model. FIG. 3K Bar graphs analysis of images in FIGS. 3I-J. Arca (P=3×10⁻⁺), protrusion length (P=2.8×10⁻⁴ ), number of protrusions (P=1.4×10⁻³), and the max/mean branch length ratio (P=1.4×10⁻²), mean±SD. FIG. 3L Same as FIG. 3J except collagen gel (CG); c images of control (arrow: minimal CCM) and H+M treated (arrow: CCM). FIG. 3M Bar graphs analysis in adherent CG droplets. Left: Effects of medium for CG; Right-Effects of MG vs CG for H+M treated.

FIG. 4 shows gene and protein expression of control and H+M organoids cultured in MG droplets. FIG. 4A Gene expression analysis comparing day 18 monolayer (Mono), day 18 suspended organoids (Susp), and day 18 MG droplet (MG) conditions in H+M medium conditions. Day 0 (n=3), Mono (n=3), Susp (n=3), and MG (n=3). p-values shown; mean±SD. FIGS. 4B-E Immunocytochemistry of Control (top) and H+M treated (Middle and lower) day 18 whole organoids for AFP (FIG. 4B), CD31 (FIG. 4C), TBX3 (FIG. 4D), SMA (FIG. 4E); counterstained with DAPI and FITC. FIG. 4F ELISA analysis ALB secretion of day 4 (n=3), day 14 MONO) (n=3), day 18 MONO (n=3), day 18 (SUSP), day 18 organoids (n=3), NHDF cells (n=3), HepG2 liver hepatoblastoma (n=3); p-values listed; mean±SD. FIG. 4G Same as FIG. 4F, except urea secretion analysis. Conditions measured were same as in I. p-values listed; mean±SD. FIG. 4K Long term MG droplet culture organoids; Top: day 19, 24, 30 images; Middle: ALB Immunofluorescence; Lower-Same as previous except at higher magnification. FIG. 4L Schematic demonstrating culture of hPSC-HB in microdevices; day 14 hPSC-HB are harvested and replated in device in H+M medium. FIG. 4M Phase contrast image of microtissues in microdevices that form on day 2 (day 16, left) and thicken by day 4 (day 18, right). FIG. 4N Graph of contractile tension in microtissue; hPSC-HB (n=6), HUVEC/HepG2 (n=3); p-values shown; mean±SD.

FIG. 5 shows functional screen of small molecule signaling pathway inhibitors for inhibition of CCM in hPSC-HB organoids in MG droplet culture. FIG. 5A Schematic of functional chemical screen of signaling pathways that effect organoid growth/migration. FIG. 5B Images of treated organoids. Top-untreated control, ROCK treated, LDN treated; Bottom-SB41352 treated, Verteporfin (VT) treated, and HepG2 spheroids+V treated; Arrows show inhibition. FIG. 5C Data for each inhibitor screened; Fold-increase in arca (growth) with 3 concentrations per chemical inhibitor; Red arrows indicate positive hits; P-values listed; n=3 for each condition. Plotted is mean±SD. FIG. 5D Images of live/dead assay for cell viability after chemical treatment. FIG. 5E Quantitation of experiments in D); P-values shown; N=3 per condition; mean±SD. FIG. 5F Same as D but focused on VT treatment; enhanced images (green) shown. FIG. 5G Heatmap analysis of averaged hippo pathway mediator gene expression from mouse scRNA-seq data **p<0.01. FIG. 5H Hippo gene expression analysis in day 18 organoids; Monolayer (MONO), Suspension (SUSP), Control (CNTRL), MG (Migrating). One-way ANOVA using Tukey's multiple comparison test; mean±SD.

### Abbreviations

AFP alpha-fetoprotein
ALB albumin
BMP4 Bone Morphogenetic Protein
BSA Bovine serum albumin
CCM Collective cell migration
CHIR Wnt pathway agonist
DMEM Dulbecco's Modified Eagle's Medium
EDTA Ethylenediaminetetraacetic acid
EGF Epidermal growth factor
EGM-2 Endothelial growth medium 2
EHT epithelial to hepatic transition
FBS fetal bovine serum
FGF2 Fibroblast growth factor-2
iPSC Induced pluripotent stem cells
HEP Hepatocyte
H+M hepatic and mesenchymal
hESC human embryonic stem cells
hPSC human pluripotent stem cells
HBs hepatoblasts
HSCs hematopoietic stem cells
HE hepatic endoderm
HFF human foreskin fibroblasts
HGF hepatocyte growth factors
HPSC Human pluripotent stem cells
IMDM Iscove's modified Dulbecco's medium
LD liver diverticulum
MES Mesoderm
MG Matrigel (growth-factor free)
PBST Phosphate buffered saline tween 20
RT-PCR Real-time polymerase chain reaction
R3 IGF-1 R3-Insulin growth factor-1
SFD Serum free-differentiation
STM Septum transversum mesenchyme
TFs Transcription factors
VEGF Vascular endothelial growth factor

### EXAMPLE 3

This example illustrates various materials and methods used in the previous examples. Bioinformatics Analysis used scRNA-seq analysis of published eLO data. Further details below. In vivo transplantation assay modeled the hLD-MESC in vivo. Further details below. Design of culture system modeled the hLD-MESC in vivo. Further details below

Bioinformatics Analysis. Data Collection, Normalization and Filtering. Normalized data files from the Lotto et al. (Lotto, Drissler et al. 2020) paper was downloaded from the Single Cell Portal of the Broad Institute (singlecell.broadinstitute.org/single_cell). Annotation data for the different cell types and forced-directed layout data were also downloaded. The data was previously size-factor normalized using the computeSumFactors function in R with account to library size. Feature selection, dimensionality reduction, and doublet identification were additionally performed using the scran and scater packages in R, and the data was log-transformed with the scater normalize function. Cells within the dataset were subsequently filtered based on key criteria for “unique features” (>1000 transcripts/cell) and “mitochondrial RNA content” (<20 per cell). These data were loaded into R with cell type data and further analyzed with the Seurat package. FindVariableFeature function was run with default dispersion function and mean functions was used.

Cell Cluster Regrouping. The Seurat function FindAllMarkers function was used to find globally enriched genes within each cell type. Default arguments were used except for a log2fc threshold of 0.5. Data was plotted ordered by log2fc with an adjusted-p-value cutoff of 1×10⁻²⁰. Two highly significant genes (log2fc>5, p-adj<1E-20) were selected based on differential gene expression to represent Gut Tube (GT) (UBA52, RPL38) or the MH (DHX99, HNRNPU) cell populations. The previous cell clusters were readjusted based upon additional clustering of cell that either expressed or did not express with these genes. Cells found with expression values over a standard deviation above expected compared to the cell type for both markers (UBA52 and RPL38 for GT, DHX9 and HNRNPU for MH) were regrouped to either GT or MH respectively.

For re-clustering, statistical analysis was used of 4 highly upregulated markers (two in the GT, two in the HB) to improve clustering (FIG. 6G), and performed a new heat map which had improved clustering (FIG. 1D, FIG. 6H. Table 1). To determine marker expression across clusters, the tools Harmony and Palantir were used and force-directed plots were re-created (FIG. 6I-J). Harmony groups cells by cell type, and Palantir orders cells by pseudo-time and assigns probability for differentiation. The final force-directed plots demonstrate the DE to HB transition (FIG. 6E), the four re-grouping markers (FIG. 6K), liver differentiation genes (FIG. 6F) and major hepatic transcription factors (TFs) (FIG. 6L). The hepatic TFs HEX, TBX3, and PROX1 were nearly exclusively upregulated in the MH population (FIG. 6L). EHT was visualized by comparing EPCAM (Epithelial) to DLK1 (Hepatic) expression. The MH population had high DLK1 expression and low EPCAM expression (FIG. 6L). Since the MH population was tied to growth, the cell cycle was analyzed with a cell phase plot, and found that MH cells, compared to the GT and HB populations, were more actively cycling in the G2-M (mitosis) and S phases (DNA synthesis) (FIG. 6M).

T-distributed stochastic neighbor embedding (TSNE). Principal components were found for all cell types (DE, GT, MH, HB, HM) using the normalized log count data for all of the genes. The first 50 principal components were used to calculate the TSNE coordinates using the Seurat function, RunTSNE. The perplexity was set to 30. Data were graphed with a point size of 5 with Dimplot.

Pathway Heatmaps. Gene Set lists were downloaded from Mouse Genome Informatics (www.informatics.jax.org). The ScaleData function with a negative binomial model was used. The DoHeatmap function was then used with the scaled data and the gene set lists to create heatmaps for each gene set.

Average Heatmap. Average heatmap scores were determined by running the ScaleData function with a negative binomial model for all the genes within the Kegg or GEO biological process pathway. Genes within the dataset were not included in the further analysis. The scaled expression data was then averaged for all the genes in the pathway for each individual cell, and this averaged expression was averaged again for all the cells in each cell type. The colors were then set based on a Red-Green spectrum with a RGB color model. Listed in the scale in each figure, red indicates lower gene expression and green indicating higher gene expression.

Pathway validation. To validate pathways in the MH cluster, the expression of five liver differentiation genes (ALB, AFP, HEX, PROX1, TBX3) were analyzed, and these correlated in all three databases (DAVID, REACTOME, ENRICHR).

Differential Expression Analysis-DAVID. The FindMarkers function in Seurat was used to find differentially expressed genes between different cell types. These gene lists were able to be further filtered for genes with a log2fc>0.5 and an adjusted-p-value less than 0.05. The Entrez gene symbols from these lists were loaded into the DAVID Bioinformatics Resources 6.8 Analysis Wizard. The Functional Annotation Tool was then used to find gene ontologies and pathways with significant enrichment. In DAVID, a Fisher's Exact test is used to measure gene-enrichment for a specific gene set. DAVID produces a p-value from this test, and this p-value is adjusted based on the Benjamini-Hochberg method. Kegg Pathways and Gene Ontology (Biological Processes) were used, and only gene sets with an adjusted p-value<0.3 were used in our analysis and plotted in bar graph format.

Differential Expression-ENRICHR. The same gene lists were used for the ENRICHR analyses as the DAVID analyses. The gene lists for the comparisons between the MH and the GT as well as the MH compared with the HB were used. Both downregulated and upregulated genes were tested separately. The Entrez gene symbols were loaded into ENRICHR. The ENRICHR gene list enrichment analysis tool was used to find significant transcription factors with the ENCODE and ChIP Enrichment Analysis (ChEA) Consensus

TFs. Kegg 2021 Human, WikiPathway 2021, and GO Biological Process 2021 were the gene sets used for the pathway analysis. All data was combined into a single data table, with information about the source of the pathway and whether it was found for the upregulated or downregulated list. These data were then filtered to find gene sets with an adjusted p-value<0.3.

Comparison of DAVID and ENRICHR. DAVID and ENRICHR are able to receive human and mouse genes as input. Both contain gene-set libraries from several sources (Gene Ontology, Kegg, Wiki Pathways, REACTOME, Biocarta, etc.). In addition to ontology and pathway libraries, ENRICHR additionally offers transcription, disease/drugs, cell type, and miscellaneous libraries to further analyze gene lists. Many of these pathways are exclusive to ENRICHR. For enrichment calculations, DAVID uses a modified Fisher Exact Test, called Expression Analysis Systematic Explorer (EASE), which is a more conservative test compared to the Fisher Exact Test. It calculates p-values after subtracting one gene from the List Hits (LH). These p-values were further adjusted with the linear step-up method of the Benjamini and Hochberg (DAVID: www.ncbi.nlm.nih.gov/pmc/articles/PMC2615629/). ENRICHR uses a Fisher exact test, which is corrected with a z-score permutation background correction. This process uses many random input gene lists to compute a mean rank and standard deviation from the expected rank. From this calculation, it is able to calculate a z-score, which is further combined with the p-value to score the pathways (ENRICHR Source: www.ncbi.nlm.nih.gov/pmc/articles/PMC3637064/).

Pie Chart analysis. The FindMarkers function in Seurat was used to find differentially expressed genes between different cell types. These lists were found comparing GT vs. MH and HB, MH vs. GT and HB, and HB vs. GT and MH, for both upregulated and downregulated genes. These Entrez gene lists were loaded separately into the REACTOME 3.7 Analysis Tool. From the REACTOME analysis, the number of genes in each of the highest hierarchical gene type were used to create a pie chart.

Gene expression PCA plots. Gene expression plots were plotted using the FeaturePlot function with the normalized expression data. A blue-red expression was used with blue indicating higher relative expression and red indicating lower relative expression.

Pathway Ranking. A total of 15 comparisons involving single (e.g., MH to HB), double (e.g., MH to GT and HB), triple (e.g., MH to DE, GT, and HB) were performed to develop a list of ranked pathways, focusing on for signaling and metabolism genes. Single comparisons (e.g., MH to HB) only were also performed. For each comparison, an up-regulated and down-regulated list of pathways was obtained (KEGG and/or Biological Process). The comparisons were ranked by the frequency in which each pathway appeared in each comparison performed, and calculated an averaged FDR value for all the comparisons.

Heatmap ranking. A scoring analysis was performed by averaging the expression across all genes in a given pathway, and performing a combined heat map analysis by comparing groups.

Reagents/Materials. RPMI 1640 medium with GlutaMAX (Cat. #: 61870036), Knockout Serum Replacement (KOSR) (Cat. #: 1082810), DMEM, high glucose, GlutaMAX™ Supplement, pyruvate (Cat. #: 10569010), IMDM medium, Cat. #: 31980030, Ham's F-12, Cat. #: 11765054, N-2 supplement, Cat. #: 17502048, L-Glutamine, Cat. #: 25030081, L-Glutamine, (Catalog #: 25030081), Fetal Bovine Serum (Cat. #: A3160701), Penicillin-Streptomycin (P/S) (10000 U/ml) (15140122), B27™ Supplement (50×), serum-free (Cat. #: 17504044), 0.05% Trypsin-EDTA (Cat. #: Cat. #: V22887), Vybrant DiO Cell-Labeling Solution (Cat. #: V22886), Vybrant Dil Cell-Labeling Solution (Cat. #: V22885), L-Ascorbic acid, (Cat. #: A61-25), were purchased from Thermofisher. mTESR1 medium (Cat. #: 85850), Accutase (Cat. #: 07920), Dispase (Cat. #: 07923), Gentle dissociation reagent (Cat. #: 100-0485), Y27632 (ROCK) Inhibitor (Cat. #: 72304), Human Recombinant Activin (Cat. #: 7800.1). Dexamethasone (Cat. #: 72092), Human Recombinant Growth Factor (HGF), (Cat. #: 78019.1), Human Recombinant Oncostatin (M)), (Cat. #: 78094), KGF (Human Recombinant FGF-7), (Cat. #: 78046), bFGF (Human Recombinant), (Cat. #: 78003.1) were purchased form StemCell Technologies. N2 Supplement (100×) (GIBCO, Cat. #: 17502001), CHIR99021 (Cat. #: SML1046-5 MG), EGM™-2 Endothelial Cell Growth Medium-2 Bulletkit™ (Lonza, Catalog #: CC-3162), MG (Growth factor-free) (Cat. #: 354230), Collagen, rat tail, (Cat. #: 354236) was purchased from Corning. Aurum Total RNA Mini Kit (Cat. #: 7326820), DNase I (Cat. #: 7326828), iTaq Universal SYBR Green Supermix (Cat. #: 1725121), and iScript cDNA Synthesis Kit (Cat. #: 1708891), 96-well PCR plates (Cat. #: L223080) were purchased from Bio-Rad Vybrant DiD (red, Invitrogen, Cat. #V22887), Dil (yellow, Invitrogen, Cat. #V2885), or DiO (green, Invitrogen, Cat. # V22886), Tissue Culture Treated 24-well plate (LPS, Cat. #: 702001), 75 cm² Polystyrene tissue culture-Treated Flasks (LPS, Cat. #: 708003), 60 mm tissue culture treated dishes (LPS, Cat. #: 705001), 384-well round bottom, ultra-low attachment spheroid microplates (Corning, Cat. #: 3830), 96-well Cell Culture Plate (LPS, Cat. #: 701001), 6-well Cell Culture Plate (LPS, Cat. #: 703001), 96-well PCR plates (LPS, Cat. #: L223080), PCR Plate Covers (LPS, Cat. #: HOTS-100). Shifferdecker Staining Jar (Catalog No: 70314-05, Electron Microscopy Services), Slide rack (Cat. #: 70312-24, Electron Microscopy Services), Tissue Tek Base Molds for Embedding Rings (Cat. #: 62527-38,4124; Electron Microscopy Services), Millenia 2.0 Adhesion Slides, 75×25 mm (Cat. #: 71863-01, Electron Microscopy Services), Microprocessing/Embedding Cassettes (Cat. #: 70073-B, Electron Microscopy Services), Eosin Y (Cat. #: DcE-40, Electron Microscopy Services), Hematoxylin (Catalog No: DcH-48, Electron Microscopy Services), Permount™ Mounting Medium (Cat. #: 17986-01, Electron Microscopy Services), Cover-slips (Cat. #: 12-542-AP, Fischer Scientific). All primers for qRT-PCR were purchased from either Integrated DNA technologies (IDT), Sigma Aldrich, or Thermofisher. The following small molecule pathway inhibitors were ordered: LDN193189 Hydrochloride, Sigma, Cat. #: SML0559-5 MG, SB431542, StemCell Technologies Cat. No: 72232, Y-27632 2 inhibitor (ROCK Inhibitor), MyBiosource, Cat. #: MBS577605; Crizotinib (PF-02341066), Selleck Chem Cat. #: S1068, Wortmannin, Selleck Chem, Cat. #: S2758, SU5416, Cayman Chemicals, Cat. #: 13342; A83-01, Stemgent, Cat. #: 04-0014, Verteporfin, Sigma, Cat. #: SML0534-5 MG. Bovine serum albumin (BSA), Cat. #A9576-50ML, and 1-Thioglycerol, Cat #: M1753-100ML were purchased from Sigma.

Cell lines. iPSC (induced pluripotent stem cell) cell line: ATCC-BXS0114 Human (African American Female) Induced Pluripotent stem cells (iPSC, ACS-1028™). Embryonic stem cell line: UCSF4 (human embryonic stem cell (hESC), female, NIH Registry (0044), University of California San Francisco (UCSF). HepG2 liver carcinoma cells (ATCC®, Cat. #: HB-8065). Human umbilical vein endothelial cells (HUVEC) (Lonza®, Cat. #CC-2935). Human foreskin fibroblasts (HFFs) were obtained as a kind donation from Dr. Stelios Andreadis (University at Buffalo).

Antibodies. Mouse anti-human AFP monoclonal antibody (Cat. #: sc-130302, Santa Cruz Biotechnology). Rabbit anti-human albumin (Alb) monoclonal antibody (Cat. #: 109-4133, Rockland). Mouse anti-human CDX2 monoclonal antibody (Cat. #: sc-393572, Santa Cruz Biotechnology). Mouse anti-human SOX2 monoclonal antibody (Cat. #: sc-365823, Santa Cruz Biotechnology). Mouse anti-human CD31 (PECAM-1) monoclonal antibody (Cat. #: sc-71872, Santa Cruz Biotechnology). Mouse anti-human Foxa2 monoclonal (Cat. #: MA5-15542, Thermo Fisher). Mouse anti-human HNF4a monoclonal antibody (H-1, Cat. #: sc-374229, Santa Cruz Biotechnology). Mouse anti-human SMA monoclonal antibody (Cat. #: sc-53015, Santa Cruz Biotechnologies), Mouse anti-human TBX3 monoclonal antibody (Cat. #: sc-166623, Santa Cruz Biotechnologies). Rabbit IgG (Cat. #PI31235, Thermofisher). Mouse IgG2a kappa (Cat. #5013049, Thermofisher). Mouse IgG2b kappa (Cat. #5013052, Thermofisher).

Feeder-free culture (maintenance), harvesting, and collecting of hPSC. Methods are as described previously.

Culture of cell lines (HepG2, HUVEC, HFF). HepG2 hepatoblast carcinoma cells (ATCC HB-8065) and human foreskin fibroblast (HFF) cells (ATCC SCRC-1041) were passaged at a 1:10 dilution on tissue culture-treated T-75 flasks in high glucose DMEM

(Thermofisher) containing 10% FBS (ThermoFisher) and 1% P/S. Medium was changed every other day. HUVEC (Lonza) were cultured in complete EGM-2 (Lonza) medium containing 1% P/S, with medium changed every other day. Modified EGM2, as described below, was used for stem cell experiments.

In vivo transplantation assay. The transplant procedure was based upon prior studies. 1×10⁶ control hPSC and hPSC-derived DE combined with Human foreskin fibroblasts (HFF) (4:1 ratio of DE: HFF), and HEPG2 (human liver cancer), was harvested from in vitro monolayer culture using the appropriate dissociation reagent, washed in cell-appropriate medium, and collected in microcentrifuge tubes. hPSC-derived cells were transplanted in 8-10 week-old female immunodeficient NOD-SCID mice (Jackson Laboratories). Mice were anesthetized and maintained with inhaled isoflurane (1-3%). hPSC-derived cells were directly mixed on ice with 50 μL of growth factor free Matrigel (MG), or mixed with HFF and then mixed with MG. The MG/cell mixture was then transplanted subcutaneously in the left hindlimb of NOD-SCID female mice using a 24-gauge needle. After cell transplantation, mice were kept under isoflurane for a few minutes to allow the cell/gel combination to solidify in vivo. Animals were recovered, and resumed spontaneous respiration on temperature-controlled heating pads. Mice were monitored carefully. After 4 weeks, mice were sacrificed for tissue assessment as described below.

Design of culture system. Since the unique transplantation model supported the hLD-MESC hypothesis, an in vitro system based on this was developed (FIG. 1A-C). The focus on early LO precluded the use of traditional instructive/maturating factors like HGF, oncostatin, dexamethasone. Further, it was noted that sequential instructive factors can provide mixed signals to a population of differentiating cells, for example, potentially signaling proliferation in some cells and alternate cell fates in others, and thus it was chosen to eliminate sequential factor design. Instead, a single medium formulation was chosen, without any changes, which was employed for the entirety of differentiation under hypoxic conditions. In the first condition, a nutrient rich medium was chosen containing, SFD previously used for maintaining hPSC-derived endoderm progenitor cells, the precursor to HBs, with minimal growth factors. KGF was retained (FGF7) in the medium, since KGF has been shown to induce hPSC-differentiation of endoderm towards GT. For the second strategy, data from ENRICHR pathway analysis was used (Table 7) to identify that VEGF, EGF, FGF2, and IGF-1 signaling were upregulated in the MH population. Each of these factors has been linked to liver differentiation and growth. It was found that EGM-2 medium, a commercially available medium employed VEGF, EGF, IGF-1, and FGF (FIG. 2G), and therefore EGM-2 medium was employed, but removed both hydrocortisone and serum (FIG. 2G). Molecular control of liver development suggested that liver genes are primed to activate even within day 6 hPSC-derived GT endoderm. This molecular control concept supports the idea that spontaneous hepatic differentiation can occur from endoderm in the absence of formal instructive factors.

Preparation of SFD medium. Methods are as described previously.

Preparation of H+M medium. Base (control) medium contained RPMI, 1% B27, 0.2% Knockout serum (KOSR), and 1% P/S. The modified EGM-2 medium contained EGM-2 basal medium (Lonza), 2% KOSR, 0.5% B-27, 0.05% long R3 insulin growth factor (R3-IGF), 0.05% epidermal growth factor (EGF), 0.2% fibroblast growth factor 2 (FGF2), 0.05% vascular endothelial growth factor (VEGF), 0.05% ascorbic acid, 0.05% heparin, 0.05% gentamycin, 1% P/S. The absolute concentrations of growth factors and heparin within EGM-2 are not known. Hydrocortisone and fetal bovine serum (FBS) were not added to the modified EGM-2, medium because of their complex effects on cells, and the FBS was replaced with KOSR (Stemcell technologies) in the modified EGM-2 medium, which is a defined serum. The base medium and the modified EGM-2 medium was mixed at a ratio of 1:1 and this was called Hepatic and Mesenchymal medium (H+M medium).

DE induction from human stem cells under hypoxic (5% O₂) conditions. Methods are as described previously. SFD medium contains RPMI supplemented with 75% IMDM, 25% Ham's F12, 0.5% N2 Supplement, 0.5% B27 supplement, 2.5 ng/ml FGF2, 1% Penicillin+Streptomycin, 0.05% Bovine Serum Albumin, 2 mM Glutamine, 0.5 mM Ascorbic Acid, and 0.4 mM Monothioglycerol (MTG).

HB induction using growth factor GF(+) differentiation protocol. A hepatic differentiation protocol was adopted with an additional GT endoderm stage. Under hypoxic conditions (5% O₂) until day 14, 2×10⁵ DE cells were seeded per well of a 24-well plate. Following DE induction by day 4, cells were induced to form GT endoderm using SFD media supplemented with 25 ng/mL (KGF/FGF-7). On day 6 of culture after GT induction, cells were treated in SFD medium with bone morphogenetic protein 4 (BMP4, 10 ng/ml), and fibroblast growth factor 2 (FGF2, 20 ng/ml) for specification of liver cell fate. On days 10-14, cells were treated with hepatocyte growth factor (HGF, 10 ng/ml), oncostatin (20 ng/ml), and dexamethasone (100 nM). 500 μL of culture media was used for daily medium changes.

HB cell induction using GF(−) containing SFD medium. Under hypoxic conditions (5% O₂) until day 14, hPSC-derived DE progenitor cells derived on day 4 were induced to early HBs using a protocol in which no additional GF are added, to enable spontaneous hepatic differentiation. Briefly DE progenitor cells were incubated with SFD medium supplemented with (KGF/FGF7) at 25 ng/ml (in addition to FGF2 (2.5 ng/mL), from day 4-14. Differentiation throughout this protocol utilized 24-well plates and 500 μL of culture medium for daily medium changes.

HB cell induction using hepatic and mesenchymal (H+M) medium. hPSC-derived DE were differentiated towards HBs from day 4 to day 14 using H+M medium (defined above), under hypoxic conditions (5% O₂). 2×10⁵ DE cells were seeded per well of a 24-well plate. The protocol used daily medium changes (500 μL per well). 500 μL of culture medium was used for daily medium changes in each well of a 24-well plate.

Fluorescent dye-labeling of stem cell-derived progenitors. hPSC-derived progenitor cells at different stages of differentiation were harvested (500 μL/well Accutase, 5 minutes, 37° C.) and adjusted to a final concentration of 1×10⁶ cells/mL appropriate medium. Subsequently, 5 μL of either Vybrant DiD (Invitrogen), Dil (Invitrogen), or DiO (Invitrogen) cell-labeling solution was added to 1 ml of cell suspension in a microcentrifuge tube, incubated for 20 minutes at 37° C., centrifuged at 1500 RPM for 5 minutes. The remaining cell pellet was then washed twice in fresh culture media before being seeded for liver organoid formation.

Live/dead assay for thin cell sheet and liver organoids. Organoids were assayed for cell viability using a live/dead reagent kit (Biotium, Catalog #30002-T). Briefly, kit reagents consisted of stock reagents: Calcien AM (live) and Ethidium bromide (EthD-III, dead) which were warmed to room temperature before being diluted to 2 μM and 4 μM respectively in serum-free DMEM containing 1% P/S. Organoids were rinsed once with PBS and then incubated for 3 hours in live/dead media reagents (37° C., 5% O₂). Tissues, cultivated in incubation medium are then imaged for viability under green (live cells) and red fluorescence (dead cells) which will indicate cell viability. Images are obtained under fluorescence using Zeiss Axiovision fluorescence microscope (Observer Z.1).

GT organoids. GT endoderm organoid formation. To obtain GT organoids, hPSCs-derived DE (day 4) cells were differentiated on feeder-free, MG-coated plates (1:15 dilution in high glucose DMEM) under hypoxic conditions (5% O₂) until day 6 in SFD medium. Next, differentiation medium was aspirated from each well and 500 μL of Accutase (Stemcell technologies) was added to each well and incubate for 5 minutes at 37° C., collected into a 15 mL tube, and centrifuged at 1000 RPM for 5 min. The medium containing Accutase was then aspirated, and cells were washed in fresh medium, and re-suspended in SFD medium containing ROCK inhibitor (Stemcell technologies) (1:1000) and counted. The total number of cells needed per well was 2×10⁴ so adjustments were done to have an appropriate number of cells for seeding. Prior to seeding cells for each organoid within each well, cells were mixed to maintain an appropriate cell distribution. GT cells were collected 50 μL and seeded into a number of wells of a sterile 384-well round bottom ultra-low attachment plate (Corning). The plate was centrifuged at 1000 RPM for 10 minutes in order to properly collect cells at the bottom center for improved compaction and organoid formation. Plates were incubated in 37° C. under hypoxic conditions (5% O₂) for 24 hours until organoids formed and imaged after each 24 hours using phase contrast and fluorescent microscopy. Organoids were further cultured and processed depending on the application.

GT endoderm/HUVEC organoid formation. On day 6, GT cells were harvested as described herein. HUVECS (previously cultured in complete EGM-2 medium) were dissociated using trypsin (0.25%) at 37° C. for 5 minutes. Both cell types were separately collected into a 15 mL tube, centrifuged at 1000 rpm for 5 min, counted, and resuspended in SFD. The ratio of GT to HUVEC cells 1:1, therefore 1×10⁴ cells, of each cell type per well were used for organoid formation in a 1:1 mixture of SFD:EGM-2 culture medium. The mixture of cells was seeded as described herein, and organoids formed within 24 hours.

GT organoids in EGM-2 medium. Day 6 GT endoderm cells were obtained as described herein. After Accutase digestion and harvest, the pellet was washed in fresh medium and resuspended in full EGM-2 (Lonza) medium. The collected cells were seeded (50 μL/well) at a density of 2×10⁴ cells/well into a 384-well round bottom ultra-low attachment plate, and centrifuged to compact the cells into organoids, which formed in 24 hours, and further cultured in EGM-2 medium under hypoxic conditions (5% O₂).

Generation of microfabricated device (micropillar device) for functional analysis of mesenchymal components. A micropillar device was fabricated using a multi-layer microlithography technique. SU-8 masters were generated via spin coating, alignment, and then subsequent exposure and baking of multiple layers of SU-8 photoresists. To obtain the cross-sectional difference between the micropillar head and leg sections, a thin layer of SU-8 doped with S1813 was deposited to prevent UV light penetration to the leg section. Following this step, UV exposure was performed on an OAI mask aligner that encompassed a U-369 band pass filter. PDMS (Sylgard 184, Dow Corning) stamps were casted over the SU-8 master at 10:1 mixing ratio to demold the micropillar pattern. Additional micropillar devices were produced through replica molding from stamps in P35 petri dishes or 12 well plate. The devices were then prepared for appropriate cell seeding.

Generation of thin organoids (microtissues) in microfabricated device for functional analysis of mesenchymal components. Micropillar devices were sterilized and subsequently treated with Pluronic F-127 to minimize non-specific cell adhesion to a PDMS surface. For HepG2 cells alone, a total of 1×10⁶ cells were used in complete DMEM (cDMEM) containing high glucose DMEM (Thermofisher) 10% FBS, 1% P/S. For HepG2: HUVEC microtissues, a 1:1 mixture of HepG2 cells and HUVEC cells was used (total cell number 1×10⁶), in a 1:1 mixture of cDMEM:EGM-2 medium. In the case of HUVEC cells, a total of 1×10⁶ cells were used in complete EGM-2 medium. For the hPSC-derived H+M cell cultured organoids were collected from 24 well plates (500 μL/well Accutase digestion, 5 minute, 37° C.) and adjusted for 1×10⁶ cells per tissue device. All cell compositions were then mixed with collagen type-I at a final concentration of 3 mg/mL and introduced to the microwells via centrifugation. The collagen solution was then allowed to crosslink and maintained in 3 mL of complete H+M media in a CO₂ incubator. The contraction force generated individual microtissues is as determined by the cantilever bending theory, where El is the clastic modulus, L is the length of the cantilever, and δ is the distance the cantilever top moved. Micro-pillar deflection was measured using phase contrast microscopy by comparing the deflected position of the centroid of each pillar top to the centroid of its base. HepG2 or HepG2: HUVEC tissue devices, hPSC-derived tissues were cultivated at in 37° C., and 5% CO₂, 5% O₂.

Preparation of agarose-coated microwells. Sterile, 1 wt % agarose (1 g/100 mL distilled H₂0) was prepared, heated to liquid form, and pipetted (50 μL/well) into a 96-well plate (Corning). The plate was then allowed to be cooled (25° C. for 20 minutes) prior to cell seeding.

Collagen type I gel formation. Rat tail collagen type I was provided at a concentration between 3-4 mg/mL in 0.02N acetic acid (Corning; Catalog #: 354236). Current available lot of Rat tail collagen Type I (Corning; Lot #0048003) was constituted at a concentration of 3.78 mg/mL. A working concentration of 2 mg/mL Rat tail collagen type I was utilized for downstream experiments. Adhering to manufacturer instruction, a total desired volume of 5 mL of 2 mg/ml collagen gel (CG) was prepared with pre-calculated amounts of de-ionized water, 10×PBS, and 1N NaOH. For the 3.78 mg/ml of stock rat tail collagen type I to polymerize, equivalent moles of NaOH are needed to neutralize the 0.02N acetic acid solvent (determined to be 60.8 μL of 1 N NaOH for 2.64 ml of 3.78 mg/mL rat tail collagen in 0.02N acetic acid needed to make 5 mL of 2 mg/mL CG). The osmolarity of the CG gel solution was adjusted with 0.5 mL of 10×PBS (pH 7.4) resulting in a final 1× concentration after gel preparation. De-ionized water was used to adjust the remaining solution to the total desired volume (calculated to be 1.80 mL for 5 mL of 2 mg/mL CG). The reagents were sterilely prepared and added to a 15 mL centrifuge tube on ice in the following order: 1.80 mL de-ionized water, 0.5 mL 10×PBS, 60.8 μL of 1 N NaOH followed by 2.64 mL of 3.78 mg/mL stock rat tail collagen type I in 0.02N acetic acid. The contents were briefly vortex before use in downstream applications. Prepared collagen can be stored at 4° C. for 4 days.

Organoid culture in MG droplets. Twenty, day 15 control and H+M (twenty organoids, 300-400 μm in diameter) were collected from suspension culture and seeded onto 60 mm tissue culture treated dishes. Organoids were first collected in a 15 mL tube on ice from organoid suspension culture in well plates. The organoids were allowed to settle and the medium in the tube was aspirated (on ice). Separately, 1 mL of ice-cold diluted MG (growth factor free) mixed with RPMI control medium, 1:1) was mixed with the organoids. The organoid/MG suspension was then mixed and distributed evenly inside the MG solution. To form MG droplets containing organoids, using a 200 μL pipette, a 15 μL volume of one organoid/MG solution is collected and seeded onto a 60 mm dish, for a total of 15-20 droplets. If more than organoid is seeded per droplet, it is removed and reseeded appropriately. Organoid/MG solutions are pipetted slowly onto the 60 mm dish to avoid air bubbles. Then, the droplets were incubated at 37° C., 5% O₂ for 30 minutes to allow the MG to solidify before culture in H+M medium, of which 5 mL is slowly added to the dish and changed every 3 days. Organoids were imaged daily on the 60 mm dish using a (EVOS fluorescent, phase contrast microscope, #AMEFC4300R) at 4×, 10×, and 20× magnification.

Organoid culture in collagen droplets. Organoid droplet culture is the similar to the above except 1 mL of ice-cold rat tail collagen Type 1 (2 mg/ml) is employed instead of MG.

Harvesting spheroids embedded in MG or collagen. Spheroids embedded in gel were harvested and placed in 15 mL tubes. Accutase was then added to the tubes. The tube was placed in the cell incubator to allow the spheroids to detach from gel for 1 hour. If processing for protein or gene expression, spheroids were washed with PBS and then resuspended in 0.25% trypsin for an additional hour to break down spheroids into cells. Cells were then washed with PBS again before being processed for protein or mRNA isolation.

Histology assay. Organoids were fixed in 10% neutral buffered formalin for 30 minutes before being washed once in 70% ethanol and then processes for paraffin embedding, embedded in agarose (2 wt %) prior to paraffin embedding. Paraffin embedded blocks were then sectioned at 10 μm per tissue section. Antigen retrieval was completed by heating rehydrated section in 1× Tris-EDTA buffer solutions for 20 mins in a microwave. In addition, paraffin embedded, 10 μm sections, were also stained with Eosin and Hematoxylin (Electron Microscopy Services) and mounted with medium before microscopy (Zeiss Axiovision fluorescence microscope (Observer Z.1)).

Histological analysis of organoid and tissues. Organoids were fixed in 10% neutral buffered formalin for 30 minutes before being washed once in 70% ethanol and then processes for paraffin embedding, embedded in agarose (2 wt %) prior to paraffin embedding. Paraffin embedded blocks were then sectioned at 10 μm per tissue section. Antigen retrieval was completed by heating rehydrated section in 1× Tris-EDTA buffer solutions for 20 mins in a microwave. In addition, paraffin embedded, 10 μm sections, were also stained with Eosin and Hematoxylin (Electron Microscopy Services) and mounted with medium before microscopy (Zeiss Axiovision fluorescence microscope (Observer Z.1)). Harvested tissues were collected and immediately fresh frozen in dry ice. Samples were then quickly thawed before being formalin fixed (neutral buffered formalin 10% overnight). Samples were then processed for embedment in paraffin wax using standard techniques. (Histology Core, Jacobs School of Medicine and Biomedical Sciences). Tissues were then sectioned at 5 μm and stored until further processing. For hematoxylin and cosin staining, tissue sections were deparaffinized and stained using standard procedures. For organoid histological analysis, samples were fixed in 10% neutral buffered formalin for 30 minutes before being washed once in 70% ethanol and then embedded in agarose (2 wt %) prior to paraffin embedding (Histology Core, Jacobs School of Medicine and Biomedical Sciences). Paraffin embedded blocks were then sectioned at 10 μm per tissue section (Histology Core, Jacobs School of Medicine and Biomedical Sciences). In addition, paraffin embedded, 10 μm sections, were also stained with Eosin and Hematoxylin (Eosin Y Catalog Number (DcE-40), Hematoxylin Catalog Number (DcH-48)) in a similar method to whole tissues and covered with mounting medium before microscopy (Zeiss Axiovision fluorescence microscope (Observer Z.1)).

Microscopy of organoids. For cellular imaging, organoids placed inside 384-well plates were imaged within droplets for both phase contrast and fluorescence microscopy using a Zeiss Axiovision fluorescence microscope (Observer Z.1) using 4×, 10× objectives.

Immunofluorescence microscopy. For determination of intracellular localization of ALB, AFP, FOXA2, or other proteins, cells cultured on tissue culture treated dishes were washed once with PBS and then fixed with 10% neutral buffered formalin, permeabilized with 0.1% Triton X-100 in PBS, and blocked with 1% BSA in PBS for 30 minutes. Plates were then incubated with primary antibodies overnight, rinsed with PBS and incubated with secondary antibody (AlexaFlour 488, Thermofisher, 1:1000 dilution) for one hour, before co-nuclear detection with DAPI (4,6-diamidino-2-phenylindole) and followed by fluorescence microscopy. Controls were non-specific antibody and secondary antibody only.

Whole organoid fixation and immunofluorescence. An additional staining protocol was developed for whole mount liver organoids in suspension. Briefly, liver organoids were fixed in 10% neutral buffered formalin for 1 hour and then blocked for 2 hours in 1% BSA. The organoids were then incubated with primary antibody (1:100) at 4° C. overnight. The following day, organoids are washed 3 times with 1% PBST (each wash 20 mins) under gentle agitation at room temperature. Secondary antibody (AlexaFlour 488, Thermofisher, 1:1000 dilution) was then added for incubation at 4° C. overnight and washed out as described above. DAPI incubation (10 minutes) was used to counterstain before images were obtained. Controls were secondary only and nonspecific IgG subtype with secondary antibody. Phase contrast and fluorescence microscopy were obtained using a Zeiss Axiovision fluorescence microscope (Observer Z.1).

Urea assay. Urea metabolite concentration analysis was done via a spectrophotometric urea assay kit (Bioassay systems, Catalog No: DIUR-100). Urea metabolite reacts directly with assay kit containing urease. Urease catalyzes the breakdown of urea into ammonia and carbon dioxide. The generate ammonia reacts turns blue in the presence of the assay reagent kit containing hypochlorite. The more urea, the bluer the sample. Per assay guidance for in vitro cell culture media extract, 50 μL of each cell culture supernatant was seeded into a 96 well tissue culture treated plate. For accurate analysis, a correlating blank of 50 μL of fully supplemented H+M cell culture media was used. In addition, the standard (50 μL) was also pre-diluted in H+M media for low urea concentration analysis. All analyzed samples including standard and blank were seeded in triplicate. Then 200 μL of working regent (Reagent A+Reagent B, 1:1) was introduced into each well. Subsequently, the plate was incubated at room temperature for 50 minutes before absorbance was read at 450 nm for optical density (O.D.) in a Biotek Synergy 4 Multiplate reader. The standard and the blank were used in an interpolation equation to determine urea concentrations from sample absorbance values obtained from the spectrophotometer analysis.

ELISA assay. To determine secreted ALB protein, sandwich ELISAs were performed using cell culture medium samples that were collected at different time points, in addition to standard samples of known concentrations. To coat the plate, captured antibody (monoclonal mouse anti-human albumin antibody—Abcam, Inc) was diluted to 2 μg/mL in 1×PBS. 100 μL of the diluted antibody was added separately to each well of a 96 well EIA/RIA high binding plate (Laboratory product sales (LPS)), and the plate was incubated overnight at 4° C. with gentle shaking. The next day, the wells were washed three times with 100 μL of PBST (PBS containing 0.5% Tween-20) for 5 minutes each wash. Then 200 μL of blocking buffer (1% bovine serum albumin (BSA)) was added to each well to block residual protein-binding sites, the plates being incubated for 1 hour at 37° C. Next, the wells were washed three times with PBST for 5 minutes each time. After that, 100 μL of the culture medium samples and controls (DMEM medium only and diluted albumin standards) were added to the appropriate wells before incubating for 1 hour at 37° C. Then the wells were washed three times with 100 μl of PBST for five minutes each time. Then, 100 μl of the detection antibody solution (dilution 2 μg/ml of the biotinylated polyclonal goat anti human albumin antibody in 1×PBS, Abcam, Inc) was added to each well before incubating at 37° C. for an hour. The wells were washed three times with 100 μl of PBST for 5 mins each and 100 μl of the secondary antibody solution (dilution 2.5 μg/ml of the HRP-Streptavidin conjugated antibody in 1×PBS, Abcam, Inc.) was added to each well before incubating at 37° C. for 1 hour. The wells were washed three times with PBST then 100 μl of TMB substrate was added to each well before wrapping the plate in foil and incubating it at room temperature for 30 minutes in the dark. Finally, 50 μl of stop solution was added to each well and the absorbance values were measured at 450 nm (Biotek Synergy 4 Multiplate reader). ALB concentration was determined by creating a standard curve from standard samples absorbance and their respective concentrations. An equation of best fit was then created from the generated curve and used to determine sample concentrations based on respective absorbance values.

Reverse transcriptase reaction and real-time polymerase chain reaction (RT-PCR). Primers are listed in table format. For each experimental condition, cell lysates were collected using the Aurum Total RNA Mini Kit (Biorad). Total RNA was isolated from duplicate or triplicate samples and concentrations were measured using the NanoDrop One/One Microvolume UV-Vis Spectrophotometer, (Catalog #: ND-ONE-W). For reverse transcriptase cDNA was synthesized using the iScript cDNA synthesis kit (Biorad) and an Eppendorf 5331 MasterCycler Gradient Thermal Cycler with 5 ng of RNA for each planned qRT-PCR reaction. The RT temperature protocol was 25° C. for 5 min., 46° C. for 20 min., 95° C. for 1 min., and then either stored at 4° C. or plated at 12° C. prior to plating. For the PCR amplification reaction, each sample was plated in triplicate in a 10 μL per well reaction volume composed of 5 μL of iTaq™ Universal SYBR® Green Supermix, and forward and reverse primers at a concentration of 300 nM. The qRT-PCR reactions were run for 40 cycles (C1000 Touch Thermal Cycler, Biorad). Gene expression analysis was conducted utilizing the delta-delta-Ct method, with GAPDH used as a normalization. The PCR temperature amplification step was as follows: 98° C. for 30 seconds, 98° C. for an additional 15 seconds, 60° C. for 30 seconds and then the process was repeated at steps 2 and 3 until 40 complete cycles had been reached. Subsequently, there was an incremental heating stage from 65° C. to 95° C. at an increment of 0.5° C. for 5 seconds. The plate was then analyzed for relative cycle threshold (CT) value per gene of interest. All primers were purchased from either Integrated DNA technologies (IDT), Sigma Aldrich, or Thermofisher (18-22 bp in size). To quantify gene expression, CT values were calculated for the experimental and control conditions, for both the gene of interest and the housekeeping gene (GAPDH).

The 2^{-ΔΔCT} method was employed for quantification. For each gene, if the CT value after the PCR reaction was not detected, then 40 was chosen as the CT value reflecting the largest possible CT value using our PCR reaction. The delta-delta CT quantification method was then carried out.

Analysis of organoid migration. ImageJ was used to identify relative characteristics of the migrating organoids. Briefly, images were uploaded into Image J and a scale bar was set for each image uploaded before analysis. The length application in Image J was used to identify the protrusion length and thickness in the various experimental designs. The count plugin application was used to estimate the number of cords in different fields of view and over time. The trace plugin application in ImageJ identified the outline of the edge of the spheroids for overall growth kinetics. Lastly, Skeletonize3D application in image J analyzed the branching phenotype observed in migrating spheroids. With regards to Skeletonize3D plugin, images were first transformed into 8 bit grey-scale, before being thresholded to denote the peripheral edge of the organoid, and then endpoint image branching analysis performed.

Filtering of lateral 3D migration for improved image visualization. The raw images were loaded into the image editor GIMP 2.10.24. The brightness was increased by 20%-40% based on the brightness level of the spheroid and the contrast was reduced by 30%-40% to decrease the visibility of the background cells and bring out the finer details of the spheroid. The Foreground Select Tool was then used to select the spheroid so the background can be completely removed.

Chemical screening of growth inhibition in MG culture. Eight small molecule inhibitors were employed. As mentioned above, D15 H+M (20 organoids, 300-400 μm in diameter) were collected from suspension culture and seeded onto 60 mm tissue culture treated dishes as done in the MG organoid droplet culture (one organoid per droplet, 20 droplets). After the MG gelling step (droplets were incubated at 37° C., 5% CO₂, 5% O₂ for 30 minutes at to allow the MG to solidify) the organoid droplets were cultured in 5 mL of H+M media supplemented with a pre-determined concentration of a small molecule inhibitor. All inhibitor/H+M solutions were prepared the same way: the stock small molecule inhibitor was thawed from −20° C. storage and diluted directly into H+M medium at room temperature. The inhibitor supplemented H+M media (5 mL) was then added directly to the 60 mm dish containing the MG/organoids and re-incubated (37° C., 5% CO₂, 5% O₂) for an additional 4 days. Organoids were imaged daily on the 60 mm dish using a (EVOS fluorescent, phase contrast microscope, #AMEFC4300R) at 4×, 10×, and 20×. Collective cell migration was then observed over the 4-day period.

The readout of growth and migration was quantitatively assessed by light microscopy. A schematic of our screening approach to screen for growth/migration inhibitors is shown (FIG. 5A). Day 15 organoids in droplet culture were treated with a single small molecule inhibitor and cultured to day 18 compared to untreated controls. Phase contrast microscopy was performed on days 16-18, and growth/migration was quantified. The first criterion for determining inhibition was to demonstrate a block of growth/migration over time, by demonstrating no significant difference between day 16 to day 18. The second criterion was comparing day 18 levels of growth at a higher inhibitor concentration to a lower inhibitor concentration, to determine if there was a significant difference, indicating a block in the observed increase in growth, at the higher concentration. The third criterion was cell viability, since a loss of viability could lead to cell death, which would result in loss of growth (FIG. 5A), and therefore conditions were removed in which a loss of growth was due to a loss in viability. The following were tested: A83-01 (Activin, TGF-β, Nodal, 2-200 μm), SB43152 (Activin, TGF-β, Nodal, 0.1-10 μm), Crizotinib (c-MET and ALK inhibitor, 2.5-250 nm), LDN 193189 (BMP, 50 nm-5 μm), SU5416 (VEGFR2, 0.1-10 μm), Verteporfin (VT) (Hippo (YAP inhibitor, 0.1-10 ug/mL), Wortmannin (PI3K inhibitor, 1-100 nm), and Y27632 (Rho-associated kinase (ROCK), 1-100 μm).

Live/dead assay for biologically inhibited organoids in MG culture. Hepatic organoids cultured in H+M medium and MG droplets until day 18 as mentioned above were assayed for cell viability using a live dead reagent kit (Biotium). Briefly, kit reagents consisted of stock reagents: Calcein AM (live) and Ethidium bromide (EthD-III, dead) which were previously warmed to room temperature before being diluted to 2 μM and 4 μM respectively in serum free culture media containing 1% pen/strep. The 60 mm dishes containing the day 18 H+M organoids are rinsed once with PBS and then incubated for 3 hours in live/dead media reagents (37° C., 5% CO₂, 5% O₂). Afterwards, organoid droplets kept in the incubation media are then imaged for viability under green (live cells) and red fluorescence (dead cells) which will indicate cell viability. Images are obtained under fluorescence using Zeiss Axiovision fluorescence microscope (Observer Z.1).

Statistics. For statistical assessment between two groups, Student's t-test was done (type II with two tails), and p-values equal to or less than 0.05 was considered to have statistical significance and are defined within the text and figures/figure legends. ANOVA was employed for comparison of multiple groups

Transcriptome analysis highlights coordinated upregulation of signaling, CCM, and metabolic pathways in E9.5 migrating hepatoblasts. It was wished to elucidate the dynamics of global transcriptomic changes in early liver organogenesis (E7.5-10.5), including definite endoderm (DE, E7.5), gut tube endoderm (GT, E8.5), migrating hepatoblasts (MH, 9.5), hepatoblasts (HB, E10.5), and hepatomesenchyme (HM, E10.5). The initial quality control analysis (FIG. 6A-B) and TSNE (t-distributed stochastic neighbor embedding) analysis demonstrated clear separation of the cell populations (FIG. 6C), and further analysis demonstrated clear differences between the GT. MH, AND HB conditions (FIG. 6D). However, when heat map analysis was performed for the most highly differentially expressed genes and used prior clusters, it was observed that many cells with expression levels not matching their assigned cluster but showing patterns more similar to other clusters (FIG. 6E-F).

A series of steps was performed in order to re-cluster the data. Statistical analysis of 4 highly upregulated markers (two in the GT, two in the HB) were used to improve clustering (FIG. 6G), and performed a new heat map which had improved clustering (FIG. 1D, FIG. 6H, Table 1). To determine marker expression across clusters, the tools Harmony and Palantir were used, and re-created force-directed plots (FIG. 6I-J). Harmony groups cells by cell type, and Palantir orders cells by pseudo-time and assigns probability for differentiation. The final force-directed plots demonstrate the DE to HB transition (FIG. 1E), the four re-grouping markers (FIG. 6K), liver differentiation genes (FIG. 1F) and major hepatic transcription factors (TFs) (FIG. 6L). The hepatic TFs HEX, TBX3, and PROX1 were nearly exclusively upregulated in the MH population, as expected (FIG. 6L). EHT was visualized by comparing EPCAM (Epithelial) to DLK1 (Hepatic) expression. The MH population had high DLK1 expression and low EPCAM expression (FIG. 6L). Since the MH population was tied to growth, the cell cycle was analyzed with a cell phase plot, and it was found that MH cells, compared to the GT and HB populations, were more actively cycling in the G2-M (mitosis) and S phases (DNA synthesis) (FIG. 6M).

Since the re-clustering was successful, the transcriptomics of up- and down-regulated genes was determined in GT, MH, and HB cells. The LD-MESC is at E9.0, and therefore the E9.5 MH population was of interest since it arises from the LD. The HM shared pathways with both the HB and HM, and was therefore removed from initial analysis. Three software analysis-based approaches were employed to analyze gene expression data, REACTOME, DAVID, and ENRICHR. This analysis was validated by examining regulated pathways across software platforms. For example, VEGFR signaling was listed as the highest ranked upregulated pathway in ENRICHR, but MAPK, one of its downstream targets, was one of the highest ranked pathways with DAVID. Our differentially expressed gene list were first input into REACTOME, and obtained pie charts for upregulated and downregulated gene pathways in the GT, MH, and HB clusters (FIG. 6N-O). Each cell type was compared to the other two, to find up-regulated and down-regulated genes (log2fc>0.5, FDR<0.05). Both the up-regulated and down-regulated pie charts were created and differences compared. The pic chart depicting GT upregulation (898 differentially expressed genes) was found to have a larger percentage of Disease, Metabolism of RNA, Developmental Biology, and Cellular Response genes compared to the upregulated MH and HB pie charts (FIG. 6N). Immune System genes were found to make up a larger proportion in the downregulated in GT pic chart (1104 differentially genes) (FIG. 6O). In addition to the analysis of signaling in the GT. MH, and HB clusters, upregulated and downregulated pathways were examined after comparing the GT. MH, HB, and HM populations (FIGS. 6P-U). The pie chart depicting GT upregulation (898 differentially expressed genes) had a larger percentage of Disease, Metabolism of RNA, Developmental Biology, and Cellular Response genes compared to the upregulated MH and HB pie charts. Immune System genes were found to make up a larger proportion in the downregulated in GT pie chart (1104 differentially genes) (FIG. 6O). These data suggest that similar to MH populations, the HM populations also exhibit upregulated migration genes, and signaling pathways, but had downregulated liver differentiation genes, in contrast to the HB population. (Sup. FIGS. 1P-U). These data also suggest complexity in terms of metabolic pathways that are downregulated in GT and upregulated in HB (FIGS. 6Q-R). The heatmap analysis demonstrated clear, global, differences in the MH population, as demonstrated by this analysis of liver differentiation (FIG. 6V). Importantly, it was observed that a high correlation the coordinated changes in gene expression, and as an example, show the high correlation between Hippo pathway and Branching Morphogenesis (FIG. 6W).

FIG. 66 shows bioinformatics analysis of mouse liver organogenesis. FIG. 66A Quality control parameters. The number of features indicates the number of genes detected in each cell. Gene count (nFeature_RNA. #of genes detected per cell). FIG. 6B The percent of mitochondria (% mitochondria equals percent of reads from mitochondrial genes) is the proportion of the reads that are mitochondria. Higher percentage of number of mitochondrial reads suggests dead cells. FIG. 6C TSNE (t-distributed stochastic neighbor embedding) plot made with the first 50 principal components which separated major cell populations. FIG. 6D Volcano plots for the differential expression analysis comparison between GT and MH and MH and HB. Green indicates the most significant (FDR<1×10⁻²⁰, log2fc>5) upregulated genes in MH. Red indicates the most significant (FDR<1×10⁻²⁰, log2fc>5) upregulated genes in GT. Blue indicates the most significant (FDR<1×10⁻²⁰, log2fc>5) upregulated genes in HB. FIG. 6E Original heatmap with top 15 most significant upregulated genes (FDR<1×10⁻²⁰, highest to lowest log2fc) for each cell type. FIG. 6F Same as FIG. 6E, except original heatmap labeled with markers used to refine each cluster. FIG. 6G Further quality control analysis steps. Raw scRNA-seq data was filtered and normalized, further divided into groups using a force-directed plot. The data was then regrouped based on highly variable markers that were strongly expressed in either GT (UBA52, RPL38) or MH (DHX9, HNRNPU). FIG. 6H Heatmap after re-clustering as in FIGS. 6E, FIG. 6F except after adjusting the clusters based upon the four markers shown in FIG. 6F. FIG. 6I A force-directed layout plot (n=2332 cells), with the original positional data and clusters FIG. 6J Same as FIG. 6I except new refined groups. FIG. 6K Markers used in the regrouping analysis are shown on a force-directed layout plot. UBA52 and RPL38 are used for GT, and DHX9 and HNRNPU are used for MH. FIG. 6L Force-directed layout plot analysis of liver differentiation markers EpCAM and Dlk1 (epithelial to hepatic transition), and liver differentiation genes (FOXA1, FOXA2, FOXA3, AFP, HHEX, PROX1, HNF4A, ALB, CEBPA, TBX3). FIG. 6M A force-directed plot, labeling each cell by the cell phase. G1 indicates growth, G2M indicates either mitosis or the second growth phase, S represents DNA synthesis FIG. 6N Pie chart created with REACTOME to compare the proportions of gene categories for upregulated differentially expressed gene lists (log2fc>0.5, FDR<0.05) for GT compared to the other two cell types (MH and HB). Listed below the chart is total number of differentially expressed genes for each comparison and the number of gene occurrences. FIG. 6O Same as FIG. 6N except for downregulated genes. FIG. 6P Significantly enriched pathways (Gene Ontology Biological Process, Kegg Pathways) found for the GT vs. MH and HB significantly upregulated (log2fc>0.5, FDR<0.05) gene list analyzed with DAVID. Data were filtered for a P<0.05, FDR<0.3 and grouped based on pathway type (Signaling Pathways, Cancer Pathways, Migrating Pathways, Metabolic Pathways). FIG. 6Q Same as FIG. 6P except for downregulated genes in GT. FIG. 6R Same as FIG. 6P except for upregulated genes in the HB population. FIG. 6S Same as FIG. 6P except for downregulated genes in the HB population. FIG. 6T Significantly enriched pathways (Gene Ontology Biological Process, Kegg Pathways) found for the HM vs. GT, MH and HB significantly upregulated (log2fc>0.5, FDR<0.05) gene list analyzed with DAVID. Data were filtered for a P<0.05, FDR<0.3 and grouped based on pathway type (Signaling Pathways, Cancer Pathways, Migrating Pathways, Metabolic Pathways). FIG. 6U Same as FIG. 6T except for downregulated pathways. FIG. 6V Heatmap containing selected liver differentiation markers. FIG. 6W A comparison of average relative expression values for all the genes within the hippo signaling (Kegg) pathway and the average relative expression values for all the genes within branching morphogenesis (GO BP). All genes found to be in both gene lists were removed from the analysis. Each dot represents a cell between E8.5 and E10.5 for GT, MH, HB cell types.

Transcriptome analysis with a second dataset for early murine liver organogenesis validates bioinformatics analysis. The findings of the MH population were validated by analyzing a second scRNA-seq study of liver development (FIG. 7A-H). The GT population exhibited the very similar increases in signaling and migration pathways as the MH population in (FIG. 7C-D), suggesting that the gestation time of samples may have differed slightly. Additionally, the GT population demonstrated similar changes at the MH population, in terms of epithelial to hepatic transition (FIG. 7B), global changes in heatmaps across the same pathways analyzed (FIG. 7E-G), and similar changes in FOXA expression as last time (FIG. 7H). Taken together, these data indicate that signaling, morphogenesis, migration, hypoxia, metabolism, and differentiation follow coordinated changes in the MH population compared to the GT, and HB populations.

FIG. 77 shows bioinformatics analysis of mouse liver organogenesis. FIG. 7A Quality control parameters. The number of features indicates the number of genes detected in each cell. Gene count (nFeature_RNA, #of genes detected per cell). The percent of mitochondria (% mitochondria equals percent of reads from mitochondrial genes) is the proportion of the reads that are mitochondria. Higher percentage of number of mitochondrial reads suggests dead cells. FIG. 7B A force-directed plot showing expression of EpCAM (left) and Dlk1 (right). Red indicates higher expression and blue indicates lower expression. Higher expression of EpCAM is common in more epithelial-like cells while Dlk1 expression is more common in hepatic-like cells. FIG. 7C Heatmap with top 15 most significant upregulated genes (FDR<1×10⁻²⁰, highest to lowest log2fc) for each cell type. No reclustering was performed. FIG. 7D Significantly enriched pathways (Gene Ontology Biological Process, Kegg Pathways) found for GT population compared to the GT, LP, and HB significantly upregulated (log2fc>0.5, FDR<0.05) gene list analyzed with DAVID. Data were filtered for a P<0.05, FDR<0.3 and grouped based on pathway type (Signaling Pathways, Cancer Pathways, Migrating Pathways, Metabolic Pathways). Note that the GT population in this dataset resembles the MH population in the Lotto et al. dataset. FIG. 7E Relative expression heatmap for all genes in the KEGG oxidative phosphorylation with the GT, LP, and LB dataset cells. FIG. 7F Relative expression heatmap for all genes in the KEGG hippo signaling with the GT, LP, and LB dataset cells. FIG. 7G Pathway heatmap analysis. Each pathway was given an average relative expression value based on the average expression for all the genes within each pathway and all the cells for each cell type. Green indicates relatively higher expression and red indicates lower expression. The two asterisks indicate a p-value<0.001. FIG. 7H Also contains average relative expression for individual FOXA factors. The two asterisks indicate a p-value<0.001.

Design of a novel and reproducible protocol for hPSC-HB induction with continuous mesenchymal signaling. To model the LD, hPSC-derived DE was first induced via a commercially available kit STEMdiff kit under a 4-day hypoxic culture protocol. The DE significantly expressed DE-specific genes and proteins, including SOX17, FOXA2, and low AFP (FIG. 8A-B) but to improve morphology under hypoxic conditions, a modified protocol was used (FIG. 8C-E).

Immunostaining was performed on day 14 hPSC-HBs. To do this control experiments were performed for all key markers. Negative controls of liver antibodies were tested on MRC5 fibroblast (negative control) cells (FIG. 8F). Positive controls were tested on MRC5 fibroblasts (FIG. 8G). Negative and positive control immunostaining was performed on HUVEC cells (FIG. 8H). Next, both negative and positive control immunostaining was performed on HepG2 cells (FIG. 8I-J). To demonstrate that hPSC-HB cells treated with H+M can be cultured long term, immunostaining was performed of day 24 cells expressed AFP, ALB, and TBX3, and minimal/negative levels of SMA (FIG. 8K).

FIG. 8 shows characterization of hPSC-HB culture. FIG. 8A Morphology and immunochemistry of endoderm and hepatic progenitor cells. Left panel-Phase contrast image of day 4 endoderm morphology. Bar=200 μm. Middle panels-Sox-17 and Foxa2 staining of endoderm progenitor cells on day 4. Observable protein expression detected. FIG. 8B Bar graph of gene expression kinetics (qRT-PCR) of endoderm induction from hPSC. N=3 for each group compared. All comparisons between day 0 and day 4. October 4, P=0.000067, FOXA2, P=0.037, SOX 17, P=0.00029. Plotted is mean±SD. Significance (*) defined as P≤0.05. FIG. 8C Phase contrast images of cells during endoderm induction with in house protocol under hypoxic conditions. Day 4 cell shown. FIG. 8D Immunofluorescent staining. Top row—day 4 cells stained for OCT4/DAPI (left) and FOXA2/DAPI (right), Bar=200 μm. Bottom—day 4 cells stained for SOX17/DAPI (left), and AFP/DAPI (right), Bar=200 μm. Observable protein expression detected. FIG. 8E Bar graph of gene expression kinetics (qRT-PCR) of endoderm transcription factors (TFs) during endoderm induction from human stem cells, on day 0 and day 4 of culture. Cells transfected on day 1 with siRNA. In control (scramble) and siFoxa1/2 conditions. N=3 for control and siFOXA1/2 for all conditions. OCT4, P=0.000063, SOX17, P=0.0015, FOXA2, P=0.012, GATA4, P=NS (P=0.011), GSC, P=NS. Plotted is mean±SD. Significance (*) defined as P≤0.05. FIG. 8F Immunostaining of MRC-5 fibroblast cells cultured in monolayer using same methods described above for negative control targets specifically: 2Ab°, Rabbit IgG, Mouse IgG2aK. AFP, ALB, FOXA2, and HNF4α. Immunostaining of MRC-5 fibroblast cells using

Secondary Antibody (goat anti-rabbit) alone. Images are taken at 10×. From left to right: left (DAPI), middle (2 Ab°), right (Merged images). No observable protein expression detected in cells. Scale bar=200 μm. Immunostaining of MRC-5 fibroblast cells using Rabbit IgG. Images are taken at 10×. From left to right: left (DAPI), middle (Rabbit IgG), right (Merged images). No observable protein expression detected in cells. Scale bar=200 μm. Immunostaining of MRC-5 fibroblast cells using Mouse IG2a Kappa. Images are taken at 10×. From left to right: left (DAPI), middle (Mouse IgG2aK), right (Merged images). No observable protein expression detected in cells. Scale bar=200 μm. Immunostaining of MRC-5 fibroblast cells for alpha-fetoprotein (AFP). Images are taken at 10×. From left to right: left (DAPI), middle (AFP), right (Merged images). No observable expression of protein detected in cells. Scale bar=200 μm. Immunostaining of MRC-5 fibroblast cells for albumin (ALB). Images are taken at 10×. From left to right: left (DAPI), middle (ALB), right (Merged images). No observable expression of protein detected in cells. Scale bar=200 μm. Immunostaining of MRC-5 fibroblast cells for Forkhead Box A2 (FOXA2). Images are taken at 10×. From left to right: left (DAPI), middle (FOXA2), right (Merged images). No observable expression of protein detected in cells. Scale bar=200 μm. Immunostaining of MRC-5 fibroblast cells for hepatocyte nuclear factor 4 alpha (HNF4A). Images are taken at 10×. From left to right: left (DAPI), middle (HNF4A), right (Merged images). No observable expression of protein detected in cells. Scale bar=200 μm. FIG. 8G Immunostaining of MRC-5 fibroblast cells cultured in monolayer using same methods described above for positive control targets specifically: β-actin, TBX3, and a-SMA. Immunostaining of MRC-5 fibroblast cells for β-actin. Images are taken at 5×. From left to right: left (DAPI), middle (β-Actin), right (merged images). Observable expression of protein detected in cells. Scale bar=500 μm. Immunostaining of MRC-5 fibroblast cells for T-box transcription factor 3 (TBX3). Images are taken at 10×. From left to right: left (DAPI), middle (TBX3), right (Merged images). Observable expression of protein detected in cells. Scale bar=200 μm. Immunostaining of MRC-5 fibroblasts cells for a-SMA. Images are taken at 10×. From left to right: left (DAPI), middle (α-SMA), right (Merged images). Observable expression of protein detected in cells. FIG. 8H Immunostaining of HUVEC cells cultured in monolayer using same methods described above for negative and positive controls specifically: 2Ab° and CD31. Negative immunostaining of Human umbilical vein endothelial cells (HUVEC) for platelet endothelial cell adhesion molecule (CD31) using secondary antibody only. Images are taken at 10×. From left to right: left (DAPI), middle (2 Ab° only), right (Merged images). Positive immunostaining of HUVEC cells for CD31. From left to right: left (DAPI), middle (CD31), right (Merged images). Observable expression of protein detected in cells. FIG. 8I Immunostaining of HepG2 hepatoblastoma cells cultured in monolayer using same methods described above for negative control targets specifically: 2Ab only. Rabbit IgG, OCT3/4, α-SMA, and CD31. Immunostaining of HepG2 hepatic carcinoma epithelial cells using secondary antibody (goat anti-rabbit) alone. Images are taken at 10×. From left to right: left (DAPI), middle (2 Ab°), right (Merged images). No observable expression of protein detected. Scale bar=200 μm. Immunostaining of HepG2 hepatic carcinoma epithelial cells for Rabbit IgG. From left to right: left (DAPI), middle (Rabbit IgG), right (Merged images). No observable expression of protein detected. Scale bar=200 μm. Immunostaining of HepG2 hepatic carcinoma epithelial cells for octamer-binding transcription factor 3/4 (OCT3/4). Images are taken at 5×. From left to right: left (DAPI), middle (OCT3/4), right (Merged images). Low observable protein expression detected in cells. Scale bar=200 μm. Immunostaining of HepG2 hepatoblastoma cells for alpha smooth muscle actin (α-SMA). Images are taken at 5×. From left to right: left (DAPI), middle (α-SMA), right (Merged images). Low observable protein expression detected in cells. Immunostaining of HepG2 hepatic carcinoma cells for (CD31). Images are taken at 10×. From left to right: left (DAPI), middle (CD31), right (Merged images). No observable protein expression detected in cells. Scale bar=200 μm. FIG. 8J Immunostaining of HepG2 hepatoblastoma cells cultured in monolayer using same methods described above for positive control targets specifically: β-actin, AFP, ALB, FOXA1, FOXA2, FOXA3, HNF4α, TBX3. Immunostaining of HepG2 for Beta actin (β-Actin). Images are taken at 5×. From left to right: left (DAPI), middle (β-Actin), right (Merged images). Immunostaining of HepG2 cells for alpha-fetoprotein (AFP), left (DAPI), middle (AFP), right (Merged images). Images are taken at 5×. Observable protein expression detected in cells. Scale bar=500 μm. Immunostaining of HepG2 for albumin (Alb), left (DAPI), middle (Alb), right (Merged images). Images are taken at 5×. Observable protein expression detected in cells. Scale bar=500 μm. Immunostaining of HepG2 hepatic cells for Forkhead Box A1 (FOXA1). Images are taken at 10×. From left to right: left (DAPI), middle (FOXA1), right (Merged images). Observable protein expression detected in cells. Scale bar=200 μm. Immunostaining of HepG2 cells for Forkhead Box A2 (FOXA2). Images are taken at 10×. From left to right: left (DAPI), middle (FOXA2), right (Merged images). Immunostaining of HepG2 cells for Forkhead Box A3 (FOXA3). Images are taken at 10×. From left to right: left (DAPI), middle (FOXA3), right (Merged images). Observable protein expression detected in cells. Scale bar=200 μm. Immunostaining of HepG2 cells for hepatocyte nuclear factor 4 alpha (HNF4α). Images are taken at 10×. From left to right: left (DAPI), middle (HNF4α), right (Merged images). Observable protein expression detected in cells. Scale bar=200 μm. FIG. 8K Phase contrast morphology of replated D14 H+M derived hepatoblast cells for an additional ten days, to day 24. From left to right: Left (D18 H+M), middle (D21 H+M), right (D24 H+M). Immunostaining of iPSC D24 H+M derived hepatoblast (HB) cells cultured in monolayer using same methods described above for positive and negative targets: 2Ab° (neg), AFP (pos), ALB (pos), TBX3 (pos) and α-SMA (neg). Immunostaining of D14 H+M cells for 2Ab only. Images are taken at 10×. From left to right: Left (DAPI), middle (2Ab only), right (Merged images). No observable protein detected. Scale bar=200 μm. Immunostaining of D14 H+M cells for AFP. Images are taken at 10×. From left to right: Left (DAPI), middle (AFP), right (Merged images). Observable protein detected in cells. Scale bar=200 μm. Immunostaining of D14 H+M cells for Albumin. Images are taken at 10×. From left to right: Left (DAPI), middle (Alb), right (Merged images). Observable protein detected in cells. Scale bar=200 μm. Immunostaining of D14 H+M cells for TBX3. Images are taken at 10×. From left to right: Left (DAPI), middle (TBX3), right (Merged images). Observable protein detected in cells. Scale bar=200 μm. Immunostaining of D14 H+M cells for α-SMA. Images are taken at 10×. From left to right: Left (DAPI), middle (α-SMA), right (Merged images). No observable protein detected in cells. Scale bar=200 μm. FIG. 8

Forming compact 3D hepatic organoids via exposure to mesenchyme signals. Monolayer cultured cells were used to produce compact organoids. Day 6 GT cells were used with success in the vivo experiments, since the HE in LD arises from the GT. When day 6 hPSC-derived GT cells were harvested, and organoid protocol employed to day 9, the cells did not form compact organoids in control medium (FIG. 9A, 9B top, left), and instead resulted in cell-clusters. However, the addition of HUVECs in EGM-2 medium at a 1:1 ratio with GT, resulted in organoid compaction of GT cells on day 9 (FIG. 9B top, middle), and fluorescent labeling demonstrated that most of the HUVECs clustered to the center in the compact organoids (FIG. 9B). It was assessed whether compaction was due to the presence of HUVECs or the EGM-2 medium. HUVECs were removed and surprisingly, it was found that GT cells had compacted into spherical and homogeneous organoids by day 9 in EGM-2 alone, establishing that EGM-2 medium alone can drive organoid compaction in this model, perhaps due to mesoderm differentiation of progenitors within the organoid (FIG. 9C). These preliminary studies demonstrated that HUVECs can improve compaction/condensation. To further support evidence that organoid compaction occurred, and that other mechanisms are not responsible for the phenotype (e.g., differential proliferation) observed, Vybrant DiD were used dyes to label hepatic progenitor cells in suspension on day 14 (FIG. 9D). The data demonstrates that cells uniformly compacted, with minimal residual cells or cell clustering (FIG. 9E). To confirm the phenotype of these organoids, whole organoid immunostaining was performed for liver specific genes, and demonstrated evidence of both AFP expression (FIG. 9F). Cell viability was also assessed within organoids and it was found that minimal evidence of cell death occurred on day 17 (FIG. 9G). In summary, this example demonstrated a robust method for liver organoid compaction that employs day 14 early hPSC-HBs in H+M medium, and hypoxic conditions.

FIG. 9A Schematic of 3D hPSC-derived organoid formation in 384 well ultra-low attachment plates using gut tube (GT) endoderm cells harvested on day 6. FIG. 9B Phase contrast image of showing medium effects upon organoid compaction/condensation of hPSC-derived GT endodermal cells. Left—Day 7 GT cells in basal medium, Middle—Day 9 organoids with 1:1 mixture of GT and HUVEC cells in 50% basal/50% EGM-2 medium Right—same as Middle panel except fluorescent image. GT—green, HUVEC—red. FIG. 9C Phase contrast image of showing medium effects upon organoid compaction/condensation of hPSC-derived GT endodermal cells. Left—Day 9 GT grown only in 50% basal/50% EGM-2 medium. Right—same as Left panel except higher magnification. No HUVEC are added. FIG. 9D Phase contrast images of day 14 and day 17 H+M treated, dye-labeled cells during hPSC-HB organoid formation in 384-well ultra-low attachment plates. Day 14 cells at starting point. FIG. 9E Phase contrast images of day 14 and day 17 H+M treated, dye-labeled cells during hPSC-HB organoid formation in 384-well ultra-low attachment plates. Day 17 after compaction. Organoids uniformly condense to form compact organoids. FIG. 9F Phase contrast and immunofluorescence staining of AFP on day 17 hPSC-HB organoids. Whole organoids were fixed and immunostained, with DAPI counterstaining. Cells are AFP positive throughout. FIG. 9G Live/dead images analysis on day 15 of H+M treated hPSC-HB organoid. Data shows minimal cell death.

Day 18 LD-MESC organoids from extracellular matrix droplets express an immature hepatic signature in the absence of maturating factors. It was hypothesized that the hLD-MESC organoids can activate ALB (hepatic) expression during migration as occurs in the murine LD, in the absence of formal instructive factors. To determine whether hepatic differentiation was enhanced, gene expression was analyzed in the hLD-MESC platform treated in control compared to H+M medium on day 18. In this case control medium is placed day 15-day 18. When the control and the H+M condition were compared on day 18, the H+M condition (migration), demonstrated significantly increased ALB expression and decreased AFP and TTR expression (FIG. 10A). TFs associated with differentiation, like FOXA2 and HNF4A, demonstrated no significant changes (FIG. 10A). On the other hand, PROX1 was significantly upregulated in H+M medium, and TBX3 was significantly downregulated (FIG. 10A). In summary, the H+M condition compared to control resulted in significantly higher ALB, PROX1, and significantly lower TTR and TBX3 expression. To further characterize the hLD-MESC organoids, immunostaining was performed. High magnification immunostaining demonstrates that AFP-positive migrating cords (FIG. 10B). Further, high magnification views demonstrated cord-like migrating ALB-positive collective cell strands (FIG. 10C). In summary, the hLD-MESC organoids express an immature hepatic signature.

FIG. 10 shows gene and protein expression of control and H+M organoids cultured in MG droplets. FIG. 10A Bar graphs demonstrating effects of culture medium on gene expression in control and H+M conditions on day 18 with p-values listed in bar graphs. Gene expression analysis was performed by qRT-PCR. Sample size was control (n=3) and H+M (n=3). Genes analyzed were ALB, AFP, TTR, FOXA2, HNF4a, PROX1, TBX3. Plotted is mean+SD. FIG. 10B Immunocytochemistry of Control (top) and H+M treated (Middle and lower) day 18 whole organoids in MG droplet culture for AFP. Cells were counterstained with DAPI and FITC channel was used. High magnification images taken of migrating cells. FIG. 10C Same as FIG. 10B except ALB.

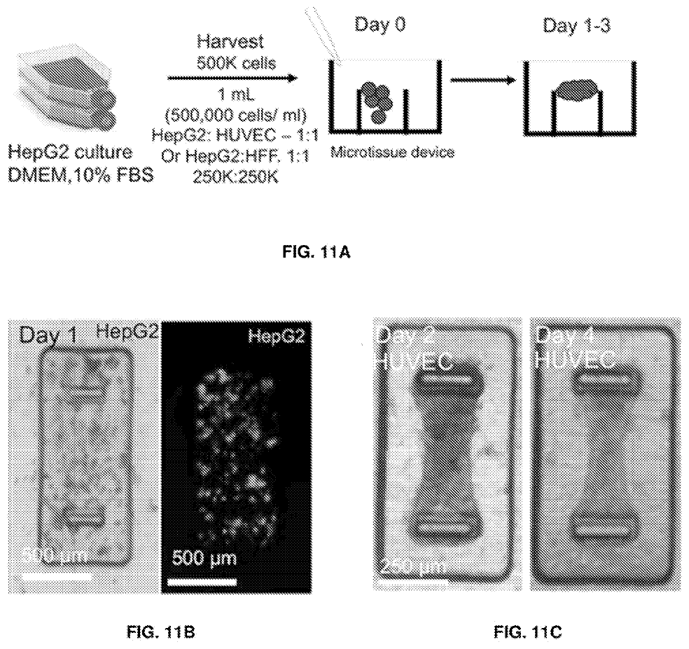

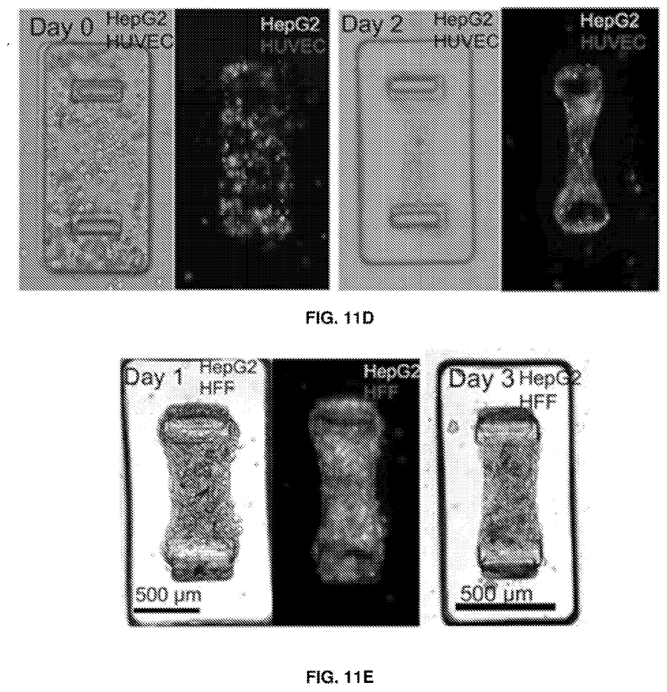

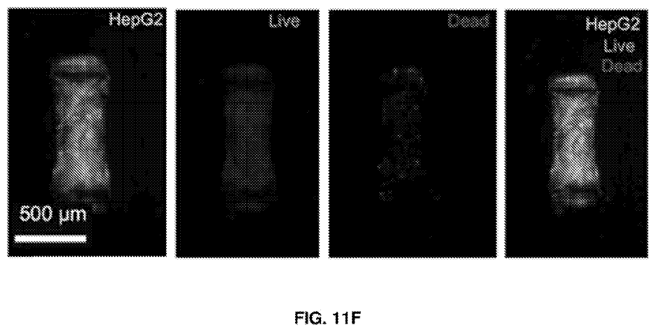

Early hPSC-HBs exhibit a functional mesenchymal phenotype in a functional assay with bioengineered tissue culture platform. Since it was observed that evidence of mesenchymal gene and protein expression in day 18 hLD-MESC model, it was hypothesized that cells had a functional, mesenchymal phenotype. To answer this, a functional assay was developed for mesenchymal phenotypes with liver cell populations. The hLD-MESC model was adapted for this system. The hLD-MES was integrated with a microfabricated device employing micropillars that provides a favorable environment for in vitro self-assembly of cell sheets from cultivated cells, together with both microscopic and biomechanical analysis. This cultivation system allows the calculation of contractile tension. A well-established, human hepatoma liver cell line was employed (HepG2) engineered to express eGFP and firefly luciferase in the microdevices, and when the cells were plated and submerged in a collagen gel, no self-assembly of cell sheets was observed (FIG. 11A-B). The HepG2 cells dispersed rather than forming a single uniform tissue (FIG. 11B). It was hypothesized that hepatic cells dispersed because of a lack of support, and that the addition of mesenchymal elements would improve cell sheet formation by providing mechanical support through cell-cell interactions and/or extracellular matrix secretion. First, HUVEC cells were plated alone in EGM-2 medium in the microfabricated pillar culture system, submerged in collagen gel, and observed cell sheets (FIG. 11C). Based on this data, cell sheet formation was improved by mixing HUVEC and HepG2 cells. By day 2, cell sheets had formed, suggesting HUVEC were responsible for the self-assembly of sheets through mechanical support (FIG. 11D). By employing fluorescent labeling and microscopy of both cell types, both cell types were identified, in a mixed cell and medium formulation, were present (FIG. 11D). To determine if this effect was endothelial-cell specific, HFF cells and a conventional serum-containing medium formulation that did not contain EGM-2 were employed. When submerged on collagen gel, it was found that tissue sheets had self-assembled, in this case a much thicker structure, and that both cell types were present (FIG. 11E). This data suggested that not only was the effect not dependent on HUVEC, but that in other cell types like HFF, EGM-2 was not required. To determine cell viability, live dead staining was performed, showing that greater than 95% of the cells were viable (FIG. 11F). These data indicate that MES-derived cells within the H+M treated population, like HFF and HUVEC, provide mechanical support through juxtacrine or paracrine cell-cell interactions, or extracellular matrix modification.

FIG. 11 shows hPSC-HB organoids form intact, contractile microtissues similar reminiscent of mesenchymal-epithelial tissues in functional microdevice. FIG. 11A Schematic of tissue self-assembly in microtissue array format. hPSC-HB, H+M treated day 14 monolayer cells are seeded onto PDMS array posts in collagen hydrogel for 3 days in H+M medium. FIG. 11B Phase contrast images of (left) and fluorescent (right) of HepG2-GFP cells (500 cells per microwell) on day 1 after seeding. No microtissue formed. FIG. 11C Phase contrast images of microtissue formed with HUVEC cells, on day 2 (left) and day 4 (right). FIG. 11D Phase contrast images (left) and fluorescent images (right) of HepG2-GFP and dye-labeled HUVEC cells seeded in microdevices at seeding of cells (day 0, left) and after two days (right). FIG. 11E Phase contrast images (left) and fluorescent images (right) of HepG2-GFP and dye-labeled HFF (red) cells seeded in microdevices on day 1. FIG. 11F Phase contrast images (left) and fluorescent images (right) of day 3 microtissues of HepG2-GFP and HFF. Live and dead staining performed. Live, dead, and merged image shown.

### EXAMPLE 4

This example demonstrates transcriptomic evidence demonstrates that a migration/growth stage arises from liver organoids and leads to boosted transcriptional maturity via growth differentiation switching, on par with the effect of transcription factor programming.

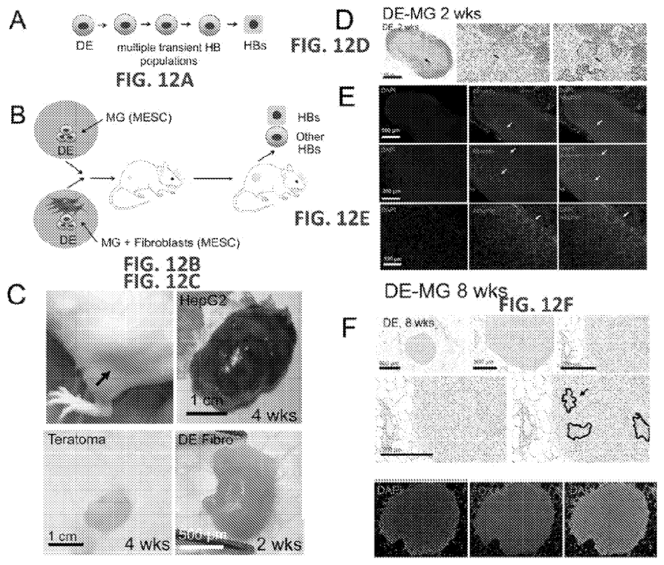

A human stem cell in vivo transplantation model of early liver growth to differentiation switching. We performed in vivo transplantation of early liver progenitors (DE/GT) to validate a model of growth to differentiation swith occurs in vivo (FIG. 12A). We developed a simple ectopic model in subcutaneous tissues within immunodeficient mice to evaluate whether DE/GT liver progenitors can lead to growth and differentiation (FIG. 12B). A subcutaneous site was chosen because it can support PSC-derived gut and liver tissue, is convenient to use, and is more likely hypoxic then commonly used transplant sites like kidney, capsule, brain, mesentery, which are highly vascularized and likely not hypoxic. Further, in these vascularized sites there are confounding factors which could contribute to growth and differentiation. Further, we reasoned that liver differentiation can occur via intrinsic, default mechanisms under hypoxia with no exogenous GFs as we observe in our spontaneous differentiation protocol, based upon the fact that early gut tube endoderm populations are already pre-primed for liver differentiation. We injected day 5 DE/GT cells with MG to model the MESC which resulted in a palpable mass after at least two weeks in all conditions tested (FIG. 12C). We validated this model with hPSC transplantation to form teratomas, which demonstrated positive and negative ALB staining (data not shown), and transplantation of HEPG2 cells, which also demonstrated ALB positive staining (data not shown) We tested our hypothesis that DE/GT transplantation can undergo a GDS and transform to hepatic cells. We transplanted day 5 DE/GT resulting in an oval-shaped mass with extensive homogeneous tissue growth after 2 weeks (FIG. 12D, top row). Higher magnification analysis demonstrated evidence of organization, with nests of cellular dense regions (FIG. 12D, top row). ALB immunohistochemistry for evidence of liver differentiation demonstrated a large part of the mass (>50%) was low positive for ALB, mainly focused on the center of the mass, which is noted in dual FITC-DAPI image (FIG. 12D, DAPI vs. Green). At the same time, there were clusters of cells, forming networks that expressed even higher ALB expression levels (FIG. 12D, arrows). This demonstrates in vivo liver differentiation occurs from DE/GT transplants without blood vessels and without instructive factors. We speculate that higher expressing cells may be undergoing the GDS, whereas the lower expressing cells only demonstrate spontaneous differentiation.

RNA-seq analysis of migration/growth demonstrates the unique MHB phenotype. Having observed migrating hepatoblasts (MHBs) in the mouse studies and evidence of a growth to differentiation switch (GDS) we wanted to engineer an hPSC protocol employs knowledge regarding GDS, as current protocols lack this step. Current protocols have 3 stages, including the DE induction, HB induction, and HB maturation. We focused on the HB induction stage, which we believe is lacking the GDS step, which could result in increased mugration and/or maturation. We built upon our recent published protocol which forms spontaneous HBs (SHBs) in monolayer without instructive factors. Here, we add mediators of liver regeneration, which are present in our MHB studies, in order to prime cells for growth/migration (FIG. 13A). The final medium was used continuously from day 5-18, and contains mediators of liver regeneration but no FBS, steroids, or traditional instructive or maturating factors (FIG. 13A), with continuous hypoxia. To design the medium, we used data from ENRICHR pathway analysis to determine that VEGF, EGF, FGF2, and IGF-1 signaling were upregulated in the mouse MHB population. Importantly, they also mediate liver regeneration and they are linked to liver differentiation and growth. Day 14 SHBs (spontaneous hepatoblasts (HBs) were subject to maintenance in monolayer culture until Day 18, or formation of organoids on day 15 (FIG. 13B). If organoids stayed in suspension, they formed LD-HB population (liver diverticulum hepatoblast) (FIG. 13B) and on day 15 liver organoids were transferred to matrigel droplets, resulting in either migration (MHB) or stopped migration in control medium (MHB-Control) (FIG. 13B). The four conditions were SHB, LD-HB, MHB, and MHB control.

Transcriptomic analysis of hPSC-HB populations demonstrates growth/migration and differentiation phenotypes. To determine which pathways dictate in vitro liver organoid growth/migration versus maturation, we performed RNA-seq analysis of all conditions. We conducted principle component (PC) analysis on our own data as well as published data, including hPSC, Gut Tube (GT), Fetal HEPs, Adult HEPs as controls (FIG. 13C). On Day 18, the data revealed distinct variations between different populations. LD-HB (subset 1 or LD-HB1), MHB, and SHB (sample 3) exhibited immaturity and more closely resembled GT. In contrast, SHB (samples 1 and 2), LD-HB2 (subset 2), and MHB-control populations displayed characteristics more akin to fetal HEPs (FIG. 13C).

To identify differences in populations, we examined liver master transcription factor (TF) expression, which together form the liver gene regulatory network (GRN), and liver differention genes, as a way to measure transcriptional differences. Interestingly, in the MHB and LD-HB1 populations, which were associated with migration and growth on the PCA plot. there was TBX3 upregulation, and global suppression of liver GRN TE, suggesting de-differentiation of HBs during migration (FIG. 13D). Lower levels of expression were observed for HNF4A, HNF1A, PROX1, and HNF1B (FIG. 13D). LD-HB1 exhibited nearly an exact phenotype Thus, the LD-HB1 demonstrated a unique liver GRN TF signature compared to the LD-HB2 group, with upregulation of TBX3 and global suppression of liver GRN TFs (FIG. 13D). On the other hand, LD-HB2 and MHB control demonstrated activation of liver gene expression, and expression of several liver GRN TF. LD-HB expressed FOXA2, FOXA3, HEX, PROX1, HNF6, CEBPA, HNF1A, whereas MHB control expressed alternate TFs (FIG. 13D). These data indicated similarities in the migrating populations (MHB and LD-HB1), but differences in the mature populations (MHB control and LD-HB2). We hypothesized that Neural TF and Mesoderm TF were upregulated in human MHB population. and our RNA-seq data confirmed this in MHB (FIG. 13E). We then confirmed the migration/growth (FIG. 13F) and differentiation/maturation phenotypes (FIG. 13G) using heatmap analysis across all populations. This data demonstrated strong global differences between these two states.

Analysis of global transcription demonstrates that MHB-Control and LD-HB2 population exhibit similar transcriptional maturity to directly programmed (TF programming) hepatocytes from fibroblasts. We aimed to determine differences in transcriptional maturity. We first examined liver GRN expression directly between the two mature populations, the LD-HB2 and the MHB-Control, demonstrating drastic differences (FIG. 14A), highlighting their differences. LD-HB2 expressed higher levels of FOXA2, FOXA3, GATA4, HNF1A, HNF1B, HNF6, HHEX, PROX1, and CEBPA. Alternatively, MHB control expressed higher levels of HNF4A, FOXA1, HNF1A, FOXA2 (low), CEBPA (low), and GATA4 (low). We wanted to further analyze differences, and thus we performed analysis of select signaling pathway differences between the two populations (FIG. 14B). We found that many pathways were upregulated in the LD-HB2 population but not the MHB-Control population, including VEGF. Epithelial to Mesenchymal transition, cyclic AMP, and PPARG signaling (FIG. 14B). On the other hand, DNA replication and oxidation reduction genes were up in the MHB-Control population (FIG. 14B). We hypothesized these cells are hepatic-like, and wished to develop a quantitative system to test maturity. We employed CellNet software (CellNet) to measure liver classification score, which can function as a measure of liver maturity. We input 23 published datasets, 3 controls, and the 5 experimental conditions. Based on this score, the order of liver differentiation, from least to most differentiated, was LD-HB1, MHB, SHB, MHB-Control, LD-HB2 (FIG. 14C). Importantly, MHB-Control and LD-HB2 were amongst the highest liver scores amongst the literature. Most studies added between 5-20 soluble factors and culture the cells 25-35 days or longer. Other studies employed Transcription factor programming of fibroblasts to hepatocytes. We wanted to compare our results to others in more detail. Direct comparison of our data to Du et al. and Xie et al, two transcription factor reprogramming studies, demonstrated that MHB-Control liver score was higher Du et al, and LD-HB2 liver score was higher than Xie at al (FIG. 14D). This suggests that our cells had the benefit of TF programming without any addition of either maturating factors or TF reprogramming.

To further examine the results in an unbiased fashion, we removed the weighted classification scores, and used an unweighted scoring system, in which the genes expressed were not weighted differently. We found that interestingly, MHB-Control was the highest FIG. 14E, normalized to human fetal HEPs). We further highlight this by plotting only our studies over number of genes tested (FIG. 14F). To further elucidate transcriptional maturity which highlights both LD-HB2 and MHB-Control, we examined CellNet classification scores when only using groups of 100 genes from liver classification gene list (FIG. 14G). Interestingly, the Tilson et al. study demonstrated the highest scores for the first 3 sets of 100 genes, but for the 4^{th} and 5^{th} groups, MHB-Control was the highest (FIG. 14G). For all 5 groups, MHB-Control was equal or higher than LD-HB2 (FIG. 14G). Since the CellNet Liver Classification Score used 646 genes, we performed heatmap analysis for these genes across all populations. MHB-Control expressed the highest levels of CellNet genes, and many genes were alternately expressed between MHB-Control and LD-HB2 (FIG. 14H). These studies demonstrate several important concepts. First, that transcriptional maturity and liver score may not be the same-the CellNet liver score weighs the data, transcriptional maturity accounts for what % of the 646 genes are actively being transcribed? In that case, MHB Control appears to have the highest transcriptional maturity. Further, our data indicates that our cells were as transcriptionally mature as cells that were programmed with 8 liver TFs/factors and 7 liver TFs/factors (Xie et al). This type of effect has never been reported before.

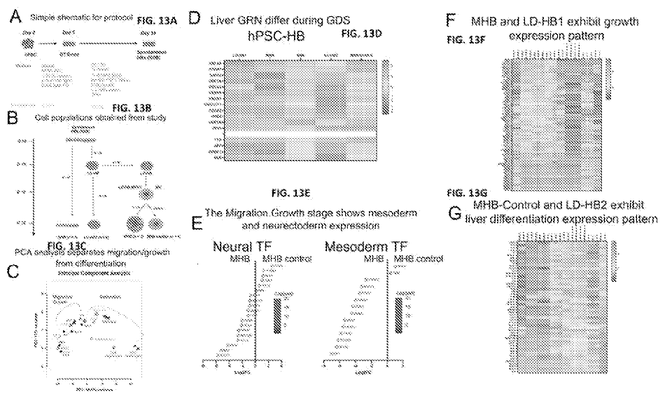

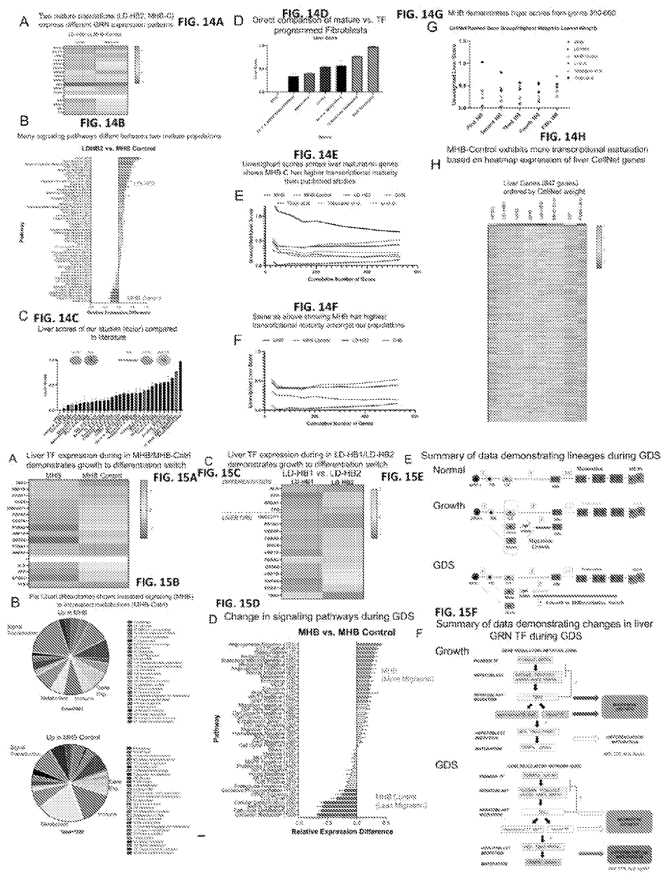

Evidence for migration/growth to differentiation switching as a mechanism of transcriptional maturation in HEPs. Epithelial organs, like the liver, lung, pancreas, and thyroid, exhibit transcriptional complexity, are therefore notoriously difficult to differentiate towards mature, functional cells. Further, these internal organs exhibit considerable growth and evidence of migration in vivo. These examples of migration followed by specification suggests a link between growth and differentiation that hasn't been formally explored in hPSC protocols. We first focused on MHB versus MHB-Control. MHB-Control is the same as the MHB population, but has been exposed to control medium for two days, and exhibits minimal migration compared to MHB. Two understand the effect of inhibiting migration, we first examined differences in liver GRN TF and liver differentiation genes between the MHB and MHB-Control conditions (FIG. 15A). Huge differences are noted which explain a potential growth to differentiation switching (GDS) mechanism. To further understand GDS in the MHB population, we performed REACTOME analysis followed by Pie chart analysis of upregulated genes between the two conditions (FIG. 15B). The data clearly shows that migration/growth is associated with increased signaling and gene transcription and decreased metabolism, whereas MHB control condition is associated with increased metabolism and downregulated signaling and transcription (FIG. 15B). We also analyzed gene expression differences between LD-HB1 and LD-HB2, which also demonstrate a differention switch (FIG. 15C). We examined pathway differences that may account for the GDS, and we observed that p53, Branching morphogenesis, and hedgehog were upregulated in the MHB population, whereas AMPK signaling, fatty acid metabolism, and Oxidatiaon reduction were all up in MHB-Control. (FIG. 15D). We provide two schematics, one with lineages (FIG. 15E), and one with TFs that explain our evidence of a GDS mechanism that functions as well as or better than TF programming of HEPs, for both MHB to MHB-Control, and LD-HB1 to LD-HB2.

Bioinformatics Analysis. Data Collection, Normalization and Filtering. Normalized data files were downloaded from the Single Cell Portal of the Broad Institute (singlecell.broadinstitute.org/single_cell). Annotation data for the different cell types and forced-directed layout data were also downloaded. The data was previously size-factor normalized using the computeSumFactors function in R with account to library size. Feature selection, dimensionality reduction, and doublet identification were additionally performed using the scran and scatter packages in R. and the data was log-transformed with the scater normalize function. Cells within the dataset were subsequently filtered based on key criteria for “unique features” (>1000 transcripts/cell) and “mitochondrial RNA content” (<20 per cell). These data were loaded into R with cell type data and further analyzed with the Seurat package. FindVariableFeature function was run with default dispersion function and mean functions was used.

Cell Cluster Regrouping. The Seurat function FindAllMarkers function was used to find globally enriched genes within each cell type. Default arguments were used except for a log2fc threshold of 0.5. Data was plotted ordered by log2fc with an adjusted-p-value cutoff of 1×10⁻²⁰. Two highly significant genes (log2fc>5, p-adj<1E-20) were selected based on differential gene expression to represent Gut Tube (GT) (UBA52, RPL38) or the MH (DHX99, HNRNPU) cell populations. The previous cell clusters were readjusted based upon additional clustering of cell that either expressed or did not express with these genes. Cells found with expression values over a standard deviation above expected compared to the cell type for both markers (UBA52 and RPL38 for GT, DHX9 and HNRNPU for MH) were regrouped to either GT or MH respectively.

For re-clustering, we used statistical analysis of 4 highly upregulated markers (two in the GT, two in the HB) to improve clustering (Sup. FIG. 1G) and performed a new heat map which had improved clustering (FIG. 1D, Sup. FIG. 1H, Table 1, Sup. Table 1). To determine marker expression across clusters, we employed the tools Harmony and Palantir and re-created force-directed plots (Sup. FIG. 1I-J). Harmony groups cells by cell type, and Palantir orders cells by pseudo-time and assigns probability for differentiation. The final force-directed plots demonstrate the DE to HB transition (Sup. FIG. 1E), the four re-grouping markers (Sup. FIG. 1K), liver differentiation genes (Sup. FIG. 1F) and major hepatic transcription factors (TFs) (Sup. FIG. 1L). The hepatic TFs HEX, TBX3, and PROX1 were nearly exclusively upregulated in the MH population, as expected (Sup. FIG. 1L). EHT was visualized by comparing EPCAM (Epithelial) to DLK1 (Hepatic) expression. The MH population had high DLK1 expression and low EPCAM expression (Sup. FIG. 1L). Since the MH population was tied to growth, we analyzed cell cycle with a cell phase plot, and found that MH cells, compared to the GT and HB populations, were more actively cycling in the G2-M (mitosis) and S phases (DNA synthesis) (Sup. FIG. 1M).

T-distributed stochastic neighbor embedding (TSNE). Principal components were found for all cell types (DE, GT, MH, HB, HM) using the normalized log count data for all the genes. The first 50 principal components were used to calculate the TSNE coordinates using the Seurat function, RunTSNE. The perplexity was set to 30. Data were graphed with a point size of 5 with Dimplot.

Pathway Heatmaps. Gene Set lists were downloaded from Mouse Genome Informatics (www.informatics.jax.org). The ScaleData function with a negative binomial model was used. The DoHeatmap function was then used with the scaled data and the gene set lists to create heatmaps for each gene set.

Average Heatmap. Average heatmap scores were determined by running the ScaleData function with a negative binomial model for all the genes within the Kegg or GEO biological process pathway. Genes within the dataset were not included in the further analysis. The scaled expression data was then averaged for all the genes in the pathway for each individual cell, and this averaged expression was averaged again for all the cells in each cell type. The colors were then set based on a Red-Green spectrum with an RGB color model. Listed in the scale in each figure, red indicates lower gene expression and green indicates higher gene expression.

Pathway validation. To validate pathways in the MH cluster, we examined the expression of five liver differentiation genes (ALB, AFP, HEX, PROX1, TBX3), and these correlated in all three databases (DAVID, REACTOME, ENRICHR).

Differential Expression Analysis-DAVID. The FindMarkers function in Seurat was used to find differentially expressed genes between different cell types. These gene lists were able to be further filtered for genes with a log2fc>0.5 and an adjusted-p-value less than 0.05. The Entrez gene symbols from these lists were loaded into the DAVID Bioinformatics Resources 6.8 Analysis Wizard. The Functional Annotation Tool was then used to find gene ontologies and pathways with significant enrichment. In DAVID, a Fisher's Exact test is used to measure gene-enrichment for a specific gene set. DAVID produces a p-value from this test, and this p-value is adjusted based on the Benjamini-Hochberg method. Kegg Pathways and Gene Ontology (Biological Processes) were used, and only gene sets with an adjusted p-value<0.3 were used in our analysis and plotted in bar graph format.

Differential Expression-ENRICHR. The same gene lists were used for the ENRICHR analyses as the DAVID analyses. The gene lists for the comparisons between the MH and the GT as well as the MH compared with the HB were used. Both downregulated and upregulated genes were tested separately. The Entrez gene symbols were loaded into ENRICHR. The ENRICHR gene list enrichment analysis tool was used to find significant transcription factors with the ENCODE and ChIP Enrichment Analysis (ChEA) Consensus TFs. Kegg 2021 Human, WikiPathway 2021, and GO Biological Process 2021 were the gene sets used for the pathway analysis. All data was combined into a single data table, with information about the source of the pathway and whether it was found for the upregulated or downregulated list. These data were then filtered to find gene sets with an adjusted p-value<0.3.

Comparison of DAVID and ENRICHR. DAVID and ENRICHR can receive human and mouse genes as input. Both contain gene-set libraries from several sources (Gene

Ontology, Kegg, Wiki Pathways, REACTOME, Biocarta, etc.). In addition to ontology and pathway libraries, ENRICHR additionally offers transcription, disease/drugs, cell type, and miscellaneous libraries to further analyze gene lists. Many of these pathways are exclusive to ENRICHR. For enrichment calculations, DAVID uses a modified Fisher Exact Test, called Expression Analysis Systematic Explorer (EASE), which is a more conservative test compared to the Fisher Exact Test. It calculates p-values after subtracting one gene from the List Hits (LH). These p-values were further adjusted with the linear step-up method of the Benjamini and Hochberg. ENRICHR uses a Fisher exact test, which is corrected with a z-score permutation background correction. This process uses many random input gene lists to compute a mean rank and standard deviation from the expected rank. From this calculation, it can calculate a z-score, which is further combined with the p-value to score the pathways.

Pie chart analysis. The FindMarkers function in Seurat was used to find differentially expressed genes between different cell types. These lists were found comparing GT vs. MH and HB, MH vs. GT and HB, and HB vs. GT and MH, for both upregulated and downregulated genes. These Entrez gene lists were loaded separately into the REACTOME 3.7 Analysis Tool. From the REACTOME analysis, the number of genes in each of the highest hierarchical gene type were used to create a pie chart.

Gene expression PCA plots. Gene expression plots were plotted using the FeaturePlot function with the normalized expression data. A blue-red expression was used with blue indicating higher relative expression and red indicating lower relative expression.

Pathway Ranking. We performed a total of 15 comparisons involving single (e.g., MH to HB), double (e.g., MH to GT and HB), triple (e.g., MH to DE, GT, and HB) to develop a list of ranked pathways, focusing on signaling and metabolism genes. We also performed single comparisons (e.g., MH to HB) only. For each comparison, we obtained an up-regulated and down-regulated list of pathways (KEGG and/or Biological Process). We then ranked the comparisons by the frequency in which each pathway appeared in each comparison performed and calculated an averaged FDR value for all the comparisons.

Heatmap Ranking. We then performed a scoring analysis by averaging the expression across all genes in a given pathway and performing a combined heat map analysis by comparing groups.

FIG. 12 shows in vivo transplantation protocol and in vitro protocol for modeling early LO. FIG. 12A. Illustration depicting stage specific induction of hepatic progenitor cells. From left to right; Definitive Endoderm (DE), Gut tube Endoderm (GT-Endo), Spontaneous Hepatoblast (SHB), Liver Diverticulum Hepatoblast (LD-HB), LD-HB mesodermal-derivatives cells (LD-HB-MESC), Migrating HB-MESC (MHB-MESC) and Hepatoblasts (HBs). FIG. 12B. Schematic of procedure for in vivo transplantation of human pluripotent stem cell-derived (hPSC)-derived cells in immunodeficient mice. Individually, 1×106 hPSC, hPSC-derived definitive endoderm (DE) cells were mixed with human foreskin fibroblasts (HFF), and with growth factor-free Matrigel (MG) and transplanted subcutaneously onto the hindlimb of NOD-SCID mice. After 4 weeks, subcutaneous tissues formed and were analyzed by qRT-PCR and histology. FIG. 12C. Gross images of excised hPSC-derived tissues. Top left—Teratoma from hPSC, 4 weeks after transplantation in athymic nude mice (outside). Top right—Human hepatoblastoma (HepG2) cells were transplanted in vivo and collected 4 weeks after transplantation. Bottom left—Teratoma tissue excised from 4-week transplant. Bottom right—hPSC-DE was combined with human foreskin fibroblasts (HFF) to create the DE:HFF condition in a 9:1 ratio prior to transplantation in athymic nude mice, tissue after 2-week transplantation. Scale bar in image. FIG. 12D. Histological analysis of hPSC-derived DE two weeks post transplant, demonstrating Growth to Differentiation switching in vivo of DE, 2 weeks after transplant. Left—4× images shown. Middle 10× image shown. Right—10× image again with illustration. Arrows denote regions of endodermal cells. Traces denote regions of tissue self-assembly. Scale bar in image. FIG. 12E. Immunofluorescence of hPSC-derived DE, 2 weeks after transplant. From left to right: Left (DAPI), middle (Albumin), Right (Merge). Top row—4× image, Middle row—10×, Bottom row—20×. Arrows denote regions of albumin positive cells. Scale bar in image. FIG. 12F. Top-Histological analysis of hPSC-derived DE: Fibroblast, 8 weeks after transplant. Left—20× images shown. Right—20× image shown. Arrows denote regions of endodermal cells. Traces denote regions of tissue self-assembly. Scale bar in image. Bottom-Immunofluorescence of hPSC-derived DE: Fibroblast, 8 weeks after transplant for ALB, FOXA2, CDX2, and FOXA2 (20×).

FIG. 13 shows RNA-seq analysis of migration/growth demonstrates a unique MHB phenotype. FIG. 13A. hPSC differentiation protocol for the five conditions. Details are in Figure. FIG. 13B. Cell populations, labels, and sample numbers generated from study. FIG. 13C. Principal component analysis of bulk RNA-seq data using significant liver and gut markers found by Mu et al. Our hPSC-derived cell populations (LD-HB1 (light blue), MHB (blue), SHB (red), LD-HB2 (dark blue), MHB Control (green)) were compared with controls for hPSC (orange), gut tube (yellow), 12 week fetal hepatocytes (purple), and adult hepatocytes (brown). The following datasets from the literature were additionally included, Li et al. (day 0-21, black), Velazquez et al. (day 0-17, pink), and Tilson et al. (day 21, dark green). FIG. 13D. Comparison of select liver GRN transcription factors, and liver differentiation genes, and their relative expression levels (green indicating high, red indicating low) for all cell populations used in study. FIG. 13E. Expression of Neural TFs and Mesoderm TFs in MHB (left) and MHB-Control (right). Data shows expression of several key factors including markers of EMT. FIG. 13F. Heat map demonstrating growth related gene list is upregulated in migrating (MHB and LD-HB1) populations. FIG. 13G. Same as F except for liver differentiation gene list. LD-HB2 and MHB-Control demonstrate high expression.

FIG. 14 shows evidence for boosted transcriptional maturation in mature MHB-Control and LD-HB2 populations. FIG. 14A. Heatmap of liver GRN TF and liver differentiation gene expression between mature MHB-Control and LD-HB2 populations. Clear differences are demonstrated FIG. 14B. Bar graph of select signaling pathways upregulated in LD-HB2 (above) versus MHB-Control. FIG. 14C. Bar graph of liver classification score (CellNet) for our five cell lines and 26 additional derived hepatocytes from the literature. Each condition is expressed as mean±SD. FIG. 14D. Same as above except comparing two TF reprogramming studies Du et al. and Xie at al. and the MHB-Control and LD-HB2. Each study used ˜6-8 TFs for reprogramming, while ours had a migration/growth stage prior to maturation. FIG. 14E. Liver classification score based on average unweighted gene score for CellNet liver markers, the gene score is measured based on transformed relative gene expression between 12 week fetal (1) and hPSC (0). Relative measure of transcriptional maturity. FIG. 14F. Same as E except showing LD-HB2, MHB-Control, SHB, and MHB populations in this study. FIG. 14G. Liver classification score similar to E) for binned groups of 100 genes sorted by CellNet weight FIG. 14H. Heatmap of 647 CellNet liver genes for populations in this study and controls.

FIG. 15 shows evidence for a migration/growth to differentiation switching (GDS) mechanism. FIG. 15A. Heatmap of liver GRN TF and liver differentiation gene expression between mature MHB-Control and MHB populations. Clear differences are demonstrated indication GDS. FIG. 15B. Pie charts created with REACTOME containing gene categories for both upregulated and downregulated DEG lists between MHB and MHB Control (log2fc>1.5, FDR<0.05). Total number of gene occurrences shown. FIG. 15C. Comparison of select important liver GRN TFs and liver maturation genes between LD-HB2 and LD-HB1. Evidence for GDS. FIG. 15D. Bar graph of select signaling pathways upregulated in MHB (above) versus MHB-Control, during GDS. FIG. 15E. Schematic explaining the GDS observed in this study with respect to cell lineage. GDS enables a huge boost in transcriptional maturation, by passing several steps in differentiation. FIG. 15F. Schematic explaining GDS with respect to liver GRN TF expression.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every clement specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding.” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
growing endodermic cells in an environment comprising a hepatic medium having less than 60 mmHg O₂ partial pressure to produce hepatoblasts, wherein the endodermic cells are exposed to the environment for at least 8 days.

2. The method of claim 1, wherein the endodermic cells are exposed to the environment for at least 10 days.

3. The method of any one of claim 1 or 2, wherein the endodermic cells are exposed to the environment for at least 14 days.

4. The method of any one of claims 1-3, wherein the hepatic medium comprises IGF, EGF, FGF2, VEGF, and heparin.

5. The method of any one of claims 1-4, wherein the hepatic medium comprises a serum replacement.

6. The method of any one of claims 1-5, wherein the hepatic medium is free of serum.

7. The method of any one of claims 1-6, wherein the hepatic medium is free of steroids.

8. The method of any one of claims 1-7, further comprising:
growing pluripotent stem cells in an initial medium to produce the endodermic cells.

9. The method of claim 8, wherein the pluripotent stem cells comprise human cells.

10. The method of any one of claim 8 or 9, comprising growing pluripotent stem cells in the initial medium for at least 4 days.

11. The method of any one of claims 8-10, wherein the initial medium induces the pluripotent stem cells to produce the endodermic cells.

12. The method of any one of claims 8-11, wherein the initial medium is free of steroids.

13. The method of any one of claims 8-12, comprising growing the pluripotent stem cells at less than 60 mmHg O₂ partial pressure.

14. The method of any one of claims 1-13, further comprising exposing the heptaoblasts to one or more of BMP4, higher FGF2, HGF, dexamethasone, oncostatin, or vitamin D.

15. The method of any one of claims 1-14, comprising growing the endodermic cells in a cell culture plate.

16. The method of any one of claims 1-15, comprising growing the endodermic cells in a bioreactor.

17. The method of any one of claims 1-16, wherein the heptaoblasts form a liver organoid.

18. The method of claim 17, further comprising inducing liver architecture in the liver organoid.

19. The method of any one of claim 17 or 18, further comprising inducing vascularization in the liver organoid.

20. The method of claim 19, comprising inducing vascularization by exposing the liver organoid to VEGF.

21. The method of any one of claims 17-20, further comprising implanting the liver organoid in a subject.

22. The method of claim 21, wherein the subject is human.

23. The method of any one of claim 21 or 22, comprising implanting the organoid in a hepatic region of the subject.

24. A method, comprising:
growing pluripotent stem cells in an environment comprising a hepatic medium having less than 60 mmHg O₂ partial pressure, wherein the cells are exposed to the environment for at least 4 days;
exposing the pluripotent stem cells to fibroblasts; and
exposing the pluripotent stem cells and fibroblasts to a basement membrane matrix.

25. The method of claim 24, wherein the fibroblasts are present at a ratio of between 2:1 and 6:1 of stem cells:fibroblasts.

26. The method of any one of claim 24 or 25, wherein the fibroblasts comprise foreskin fibroblasts.

27. The method of any one of claims 24-26, wherein the basement membrane matrix comprises Matrigel.

28. The method of any one of claims 24-27, wherein the basement membrane matrix comprises collagen.

29. The method of any one of claims 24-28, wherein the hepatic medium comprises IGF, EGF, FGF2, VEGF, and heparin.

30. The method of any one of claims 24-29, wherein the hepatic medium comprises a serum replacement.

31. The method of any one of claims 24-30, wherein the hepatic medium is free of serum.

32. The method of any one of claims 24-31, wherein the hepatic medium is free of steroids.

33. The method of any one of claims 24-32, comprising causing the pluripotent stem cells and the fibroblasts to form a liver organoid.

34. The method of claim 33, wherein the organoid exhibits liver architecture.

35. The method of any one of claim 33 or 34, further comprising implanting the organoid into a subject.

36. The method of claim 35, wherein the subject is human.

37. The method of any one of claim 35 or 36, comprising implanting the organoid in the hepatic region.

38. The method of any one of claims 24-37, wherein the pluripotent stem cells comprise human cells.

39. The method of any one of claims 24-38, comprising growing the pluripotent stem cells in a cell culture plate.

40. A method, comprising:
growing pluripotent stem cells and fibroblasts in an environment comprising a hepatic medium to form a structure; and
implanting the structure in the skin of a subject.

41. The method of claim 40, comprising growing the pluripotent stem cells and the fibroblasts in an environment having less than 60 mmHg O₂ partial pressure.

42. The method of claim 41, comprising growing the pluripotent stem cells and the fibroblasts in the environment having less than 60 mmHg O₂ partial pressure for at least 3 days.

43. The method of any one of claim 41 or 42, comprising growing the pluripotent stem cells and the fibroblasts in the environment having less than 60 mmHg O₂ partial pressure for at least 4 days.

44. The method of any one of claims 40-43, wherein the fibroblasts are present at a ratio of between 2:1 and 6:1 of stem cells:fibroblasts.

45. The method of any one of claims 40-44, wherein the fibroblasts comprise foreskin fibroblasts.

46. The method of any one of claims 40-45, wherein the hepatic medium comprises IGF, EGF, FGF2, VEGF, and heparin.

47. The method of any one of claims 40-46, wherein the hepatic medium comprises a serum replacement.

48. The method of any one of claims 40-47, wherein the hepatic medium is free of serum.

49. The method of any one of claims 40-48, wherein the hepatic medium is free of steroids.

50. The method of any one of claims 40-49, comprising growing the pluripotent stem cells and the fibroblasts in a basement membrane matrix.

51. The method of claim 50, wherein the basement membrane matrix comprises Matrigel.

52. The method of any one of claim 50 or 51, wherein the basement membrane matrix comprises collagen.

53. The method of any one of claims 40-52, wherein the subject is human.

54. The method of any one of claims 40-53, wherein the structure exhibits liver architecture.

55. The method of any one of claims 40-54, wherein the pluripotent stem cells comprise human cells.
